(19) United States
(12) Patent Application Publication
(10) Pub. No.: US 2018/0346534 A1
(43) Pub. Date: Dec. 6, 2018

(54) C-TERMINAL EPITOPES IN AMYLOID BETA AND CONFORMATIONALLY-SELECTIVE ANTIBODIES THERETO

(71) Applicant: The University of British Columbia, Vancouver (CA)

(72) Inventors: Neil R. Cashman, Vancouver (CA); Steven S. Plotkin, Vancouver (CA)

(21) Appl. No.: 15/774,759

(22) PCT Filed: Nov. 9, 2016
(86) PCT No.: PCT/CA2016/051301

§ 371 Date: May 9, 2018

Related U.S. Application Data
(60) Provisional application No. 62/253,044, filed on Nov. 9, 2015.
(60) Provisional application No. 62/352,346, filed on Jun. 20, 2016.
(60) Provisional application No. 62/365,634, filed on Jul. 22, 2016.
(60) Provisional application No. 62/393,615, filed on Sep. 12, 2016.

(51) Int. Cl. C07K 14/47; C07K 7/64; C07K 16/18; G01N 33/68; A61K 51/10; A61P 25/28

(52) U.S. Cl. CPC C07K 14/4711; C07K 7/64; C07K 16/18; A61K 39/00; A61K 51/1018; A61P 25/28; G01N 33/6896

(57) ABSTRACT

The disclosure pertains to C-terminal epitopes identified in A-beta, including conformational epitopes, antibodies thereto and methods of making and using immunogens and antibodies specific thereto.

### RELATED APPLICATIONS

This is a PCT application which claims the benefit of priority of U.S. Patent Application Ser. No. 62/253,044, filed Nov. 9, 2015; U.S. Patent Application Ser. No. 62/352,346, filed on Jun. 20, 2016; U.S. Patent Application Ser. No. 62/365,634, filed on Jul. 22, 2016; and U.S. Patent Application Ser. No. 62/393,615, filed on Sep. 12, 2016, each of which are incorporated herein by reference.

### FIELD

The present disclosure relates to C-terminal Amyloid beta (A-beta or Aβ) epitopes and antibodies thereto and more specifically to conformational A-beta epitopes that are predicted and shown to be selectively accessible in A-beta oligomers, and related antibody compositions and uses thereof.

### BACKGROUND

Amyloid-beta (A-beta), which exists as a 36-43 amino acid peptide, is a product released from amyloid precursor protein (APP) by the enzymes β and γ secretase. In AD patients, A-beta can be present in soluble monomers, insoluble fibrils and soluble oligomers. In monomer form, A-beta exists as a predominantly unstructured polypeptide chain. In fibril form, A-beta can aggregate into distinct morphologies, often referred to as strains. Several of these structures have been determined by solid-state NMR.

For, example, structures for several strains of fibrils are available in the Protein Data Bank (PDB), a crystallographic database of atomic resolution three dimensional structural data, including a 3-fold symmetric Aβ structure (PDB entry, 2M4J); a two-fold symmetric structure of Aβ-40 monomers (PDB entry 2LMN), and a single-chain, parallel in-register structure of Aβ-42 monomers (PDB entry 2MXU).

The structure of 2M4J is reported in Lu et al [8], and the structure of 2MXU is reported in Xiao et al [9]. The structure of 2LMN is reported in Petkova et al [10].

A-beta oligomers have been shown to kill cell lines and neurons in culture and block a critical synaptic activity that subserves memory, referred to as long term potentiation (LTP), in slice cultures and living animals.

The structure of the oligomer has not been determined to date. Moreover, NMR and other evidence indicates that the oligomer exists not in a single well-defined structure, but in a conformationally-plastic, malleable structural ensemble with limited regularity. Moreover, the concentration of toxic oligomer species is far below either that of the monomer or fibril (estimates vary but are on the order of 1000-fold below or more), making this target elusive.

Antibodies that bind A-beta have been described.

WO2010128139A1 titled BIOMARKERS AND METHODS FOR DIAGNOSING ALZHEIMER'S DISEASE AND/OR MILD COGNITIVE IMPAIRMENT discloses a diagnostic method for Alzheimer's disease through assessing levels of antibodies capable of binding pGlu A-Beta in a given subject's body fluid.

U.S. Pat. No. 9,273,126 B2 describes titled HUMANIZED ANTIBODIES AGAINST THE BETA-AMYLOID PEPTIDE an antibody to the A-beta sequence AIIGLMVGGVV (SEQ ID NO: 13) and a method for diagnosis.

WO2011033046A1 titled NOVEL ASSAY FOR THE DETECTION OF AMYLOID BETA PEPTIDES discloses a method for detection of A-beta (1-40).

EP1717250A1 titled MONOCLONAL ANTIBODY AND USE THEREOF discloses antibodies A-beta C-terminus sequences 35-40 MVGGVV (SEQ ID NO: 14) and 38-42 GVVIA (SEQ ID NO: 15) and uses thereof. The antibodies were made using peptides bound to thyroglobulin.

WO2014161875A1 titled METHOD FOR DETECTING Aβ-SPECIFIC ANTIBODIES IN A BIOLOGICAL SAMPLE discloses a method for detecting A-beta-specific antibodies using A-beta variants for the diagnosis of Alzheimer's disease.

WO2010015592A2 titled BIOASSAY FOR POLYQ PROTEIN and Weihofen et al. [12] describe a GGVV (SEQ ID NO: 1) C-terminal protein tag.

Paganetti et al. [11] describes the use of an A-beta1-40-specific monoclonal antibody against the free C-terminus peptide GGVV (SEQ ID NO: 1) of A-beta40 for the determination of A-beta 1-40 levels.

GGVV (SEQ ID NO: 1) has also been identified at the N-terminus of Parietaria officinalis major allergens through screening with a panel of monoclonal antibodies. [13].

Antibodies that preferentially or selectively bind A-beta oligomers over monomers or over fibrils or over both monomers and fibrils are desirable.

### SUMMARY

Described herein are epitopes and more particularly conformational epitopes, in A-beta comprising and/or consisting of residues GGVV (SEQ ID NO: 1) or related epitopes, and antibodies that specifically and/or selectively bind said epitopes. The epitopes may be selectively exposed in the oligomeric species of A-beta, in a conformation that distinguishes oligomeric species from that in the monomer and/or fibril.

An aspect includes a cyclic compound comprising: an A-beta peptide where the A-beta peptide comprises GVV and up to 6 A-beta contiguous residues, and a linker, wherein the linker is covalently coupled to the A-beta peptide N-terminus residue and the A-beta peptide C-terminus residue.

In an embodiment, the peptide is selected from GGVV (SEQ ID NO:1), GGVVI (SEQ ID NO:8), VGGVVI (SEQ ID NO:7), VGGVV (SEQ ID NO:6), and VGGV (SEQ ID NO:5).

In another embodiment, the cyclic compound is a cyclic peptide.

In another embodiment, the cyclic compound described herein, comprising i) a curvature of G and/or V in the cyclic compound that is at least 10%, at least 20%, or at least 30% different than the curvature compared to G and/or V in the context of a corresponding linear compound and/or the fibril; ii) at least one residue selected from G and V, wherein at least one dihedral angle of said residue is different by at least 30 degrees, at least 40 degrees, at least 50 degrees, at least 60 degrees, at least 70 degrees, at least 80 degrees, at least 90 degrees, at least 100 degrees, at least 110 degrees, at least 120 degrees, at least 130 degrees, at least 140 degrees or at least 150 degrees compared to the corresponding dihedral angle in the context of a corresponding linear compound and/or the fibril; and/or iii) has a conformation for V as measured by entropy that is at least 10%, at least 20%, at least 25%, at least 30%, at least 35%, at least 40% more constrained compared to a corresponding linear compound.

In another embodiment, the A-beta peptide is GGVVIA (SEQ ID NO:15).

In another embodiment, the cyclic compound further comprises a detectable label.

In another embodiment, the linker comprises or consists of 1-8 amino acids and/or equivalently functioning molecules optionally comprising one or more functionalizable moieties.

In another embodiment, the linker amino acids are selected from A and G, optionally wherein the functionalizable moiety is C.

In another embodiment, the linker comprises or consists of amino acids GCG. In another embodiment, the linker comprises a PEG molecule.

In another embodiment, the cyclic compound is selected from the following structures:

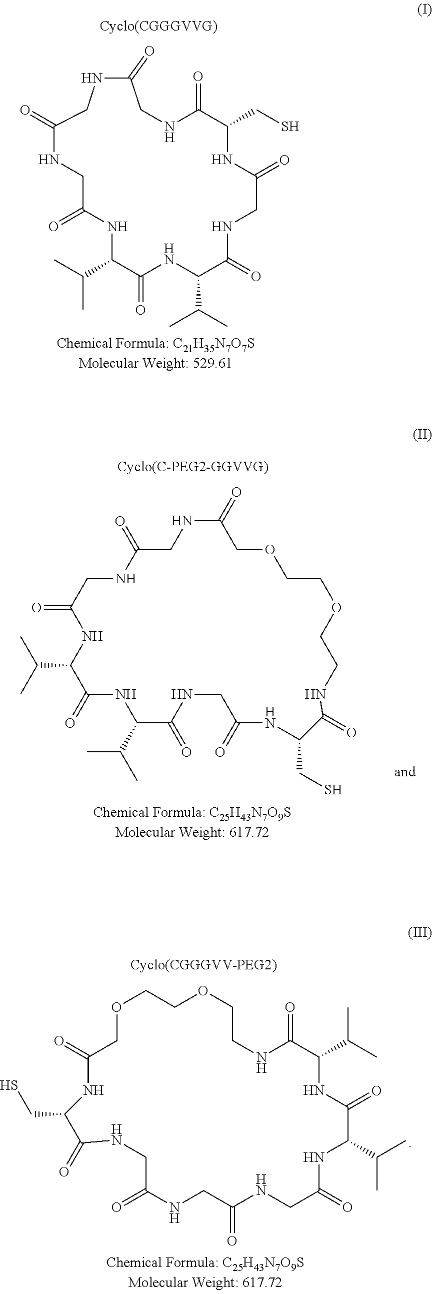 [SMILES: CC(C)[C@@H]1NC(=O)CNC(=O)CNC(=O)CNC(=O)[C@H](CS)NC(=O)CNC(=O)[C@H](C(C)C)NC1=O ; CC(C)[C@@H]1NC(=O)CNC(=O)CNC(=O)COCCOCCNC(=O)[C@H](CS)NC(=O)CNC(=O)[C@H](C(C)C)NC1=O ; CC(C)[C@@H]1NC(=O)CNC(=O)CNC(=O)CNC(=O)[C@H](CS)NC(=O)COCCOCCNC(=O)[C@H](C(C)C)NC1=O]

An aspect includes an immunogen comprising the cyclic compound described herein.

In an embodiment, the compound is coupled to a carrier protein or immunogenicity enhancing agent.

In an embodiment, the carrier protein is bovine serum albumin (BSA) or the immunogenicity-enhancing agent is Keyhole Limpet Haemocyanin (KLH).

An aspect includes a composition comprising the compound described herein or the immunogen described herein.

In an embodiment, the composition described herein, further comprises an adjuvant.

In an embodiment, the adjuvant is aluminum phosphate or aluminum hydroxide.

An aspect includes an isolated antibody that specifically binds to an A-beta peptide having a sequence of GGVV (SEQ ID NO:1) or a related epitope sequence, optionally as set forth in any one of SEQ ID NOS: 1-15.

In an embodiment, the antibody specifically and/or selectively binds an epitope in the A-beta peptide in the cyclic compound described herein compared to a corresponding linear compound.

In another embodiment, the epitope comprises or consists of at least two consecutive amino acid residues of GVV predominantly involved in binding to the antibody, wherein the at least two consecutive amino acids are GV embedded within GVV optionally GGVV (SEQ ID NO:1) or GGVVI (SEQ ID NO:8), wherein the at least two consecutive amino acids are GG embedded within GGV, optionally GGVV (SEQ ID NO:1) GGVVI (SEQ ID NO:8), or wherein the at least two consecutive amino acids are VV embedded within GVV, optionally GGVV (SEQ ID NO:1) or GGVVI (SEQ ID NO: 8).

In another embodiment, the A-beta peptide and/or epitope comprises or consists of GGVV (SEQ ID NO:1), GGVVI (SEQ ID NO:8), VGGVVI (SEQ ID NO:7), VGGVV (SEQ ID NO:6), and VGGV (SEQ ID NO:5).

In another embodiment, the antibody selectively binds to a cyclic compound comprising GGVV (SEQ ID NO:1) over a corresponding linear peptide.

In another embodiment, the antibody is at least 2 fold, at least 3 fold, at least 5 fold, at least 10 fold, at least 20 fold, at least 30 fold, at least 40 fold, at least 50 fold, at least 100 fold, at least 500 fold, at least 1000 fold more selective for the cyclic compound over the corresponding linear peptide.

In another embodiment, the antibody selectively binds A-beta oligomer over A-beta monomer and/or A-beta fibril.

In another embodiment, the antibody is at least 2 fold, 3 fold, at least 5 fold, at least 10 fold, at least 20 fold, at least 30 fold, at least 40 fold, at least 50 fold, at least 100 fold, at least 500 fold, at least 1000 fold more selective for A-beta oligomer over A-beta monomer and/or A-beta fibril.

In another embodiment, the antibody does not specifically and/or selectively bind a linear peptide comprising sequence GGVV (SEQ ID NO:1) or a related epitope, optionally wherein the sequence of the linear peptide is a linear version of a cyclic compound used to raise the antibody, optionally a linear peptide having a sequence as set forth in SEQ ID NO: 2, 3 or 4.

In another embodiment, the antibody lacks or has negligible binding to A-beta monomer and/or A-beta fibril plaques in situ.

In another embodiment, the antibody is a monoclonal antibody or a polyclonal antibody.

In another embodiment, the antibody is a humanized antibody.

In another embodiment, the antibody is an antibody binding fragment selected from Fab, Fab′, F(ab′)2, scFv, dsFv, ds-scFv, dimers, nanobodies, minibodies, diabodies, and multimers thereof.

In another embodiment, the antibody described herein, comprises a light chain variable region and a heavy chain variable region, optionally fused, the heavy chain variable region comprising complementarity determining regions CDR-H1, CDR-H2 and CDR-H3, the light chain variable region comprising complementarity determining region CDR-L1, CDR-L2 and CDR-L3 and with the amino acid sequences of said CDRs comprising the sequences:

| | | |
|---|---|---|
| CDR-H1 | GFTFSNYW | (SEQ ID NO: 17) |
| CDR-H2 | IRLKSYNYAT | (SEQ ID NO: 18) |
| CDR-H3 | LRWIDY | (SEQ ID NO: 19) |
| CDR-L1 | QDINSY | (SEQ ID NO: 20) |
| CDR-L2 | RAN | (SEQ ID NO: 21) |
| CDR-L3 | PQYDEFPYT | (SEQ ID NO: 22) |

In another embodiment, the antibody comprises a heavy chain variable region comprising: i) an amino acid sequence as set forth in SEQ ID NO: 24; ii) an amino acid sequence with at least 50%, at least 60%, at least 70%, at least 80% or at least 90% sequence identity to SEQ ID NO: 24, wherein the CDR sequences are as set forth in SEQ ID NO: 17, 18 and 19, or iii) a conservatively substituted amino acid sequence i).

In another embodiment, the antibody comprises a light chain variable region comprising i) an amino acid sequence as set forth in SEQ ID NO: 26, ii) an amino acid sequence with at least 50%, at least 60%, at least 70%, at least 80%, or at least 90% sequence identity to SEQ ID NO: 26, wherein the CDR sequences are as set forth in SEQ ID NO: 20, 21 and 22, or iii) a conservatively substituted amino acid sequence of i).

In another embodiment, the heavy chain variable region amino acid sequence is encoded by a nucleotide sequence as set forth in SEQ ID NO: 23 or a codon degenerate or optimized version thereof; and/or the antibody comprises a light chain variable region amino acid sequence encoded by a nucleotide sequence as set out in SEQ ID NO: 25 or a codon degenerate or optimized version thereof.

In another embodiment, the heavy chain variable region comprises or consists of an amino acid sequence as set forth in SEQ ID NO: 24 and/or the light chain variable region comprises or consists of an amino acid sequence as set forth in SEQ ID NO: 26.

In another embodiment, the antibody competes for binding to human A-beta with an antibody comprising the CDR sequences as recited in Table 13.

An aspect includes an immunoconjugate comprising the antibody described herein and a detectable label or cytotoxic agent.

In an embodiment, the detectable label comprises a positron emitting radionuclide, optionally for use in subject imaging such as PET imaging.

An aspect includes a composition comprising the antibody described herein or the immunoconjugate described herein, optionally with a diluent.

An aspect includes a nucleic acid molecule encoding a proteinaceous portion of the compound or immunogen described herein, the antibody described herein or a proteinaceous immunoconjugate described herein.

An aspect includes a vector comprising the nucleic acid described herein.

An aspect includes a cell expressing the antibody described herein and/or comprising the vector described herein.

An aspect includes a kit comprising the compound described herein, the immunogen described herein, the antibody described herein, the immunoconjugate described herein, the composition described herein, the nucleic acid molecule described herein, the vector described herein or the cell described herein.

An aspect includes a method of making the antibody described herein, comprising administering the compound or immunogen described herein or a composition comprising the compound or immunogen to a subject and isolating antibody and/or cells expressing antibody specific and/or selective for the compound or immunogen administered, and/or A-beta oligomers, optionally lacking or having negligible binding to a linear peptide comprising the A-beta peptide and/or lacking or having negligible plaque binding.

An aspect includes a method of determining if a biological sample contains A-beta, the method comprising:
a. contacting the sample with the antibody described herein or the immunoconjugate described herein under conditions permissive for forming an antibody: A-beta oligomer complex; and
b. detecting the presence of any complex.

In an embodiment, the biological sample contains A-beta oligomer the method comprising:
a. contacting the sample with the antibody described herein or the immunconjugate described herein that is specific and/or selective for A-beta oligomers under conditions permissive for forming an antibody: A-beta oligomer complex; and
b. detecting the presence of any complex;
wherein the presence of detectable complex is indicative that the sample may contain A-beta oligomer.

In another embodiment, the amount of complex is measured.

In another embodiment, the sample comprises brain tissue or an extract thereof, whole blood, plasma, serum and/or CSF.

In another embodiment, the sample is obtained from a human.

In another embodiment, the sample is compared to a control, optionally a previous sample.

In another embodiment, the level of A-beta is detected by SPR.

An aspect includes a method of measuring a level of A-beta in a subject, the method comprising administering to a subject at risk or suspected of having or having AD, an immunoconjugate described herein, wherein the antibody is conjugated to a detectable label; and detecting the label, optionally quantitatively detecting the label.

In an embodiment, the label is a positron emitting radionuclide.

An aspect includes a method of inducing an immune response in a subject, comprising administering to the subject a compound or combination of compounds described herein, optionally a cyclic compound comprising GGVV (SEQ ID NO:1) or a related epitope peptide sequence, an immunogen and/or composition comprising said compound or said immunogen; and optionally isolating cells and/or antibodies that specifically or selectively bind the A-beta peptide in the compound or immunogen administered.

An aspect includes a method of inhibiting A-beta oligomer propagation, the method comprising contacting a cell or tissue expressing A-beta with or administering to a subject in need thereof an effective amount of an A-beta oligomer specific or selective antibody or immunoconjugate described herein, to inhibit A-beta aggregation and/or oligomer propagation.

An aspect includes a method of treating AD and/or other A-beta amyloid related diseases, the method comprising administering to a subject in need thereof i) an effective amount of an antibody or immunoconjugate described herein, optionally an A-beta oligomer specific or selective antibody, or a pharmaceutical composition comprising said antibody; 2) administering an isolated cyclic compound comprising GGVV (SEQ ID NO:1) or a related epitope sequence or immunogen or pharmaceutical composition comprising said cyclic compound, or 3) a nucleic acid or vector comprising a nucleic acid encoding the antibody of 1 or the immunogen of 2, to a subject in need thereof.

In an embodiment, a biological sample from the subject to be treated is assessed for the presence or levels of A-beta using an antibody described herein.

In another embodiment, more than one antibody or immunogen is administered.

In another embodiment, the antibody, immunoconjugate, immunogen, composition or nucleic acid or vector is administered directly to the brain or other portion of the CNS.

In another embodiment, the composition is a pharmaceutical composition comprising the compound or immunogen in admixture with a pharmaceutically acceptable, diluent or carrier.

An aspect includes an isolated peptide comprising an A beta peptide consisting of the sequence of any one of the sequences set forth in SEQ ID NOS: 1-15.

In an embodiment, the peptide is a cyclic peptide comprising a linker wherein the linker is covalently coupled to the A-beta peptide N-terminus residue and/or the A-beta C-terminus residue.

In another embodiment, the isolated peptide described herein comprises a detectable label.

An aspect includes a nucleic acid sequence encoding the isolated peptide.

An aspect includes a hybridoma expressing the antibody.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIG. 1: Likelihood of exposure as a function of sequence, as determined by the Collective Coordinates method (Panel A) and the Promis Gō method (Panel B).

FIG. 2: Curvature as a function of residue index. Mean curvature in the equilibrium ensemble for the cyclic peptide CGGGVVG (SEQ ID NO: 2) is shown (Panel B), along with the curvature for the linear peptide (Panel A), and the curvature averaged over the various monomers in the fibril (Panel C). The convergence checks for the mean curvature values of all residues in each peptide is shown in Panels D-F.

FIG. 3: Dihedral angle distributions for the angle O-C-Cα-Hα1 (Panel A), O-C-Cα-Hα2 (Panel B), and O-C-Cα-N (Panel C), involving the side chain and backbone atoms of residue 38G, and for the angle and O-C-Cα-Cβ (Panel D), involving the side chain atoms of 40V are shown. Schematics of 38G and 40V are shown in the insets; the corresponding bond over which the dihedral angle is taken is rendered darker than the other bonds. The overlapping percentage values are provided in Table 2. The peaks of distributions are shown in Table 3.

FIG. 4: Entropy change of individual dihedral angles in the linear and cyclic peptides relative to the entropy in the fibril, plotted for each residue 37G (Panel A), 38G (Panel B), 39V (Panel C) and 40V (Panel D). Panel E: Side chain entropy of individual residues—the backbone Ramachandran entropy is not included. Panel F: Side chain plus backbone (total) conformational entropy of individual residues. The cyclic peptide is more rigid than the linear peptide for residues 39V and 40V. Low side chain conformational entropy in the cyclic peptide supports a well-defined conformational pose that could aid in conferring selectivity. Panel G plots the entropy loss of each residue relative to the linear peptide, showing explicitly that the entropy loss to be localized to the cyclic ensemble is significant. This indicates that it is rare for the linear peptide to adopt conformations consistent with the cyclic epitope. The probability to be in such a restricted set of conformations is approximately exp(−ΔS)≈0.001. The probability to be in the fibril conformation is enhanced by enthalpic compensation for the concomitant entropic loss.

FIG. 5: Equilibrium backbone Ramachandran angles for residue 38G, in cyclic (left panel) and linear (middle panel) forms of the peptide CGGGVVG (SEQ ID NO: 2), along with the backbone Ramachandran angles for the residue 38G in the context of the fibril 2MXU (right panel). The overlap probabilities between residue 38G in each linear, cyclic and fibril (2MXU) forms for Ramachandran angles are shown in Table 4. The peak angles of the corresponding distributions are shown in Table 5.

FIG. 6: Plots of the solvent accessible surface area (SASA), for the residues GGVV (SEQ ID NO: 1). The cyclic peptide is represented in dotted line. The linear peptide is represented in solid dark grey line. The fibril 2MXU is represented in solid light grey line.

FIG. 7: Panel A: Aligned centroid structures of residues 37G, 38G, 39V, and 40V in cyclic and linear peptides are shown in overlapping pictures from two different viewpoints. The cyclic peptide residues are shown in black, the linear peptide residues are shown in white. Panel B: Two views of the cyclic peptide structure CGGGVVG (SEQ ID NO: 2), and linear peptide structure CGGGVVG (SEQ ID NO: 2), both rendered in licorice representation so the orientations of the side chains can be seen. Panel C: Schematic representations of cyclic peptides containing the epitope residues GGVV (SEQ ID NO: 1), including the cyclic peptide CGGGVVG (SEQ ID NO: 2) with circular peptide bond, the cyclic peptide C-PEG2-GGVVG (SEQ ID NO: 3) with PEG2 linker between the G and C residues, and the cyclic peptide CGGGVV-PEG2 (SEQ ID NO: 4) with PEG2 linker between the C and V residues.

FIG. 8: The solvent-accessible surface area of the epitope GGVV (SEQ ID NO: 1) is shown for the linear and the cyclic peptides, and the C-terminus portion of Abeta40 polypeptide 2M4J.

FIG. 9: Clustering plots by root mean squared deviation (RMSD); axes correspond to the RMSD of GGVV (SEQ ID NO: 1) relative to GGVV (SEQ ID NO: 1) in the centroid structure of the cyclic peptide ensemble, the RMSD of GGVV (SEQ ID NO: 1) to GGVV (SEQ ID NO: 1) in the centroid structure of the linear peptide ensemble, and the RMSD of GGVV (SEQ ID NO: 1) to GGVV (SEQ ID NO: 1) in the centroid structure of the fibril ensemble of PDB ID 2MXU. Each point corresponds to a given conformation taken from either the cyclic peptide equilibrium ensemble, the linear peptide equilibrium ensemble, or the fibril equilibrium ensemble starting from PDB ID 2MXU. Three different viewpoints are presented in Panels A-C. The cyclic peptide ensemble, shown as dark gray circles, is conformationally distinct from either the linear or the fibril ensemble. Panels D-G show convergence checks of the overlap between the distributions of the cyclic, linear and fibril forms of the peptide. The numeric overlapping percentage is shown in Table 6. In particular, the cyclic peptide and the fibril peptide 2MXU have 0% overlap.

FIG. 10: Clustering plots by RMSD for other fibril strain conformations; axes correspond to the RMSD of GGVV (SEQ ID NO: 1) relative to GGVV (SEQ ID NO: 1) in the centroid structure of the cyclic peptide ensemble, the RMSD of GGVV (SEQ ID NO: 1) to GGVV (SEQ ID NO: 1) in the centroid structure of the linear peptide ensemble, and the RMSD of GGVV (SEQ ID NO: 1) to GGVV (SEQ ID NO: 1) in the centroid structure of the equilibrium ensembles for several fibril models of A-beta40. Each point corresponds to a given conformation taken from either the cyclic peptide, or various “strains” of fibril equilibrium ensembles, from PDB IDs 2M4J, 2LMN, and 2LMP.

FIG. 11: Primary Screening of clones from tissue culture supernatants using surface plasmon resonance (SPR) direct binding assay of tissue culture supernatants to cyclic peptide and linear peptide in Panel A, and A-beta oligomer and A-beta monomer in Panel B. Only IgG clones are shown.

FIG. 12: Plot comparing mAb binding to cyclic peptide in SPR direct binding assay versus ELISA. IgG, IgM, and IgA clones are shown.

FIG. 13: SPR direct binding assay of select clones to cyclic peptide, linear peptide, A-beta (Aβ) monomer, and A-beta oligomer (AβO).

FIG. 14: Immunohistochemical staining of plaque from cadaveric AD brain using 6E10 positive control antibody (A) and an antibody raised against cyclo(CGGGVVG) (SEQ ID NO: 2) (B).

FIG. 15: Secondary Screening of selected and purified antibodies using an SPR indirect (capture) binding assay. SPR binding response of pooled soluble brain extract (BH) from AD patients to captured antibody minus binding response of pooled brain extract from non-AD controls to captured antibody.

FIG. 16: Verification of Antibody binding to A-beta oligomers. SPR sensorgrams and binding response plots of varying concentrations of commercially-prepared stable A-beta oligomers binding to immobilized antibodies. Panel A shows results with the positive control mAb6E10, Panel B with the negative isotype control and Panel C with antibody raised against cyclo (CGGGVVG) (SEQ ID NO: 2). Panel D plots binding of selected antibody clones raised against cyclic peptide with A-beta oligomer at a concentration of 1 micromolar.

FIG. 17: A plot showing propagation of A-beta aggregation in vitro in the presence (stars) or absence (squares) of a representative antibody raised using a cyclic peptide comprising GGVV (SEQ ID NO: 1).

FIG. 18: A plot showing the viability of rat primary cortical neurons exposed to toxic A-beta oligomers (AβO) in the presence or absence of different molar ratios of a negative isotype control (A) or an antibody raised against cyclo (CGGGVVG) (SEQ ID NO: 2) (B). Controls include neurons cultured alone (CTRL), neurons incubated with antibody without oligomers and neurons cultured with the neuroprotective humanin peptide (HNG) with or without Aβ oligomers.

Table 1 shows the curvature value by residue of 37G, 38G, 39V, and 40V in linear, cyclic and fibril 2MXU forms.

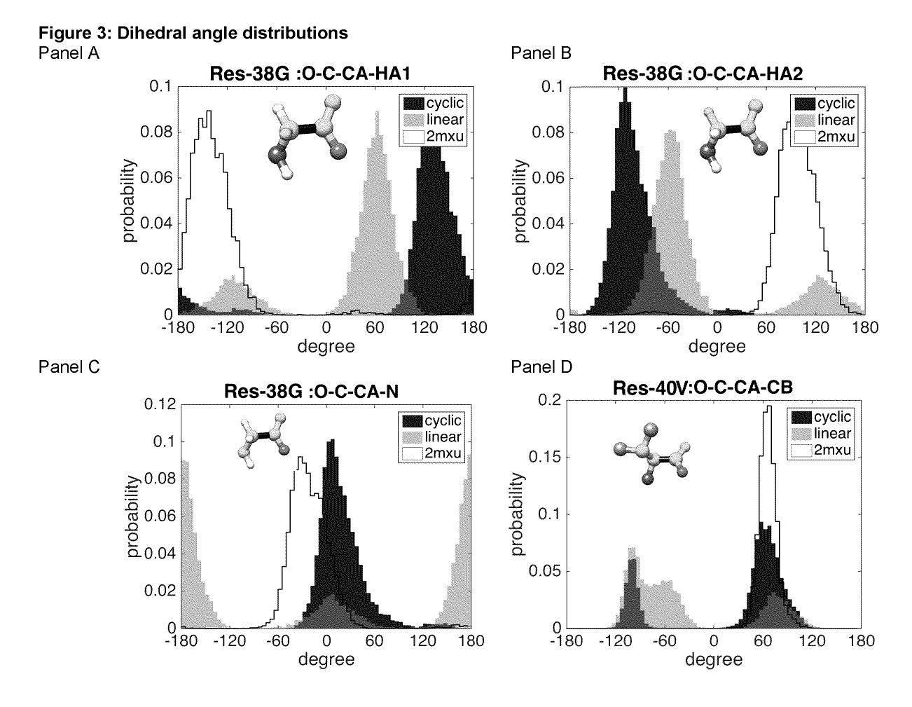

Table 2 shows the overlapping percentages of distribution in dihedral angles presented in FIG. 3.

Table 3 shows the peak values of the dihedral angle distribution for those dihedral angles whose distributions show significant differences between the cyclic peptide and other species. Column 1 is the specific dihedral considered, column 2 is the peak value of the dihedral distribution for that angle in the context of the linear peptide CGGGVVG (SEQ ID NO: 2), column 3 is the peak value of the dihedral distribution for that angle in the context of the cyclic peptide CGGGVVG (SEQ ID NO: 2), column 4 is the peak value of the dihedral distribution for the peptide GGVV (SEQ ID NO: 1) in the context of the fibril structure 2MXU, and column 5 is the difference of the peak values of the dihedral distributions for the linear and cyclic peptides. See FIG. 3.

Table 4 shows the overlap probabilities of Ramachandran angles of the residue 38G presented in FIG. 5.

Table 5 shows peak values of the Ramachandran backbone phi/psi angle distributions. The first column is the residue considered, which manifests two angles, phi and psi, indicated in parenthesis. The 2^{nd} column indicates the peak values of the Ramachandran phi/psi angles for residue 38G in the context of the linear peptide CGGGVVG (SEQ ID NO: 2), while the 3^{rd} column indicates the peak values of the Ramachandran phi/psi angles for residue 38G in the context of the cyclic peptide CGGGVVG (SEQ ID NO: 2), and the last column indicates the peak values of the Ramachandran phi/psi angles for 38G in the context of the fibril structure 2MXU. See FIG. 5.

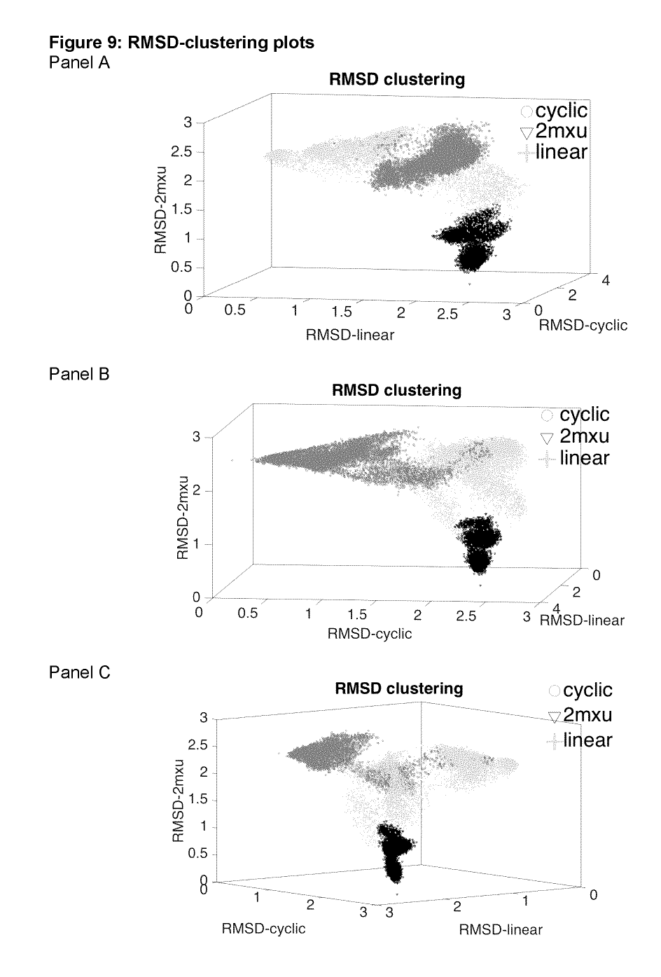

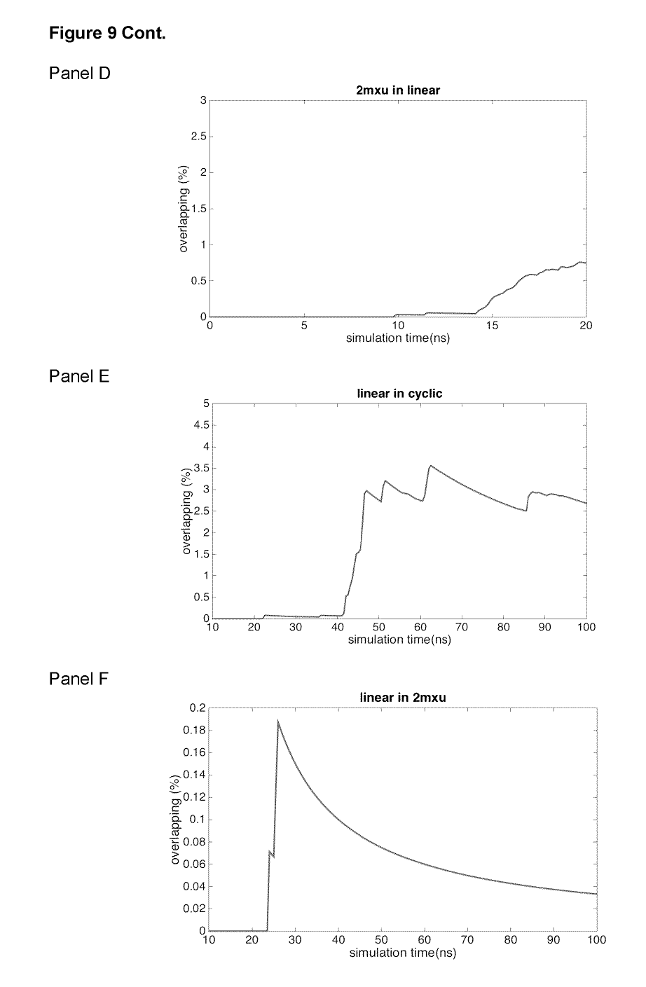

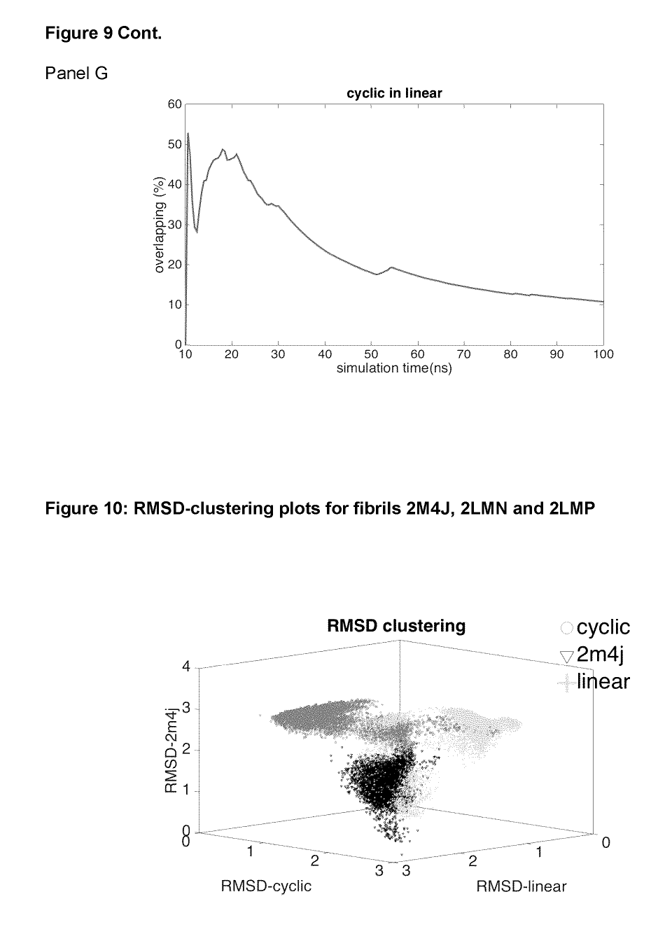

Table 6 shows the overlapping percentage of the RMSD clustering between the linear, cyclic and fibril (2MXU) forms of the peptide as presented in FIG. 9.

Table 7 gives the values of the backbone and sidechain dihedral angles for residues G37, G38, V39, and V40, in the centroid conformations of the cyclic, linear, and fibril ensembles. It also gives the difference in dihedral angles between the cyclic and linear centroid structures, and between the cyclic and fibril centroid structures.

Table 8 shows the binding properties of selected antibodies.

Table 9 shows the binding properties summary for selected antibodies.

Table 10 lists the oligomer binding-monomer binding for an antibody raised against cyclo(CGGGVVG) (SEQ ID NO: 2).

Table 11 lists properties of antibodies tested on formalin fixed tissues.

Table 12 is an exemplary toxicity assay

Table 13 lists CDR sequences.

Table 14 lists heavy chain and light chain variable sequences.

Table 15 is a table of A-beta “epitope” sequences and select A-beta sequences with linker.

Table 16 provides the amino acid sequence of A-beta 1-42.

### DETAILED DESCRIPTION OF THE DISCLOSURE

Provided herein are antibodies, immunotherapeutic compositions and methods which may target epitopes preferentially accessible in toxic oligomeric species of A-beta, including oligomeric species associated with Alzheimer's disease. A region in A-beta has been identified that may be specifically and/or selectively accessible to antibody binding in oligomeric species of A-beta.

As demonstrated herein, generation of oligomer-specific antibodies was accomplished through the identification of targets on A-beta peptide that are not present, or present to a lesser degree, on either the monomer and/or fibril. Oligomer-specific epitopes need not differ in primary sequence from the corresponding segment in the monomer or fibril, however they would be conformationally distinct in the context of the oligomer. That is, they would present a distinct conformation in terms of backbone and/or sidechain conformation in the oligomer that would not be present (or would be unfavourable) in the monomer and/or fibril.

Antibodies raised to linear peptide regions may not to be selective for oligomer, and thus may bind to monomer or A-beta plaques as well.

As described herein, to develop antibodies that may be selective for oligomeric forms of A-beta, the inventors sought to identify regions of A-beta sequence that are prone to disruption in the context of the fibril, and that may be exposed on the surface of the oligomer.

As described the Examples, the inventors have identified a region they have determined to be prone to disruption in the context of the fibril. The inventors designed cyclic compounds comprising the identified target region to satisfy criteria of an alternate conformation such as higher curvature, higher exposed surface area, alternative dihedral angle distributions, and/or did not readily align by root mean squared deviation (RMSD) to either the linear or fibril ensembles.

Antibodies could be raised using a cyclic peptide comprising the target region, that selectively bound the cyclic peptide compared to a linear peptide of the same sequence (e.g. corresponding linear sequence). Experimental results are described and identify epitope-specific and conformationally selective antibodies that bind synthetic oligomer selectively compared to synthetic monomers, bind CSF from AD patients preferentially over control CSF and/or bind soluble brain extract from AD patients preferentially over control soluble brain extract. Further staining of AD brain tissue identified antibodies that show no or negligible plaque binding and in vitro studies found that the antibodies inhibited Aβ oligomer propagation and aggregation.

### I. Definitions

As used herein, the term ‘A-beta’ may alternately be referred to as ‘amyloid beta’, ‘amyloid β’, Abeta, A-beta or ‘Aβ’. Amyloid beta is a peptide of 36-43 amino acids and as used herein includes all wild-type and mutant forms of all species, particularly human A-beta. A-beta40 refers to the 40 amino acid form; A-beta42 refers to the 42 amino acid form, etc. The amino acid sequence of human wildtype A-beta42 is shown in SEQ ID NO: 16.

As used herein, the term “A-beta monomer” herein refers to an individual subunit form of A-beta (e.g. 1-40, 1-41, 1-42, 1-43) peptide.

As used herein, the term “A-beta oligomer” herein refers to a plurality of any of the A-beta subunits wherein several (e.g. at least two) A-beta monomers are non-covalently aggregated in a conformationally-flexible, partially-ordered, three-dimensional globule of less than about 100, or more typically less than about 50 monomers. For example, an oligomer may contain 3 or 4 or 5 or more monomers. The term “A-beta oligomer” as used herein includes both synthetic A-beta oligomer and/or native A-beta oligomer. “Native A-beta oligomer” refers to A-beta oligomer formed in vivo, for example in the brain and CSF of a subject with AD.

As used herein, the term “A-beta fibril” refers to a molecular structure that comprises assemblies of non-covalently associated, individual A-beta peptides which show fibrillary structure under an electron microscope. The fibrillary structure is typically a “cross beta” structure; there is no theoretical upper limit on the size of multimers, and fibrils may comprise thousands or many thousands of monomers. Fibrils can aggregate by the thousands to form senile plaques, one of the primary pathological morphologies diagnostic of AD.

The term “GGVV” means the amino acid sequence glycine, glycine, valine, and valine as shown in SEQ ID NO: 1. Similarly GVV, GGV, VGGV (SEQ ID NO: 5), VGGVV (SEQ ID NO: 6), VGGVVI (SEQ ID NO: 7) and GGVVI (SEQ ID NO: 8), refer to the amino acid sequence identified by the 1-letter amino acid code. Depending on the context, the reference of the amino acid sequence can refer to a sequence in A-beta or an isolated peptide, such as the amino acid sequence of a cyclic compound.

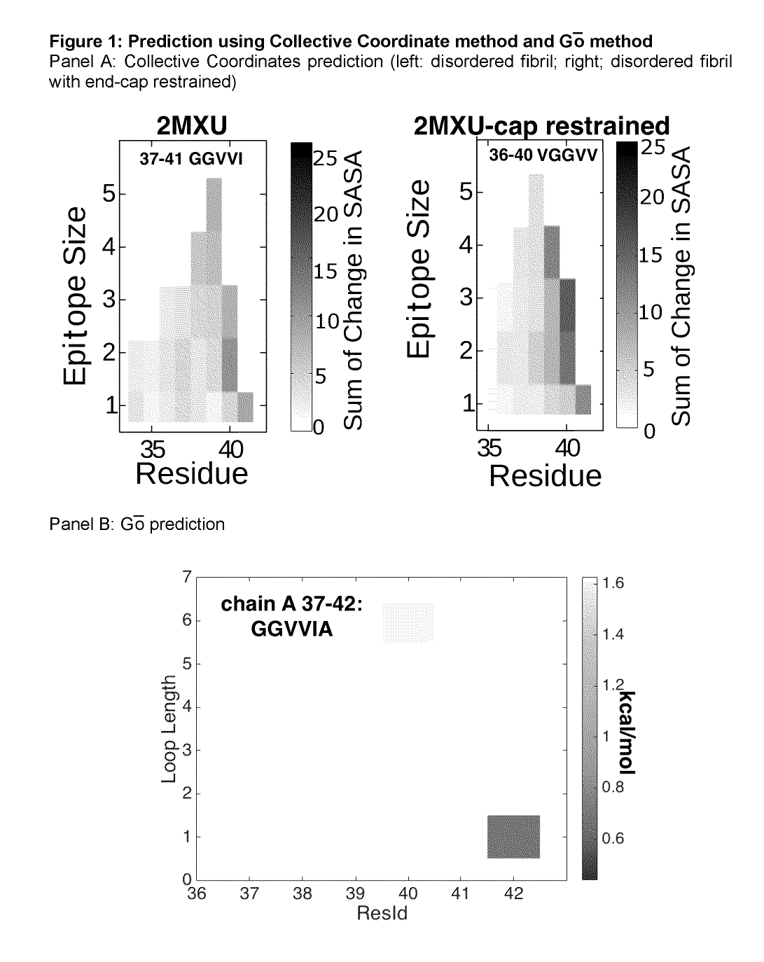

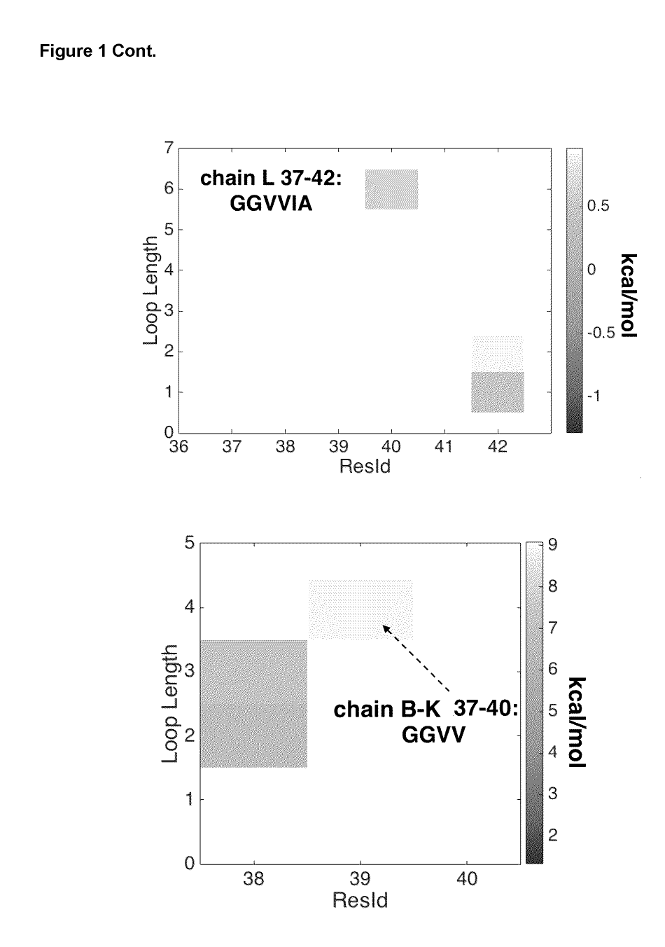

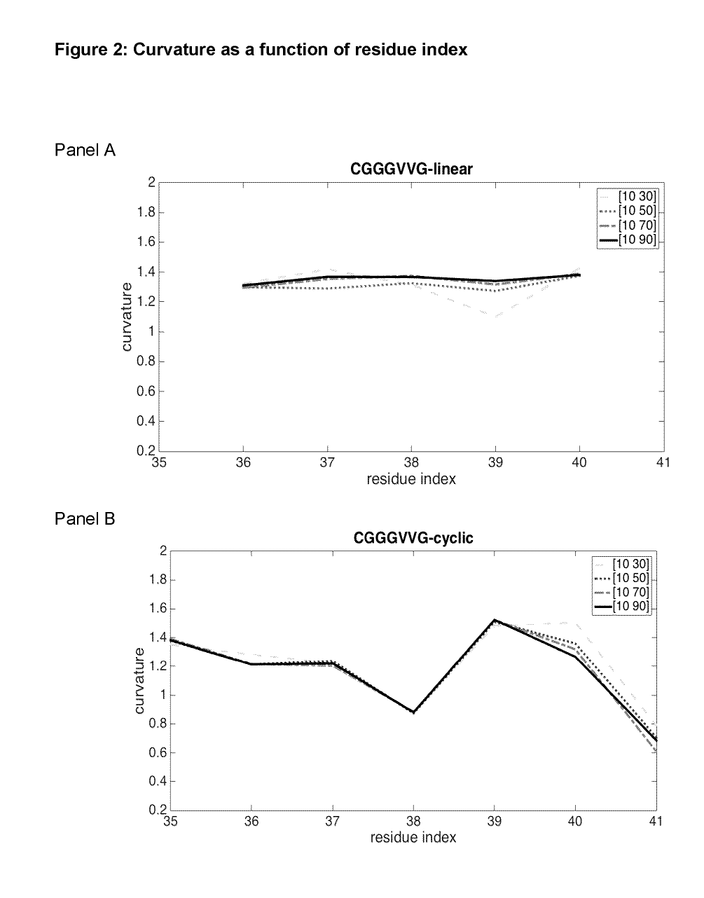

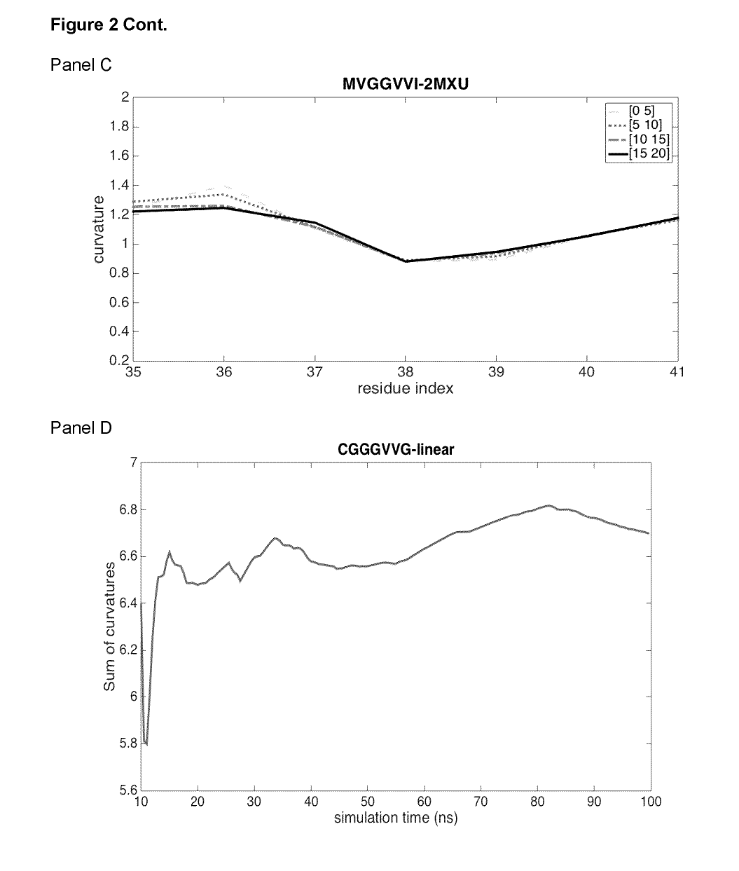

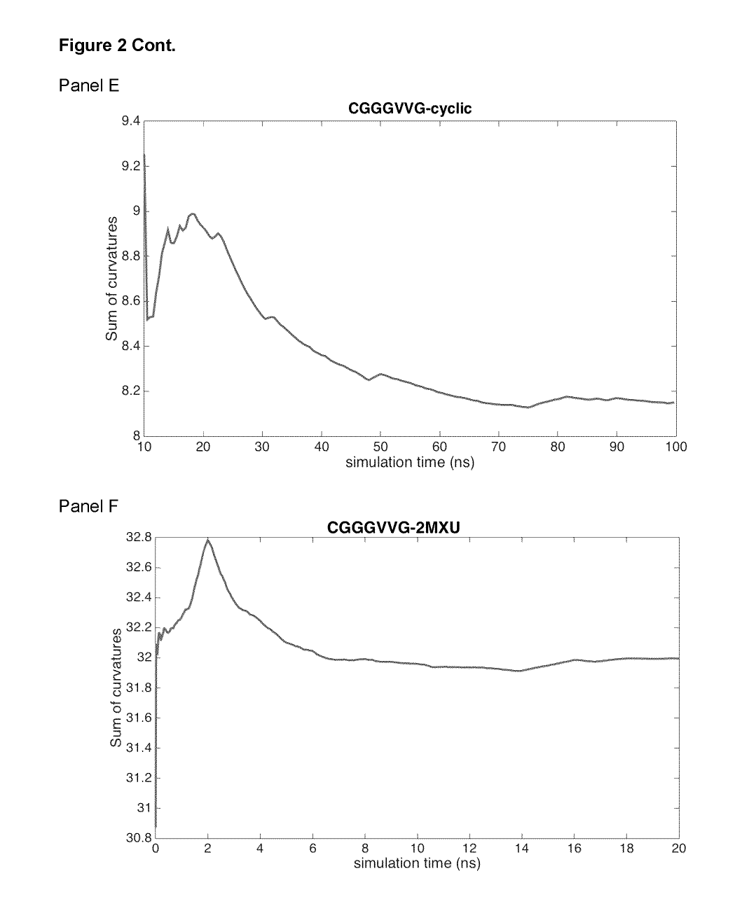

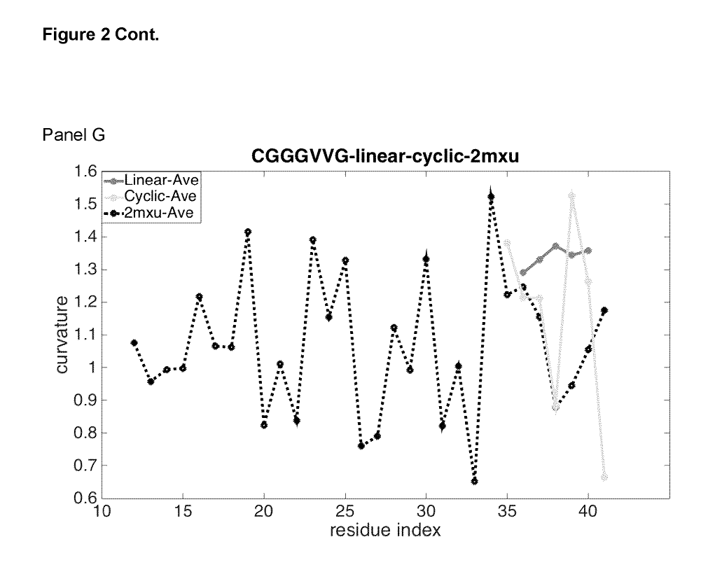

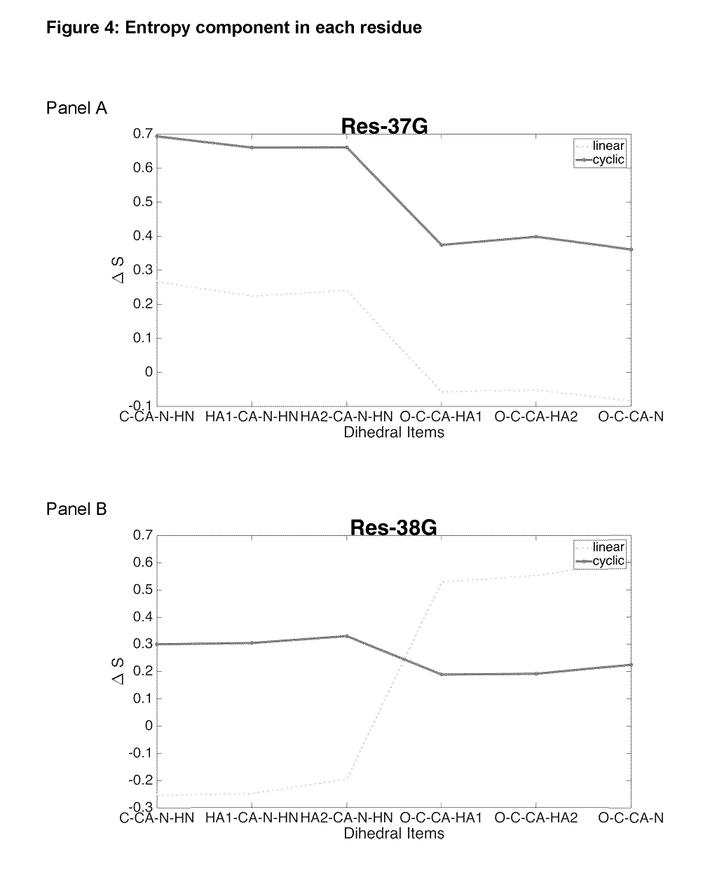

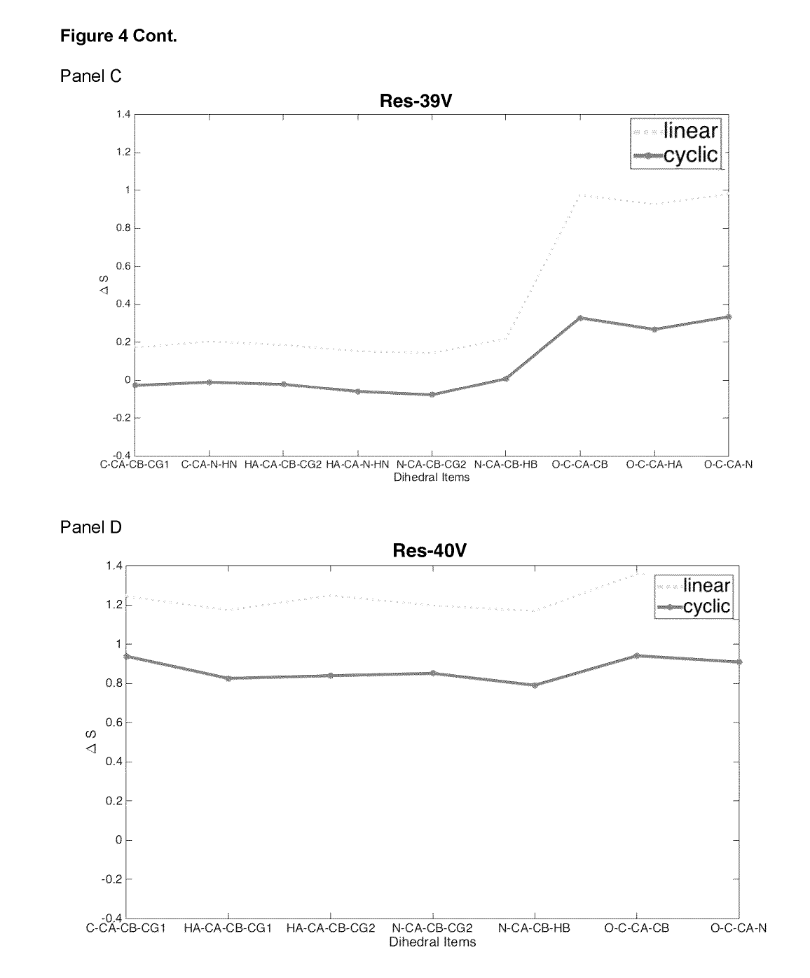

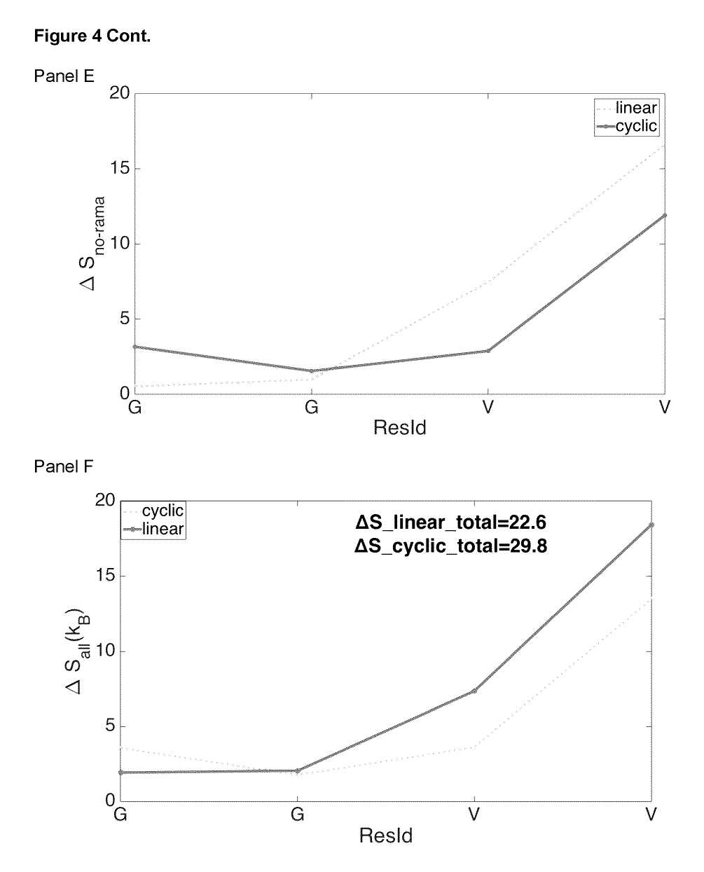

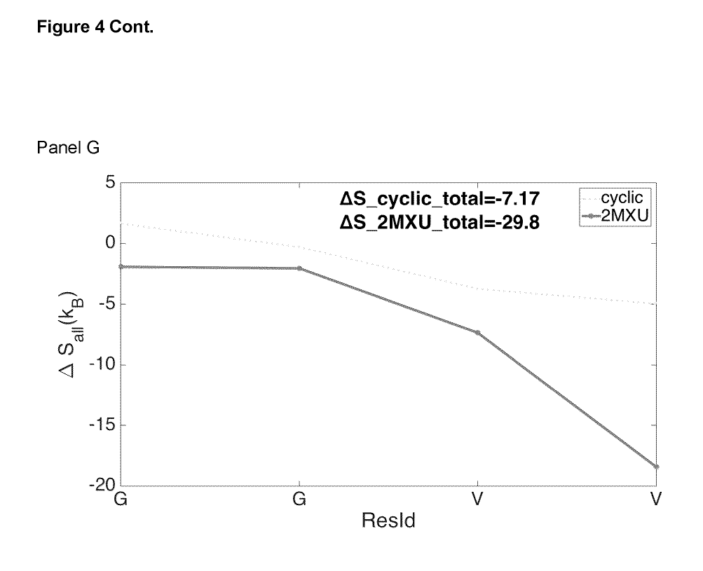

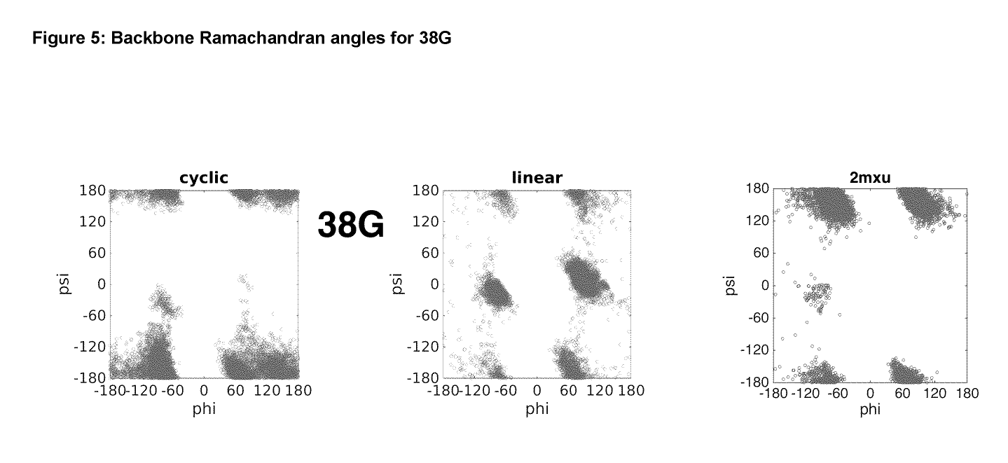

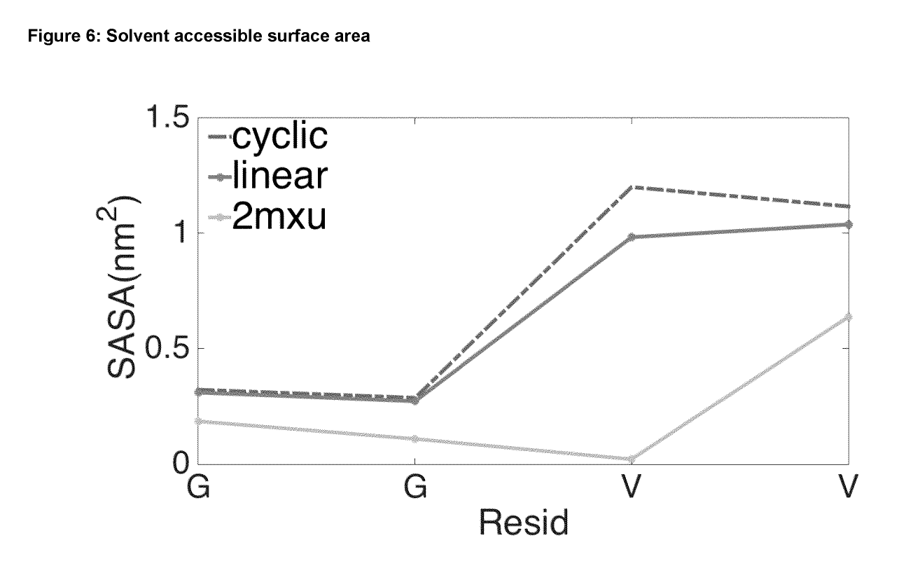

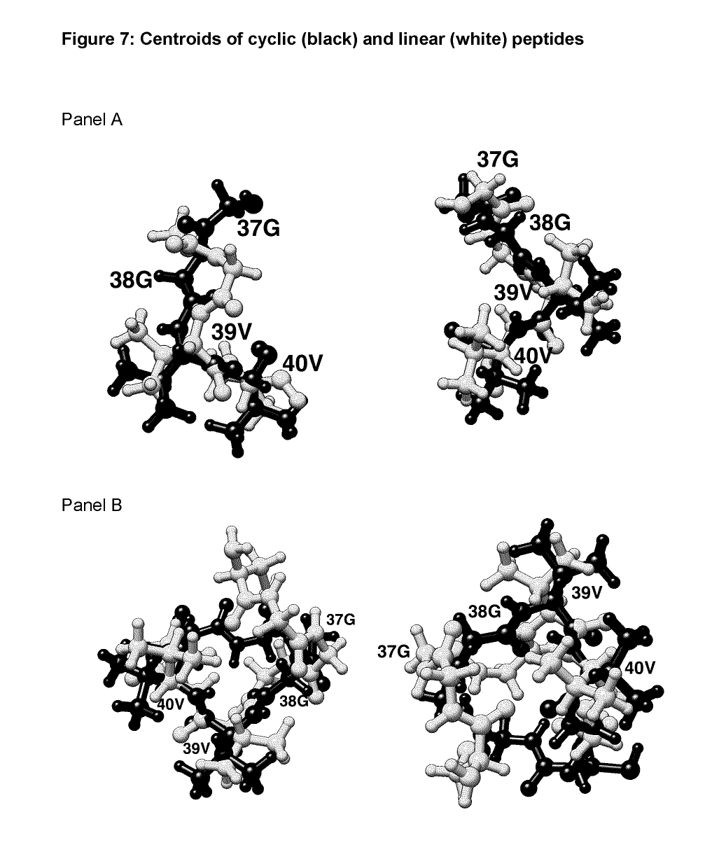

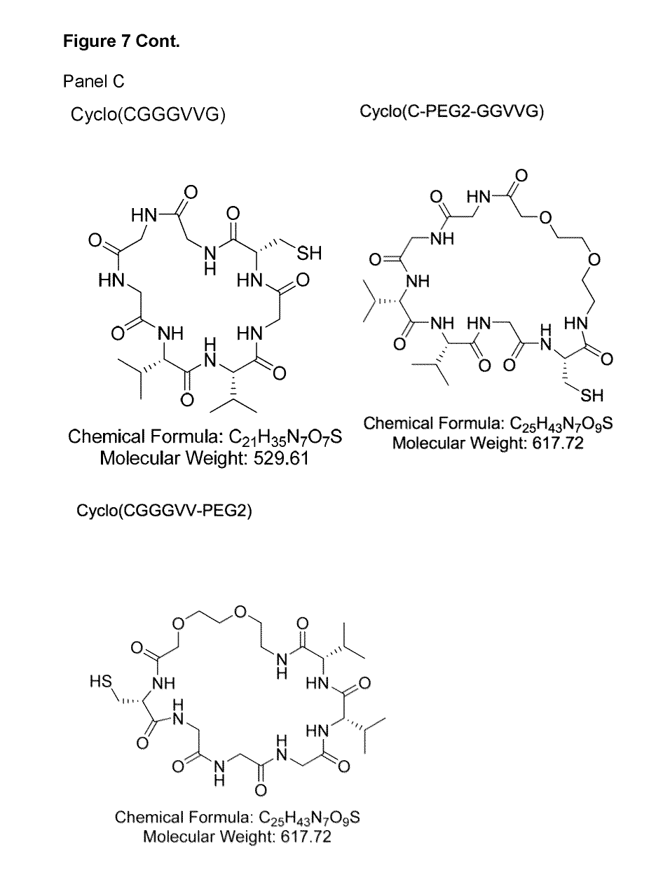

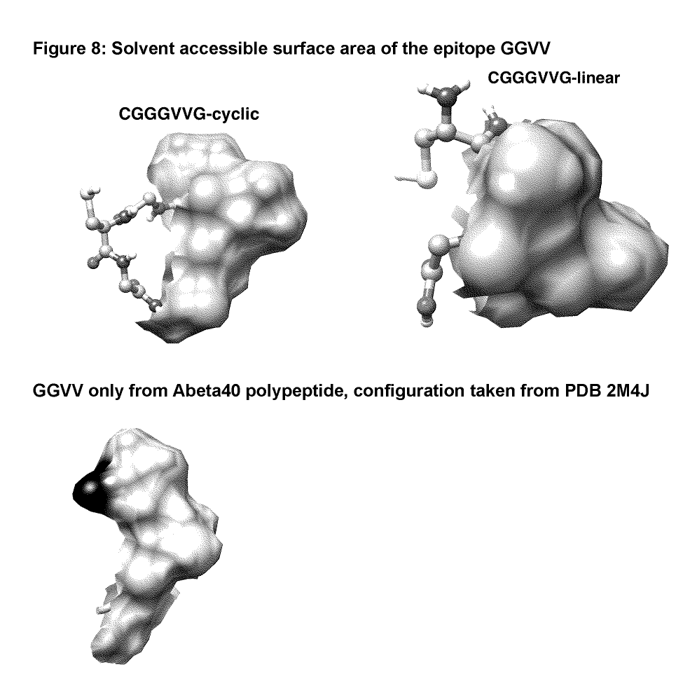

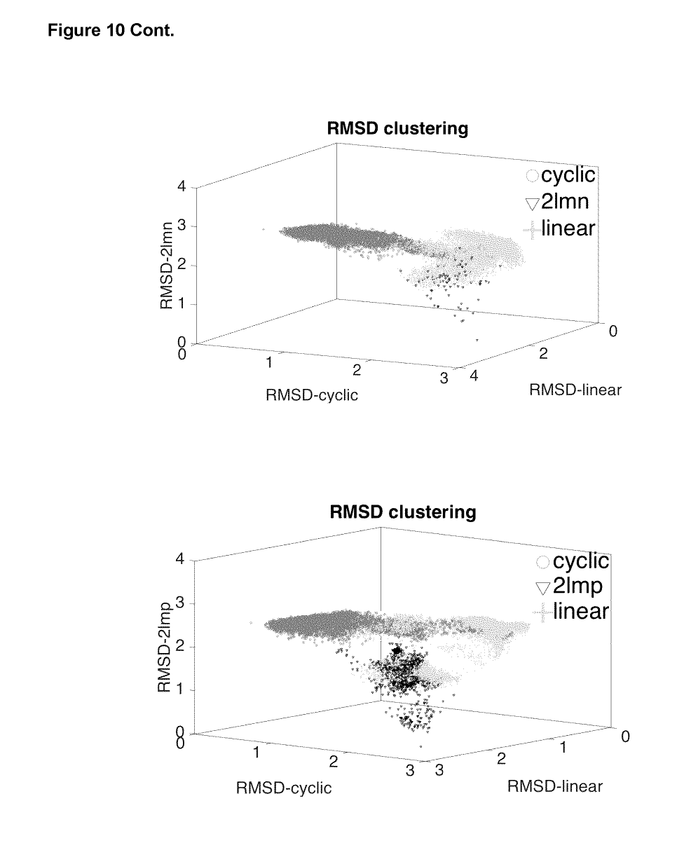

The term “alternate conformation than occupied by G37, G38, V39 and/or V40 in the monomer and/or fibril” as used herein means having one or more differing conformational properties selected from solvent accessibility, charge, entropy, curvature (e.g. in the context of peptide GGVV (SEQ ID NO: 1) as measured for example in the cyclic peptide described in the examples, RMSD structural alignment, and dihedral angle of one or more backbone or side chain dihedral angles compared to said property for 37G, 38G, 39V and/or 40V in A-beta monomer and/or A-beta fibril structures as shown for example in PDBs 2MXU, and shown in FIGS. 1-11 and/or in the Tables. Further, term “alternate conformation than occupied by 37G, 38G, 39 V and/or 40V in the linear peptide” as used herein means having one or more differing conformational properties selected from solvent accessibility, charge, entropy, curvature (e.g. in the context of peptide GGVV (SEQ ID NO: 1) as measured for example in the cyclic peptide described in the examples), RMSD structural alignment, and dihedral angle of one or more backbone or side chain dihedral angles compared to said property for 37G, 38G, 39V and/or 40V in the corresponding linear A-beta peptide or GGVV (SEQ ID NO: 1). For example, FIG. 2 and Table 1 show that the curvature of V39 and V40 in GGVV (SEQ ID NO: 1) in the cyclic peptide ensemble is significantly larger than the curvature of GGVV (SEQ ID NO: 1) in the ensemble of fibril conformations. The curvature for the corresponding residues is also larger in the linear peptide ensemble than it is in the fibril ensemble. Moreover for, the curvature in the cyclic peptide ensemble is substantially higher than that in the linear peptide ensemble for residue V39, while the curvature of V40 in the linear ensemble is higher than the cyclic. The curvature of G37 and G38 in the cyclic ensemble is lower than that in the linear peptide as well, and comparable to that in the fibril. A different curvature profile of the epitope in the cyclic peptide ensemble than either the linear or fibril ensembles implies that conformational selectivity may be conferred, particularly by residues V39 and V40, which exhibit different curvature in the cyclic peptide than either the linear peptide or fibril. Panel A of FIG. 3 shows that the dihedral angle distribution for the angle (O-C-CA-HA1) for G38 in the cyclic peptide ensemble has minimal overlap with the corresponding distributions for the linear peptide and fibril: the overlaps of the linear and fibril distributions with the cyclic distribution are 4.4% and 2.6% respectively. Other dihedral angle distributions had more overlap, however even small differences in individual dihedral distributions can combine to yield globally different antigen profiles. This is elucidated more clearly using cluster analysis of aligned confirmations as described below. FIG. 4G demonstrates that the cyclic peptide is more constrained than the linear peptide, but less than the fibril. FIG. 4F shows that V39 and V40 are more constrained in the cyclic peptide ensemble then they are in the monomer, indicating that the linear monomer will only rarely populate conformations consistent with the cyclic peptide. FIG. 5 demonstrates that the distributions of the Ramachandran dihedral angles for the backbone G38 in the cyclic peptide are substantially different than those for the monomer, and are more similar to those in the fibril. FIG. 6 shows that residues GGVV (SEQ ID NO: 1) have increased solvent accessible surface area, SASA, compared to the fibril, particularly for V39 and V40. FIG. 7 shows that the representative (centroid) structures of the cyclic peptide and linear peptide are distinct. FIG. 8 shows that the surface area profiles of the representative (centroid) structures of the cyclic peptide and linear peptide are distinct. As well, the surface area profile of Abeta40 including charge is distinct from the cyclic surface area profile of GGVV (SEQ ID NO: 1): V40 has a positive charge in Abeta40. FIG. 9 shows that the cyclic peptide equilibrium structures of GGVV (SEQ ID NO: 1) cluster differently than the equilibrium structures of either the linear peptide or corresponding sequence in the fibril 2MXU, while the linear and fibril ensembles are not as clearly differentiated. FIG. 10 shows that this is true for other fibril strains as well.

The term “amino acid” includes all of the naturally occurring amino acids as well as modified L-amino acids. The atoms of the amino acid can for example include different isotopes. For example, the amino acids can comprise deuterium substituted for hydrogen nitrogen-15 substituted for nitrogen-14, and carbon-13 substituted for carbon-12 and other similar changes.

The term “antibody” as used herein is intended to include, monoclonal antibodies, polyclonal antibodies, single chain, veneered, humanized and other chimeric antibodies and binding fragments thereof, including for example a single chain Fab fragment, Fab′2 fragment or single chain Fv fragment. The antibody may be from recombinant sources and/or produced in animals such as rabbits, llamas, sharks etc. Also included are human antibodies that can be produced in transgenic animals or using biochemical techniques or can be isolated from a library such as a phage library. Humanized or other chimeric antibodies may include sequences from one or more than one isotype or class or species.

The phrase “isolated antibody” refers to antibody produced in vivo or in vitro that has been removed from the source that produced the antibody, for example, an animal, hybridoma or other cell line (such as recombinant insect, yeast or bacterial cells that produce antibody). The isolated antibody is optionally “purified”, which means at least: 80%, 85%, 90%, 95%, 98% or 99% purity.

The term “binding fragment” as used herein to a part or portion of an antibody or antibody chain comprising fewer amino acid residues than an intact or complete antibody or antibody chain and which binds the antigen or competes with intact antibody. Exemplary binding fragments include without limitations Fab, Fab′, F(ab′)2, scFv, dsFv, ds-scFv, dimers, nanobodies, minibodies, diabodies, and multimers thereof. Fragments can be obtained via chemical or enzymatic treatment of an intact or complete antibody or antibody chain. Fragments can also be obtained by recombinant means. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be constructed by recombinant expression techniques.

The terms “IMGT numbering” or “ImMunoGeneTics database numbering”, which are recognized in the art, refer to a system of numbering amino acid residues which are more variable (i.e. hypervariable) than other amino acid residues in the heavy and light chain variable regions of an antibody, or antigen binding portion thereof.

When an antibody is said to specifically bind to an epitope such as GGVV (SEQ ID NO:1), what is meant is that the antibody specifically binds to a peptide containing the specified residues or a part thereof for example at least 2 residues of GGVV, with a minimum affinity, and does not bind an unrelated sequence or unrelated sequence spatial orientation greater than for example an isotype control antibody. Such an antibody does not necessarily contact each residue of GGVV (SEQ ID NO:1) and every single amino acid substitution or deletion within said epitope does not necessarily significantly affect and/or equally affect binding affinity.

When an antibody is said to selectively bind an epitope such as a conformational epitope, such as GGVV (SEQ ID NO: 1), what is meant is that the antibody preferentially binds one or more particular conformations containing the specified residues or a part thereof with greater affinity than it binds said residues in another conformation. For example, when an antibody is said to selectively bind a cyclopeptide comprising GGVV or related epitope relative to a corresponding linear peptide, the antibody binds the cyclopeptide with at least a 2 fold greater affinity than it binds the linear peptide.

As used herein, the term “conformational epitope” refers to an epitope where the epitope amino acid sequence has a particular three-dimensional structure wherein at least an aspect of the three-dimensional structure not present or less likely to be present in a corresponding linear peptide is specifically and/or selectively recognized by the cognate antibody. Antibodies which specifically and/or selectively bind a conformation-specific epitope recognize the spatial arrangement of one or more of the amino acids of that conformation-specific/selective epitope. For example an GGVV (SEQ ID NO: 1) conformational epitope, refers to an epitope of GGVV (SEQ ID NO: 1) that is recognized by antibodies specifically and/or selectively, for example at least 2 fold, 3 fold, 5 fold, 10 fold, 50 fold, 100 fold, 250 fold, 500 fold or 1000 fold or greater, more selectively as compared to linear GGVV (SEQ ID NO: 1).

The term “related epitope” as used herein means at least two residues of GGVV (SEQ ID NO: 1) that are antigenic and/or sequences comprising 1or 2 amino acid residues in a A-beta either N-terminal or C-terminal to at least two residues of GGVV (SEQ ID NO: 1). For example it is shown herein GGVV (SEQ ID NO: 1), VGGVV (SEQ ID NO: 6) and GGVVI (SEQ ID NO: 8) were identified as regions prone to disorder in an A-beta fibril. GGVV (SEQ ID NO: 1), VGGVV (SEQ ID NO: 6) and GGVVI (SEQ ID NO: 8) are accordingly related epitopes. Exemplary related epitopes can include epitopes whose sequences are shown in Table 15. The sequences of related epitopes are referred to as “related epitope sequences”.

The term “constrained conformation” as used herein with respect to an amino acid or a side chain thereof, within a sequence of amino acids (e.g. G37 or G38 or V39 or V40 in GGVV (SEQ ID NO: 1)), or with respect to a sequence of amino acids in a larger polypeptide, means decreased rotational mobility of the amino acid dihedral angles, relative to a corresponding linear peptide sequence (e.g. of the linear compound), or the sequence in the context of the larger polypeptide, resulting in a decrease in the number of permissible conformations. This can be quantified for example by finding the entropy reduction for the ensemble of backbone and side chain dihedral angle degrees of freedom, and is plotted in FIG. 4G for each amino acid, for the entropy reduction in the cyclic ensemble and fibril ensemble relative to the linear ensemble. The entropy increase from the fibril ensemble, for both the linear and cyclic peptide ensembles, is plotted in FIGS. 4A-D for the individual dihedral angles in each amino acid. For example, if the side chains in the sequence have less conformational freedom than the linear peptide, the entropy will be reduced. Such conformational restriction would enhance the conformational selectivity of antibodies specifically raised to this antigen.

The term “more constrained conformation” as used herein means that the dihedral angle distribution (ensemble of allowable dihedral angles) of one or more dihedral angles is at least 10% more constrained than in the comparator conformation, as determined for example by the entropy of the amino acids, for example G, and/or V (e.g. a more constrained conformation has lower entropy). Specifically, the percent reduction in entropy as measured by the average entropy change relative to the mean entropy of the linear and cyclic peptides, [(ΔS(cyclic)−ΔS(linear))/(0.5*(ΔS(cyclic)+ΔS(linear))], of GGVV (SEQ ID NO:1) in the overall more constrained cyclic conformational ensemble is on average reduced by more than 10% or reduced by more than 20% or reduced by more than 30% or reduced by more than 40%, from the unconstrained conformational ensemble. The entropy ΔS in the above formula is obtained as the entropy relative to the fibril, e.g. ΔS(cyclic)=S(cyclic)−S(fibril). As an example, the percent reduction in entropy according to the data plotted in FIG. 4F, is 67% for V39 and 31% for V40. G38 also shows an entropy loss of 13%, however G37 actually shows an entropy gain in the cyclic conformation of 61%. The overall entropy reduction of the linear to the cyclic peptide (relative to the fibril entropy) is (ΔS(cyclic)−ΔS(linear))/(0.5*(ΔS(cyclic)+ΔS(linear)))=−27%, i.e. 27% entropy reduction.

The term “no or negligible plaque binding” or “lacks or has negligible plaque binding” as used herein with respect to an antibody means that the antibody does not show typical plaque morphology staining on immunohistochemistry) (e.g. in situ) and the level of staining is comparable to or no more than 2 fold the level seen with an IgG negative (e.g. irrelevant) isotype control

The term “Isolated peptide” refers to peptide that has been produced, for example, by recombinant or synthetic techniques, and removed from the source that produced the peptide, such as recombinant cells or residual peptide synthesis reactants. The isolated peptide is optionally “purified”, which means at least: 80%, 85%, 90%, 95%, 98% or 99% purity and optionally pharmaceutical grade purity.

The term “detectable label” as used herein refers to moieties such as peptide sequences (such a myc tag, HA-tag, V5-tag or NE-tag), fluorescent proteins that can be appended or introduced into a peptide or compound described herein and which is capable of producing, either directly or indirectly, a detectable signal. For example, the label may be radio-opaque, positron-emitting radionuclide (for example for use in PET imaging), or a radioisotope, such as ³H, ¹³N, ¹⁴C, ¹⁸F, ³²F, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion. The detectable label may be also detectable indirectly for example using secondary antibody.

The term “epitope” as commonly used means an antibody binding site, typically a polypeptide segment, in an antigen that is specifically recognized by the antibody. As used herein “epitope” can also refer to the amino acid sequence or a part thereof identified on A-beta using the collective coordinates method described to which antibodies can be raised using a peptide comprising the epitope sequence. For example an antibody generated against an isolated peptide corresponding to a cyclic compound comprising the identified target region GGVV (SEQ ID NO: 1), recognizes part or all of said “epitope” sequence. An epitope is “accessible” in the context of the present specification when it is accessible to binding by an antibody.

The term “greater affinity” as used herein refers to a relative degree of antibody binding where an antibody X binds to target Y more strongly (Kₒₙ) and/or with a smaller dissociation constant (K_{off}) than to target Z, and in this context antibody X has a greater affinity for target Y than for Z. Likewise, the term “lesser affinity” herein refers to a degree of antibody binding where an antibody X binds to target Y less strongly and/or with a larger dissociation constant than to target Z, and in this context antibody X has a lesser affinity for target Y than for Z. The affinity of binding between an antibody and its target antigen, can be expressed as K_{A} equal to 1/K_{D} where K_{D} is equal to kₒₙ/k_{off}. The kₒₙ and k_{off} values can be measured using surface plasmon resonance technology, for example using a Molecular Affinity Screening System (MASS-1) (Sierra Sensors GmbH, Hamburg, Germany). An antibody that is selective for a conformation presented in a cyclic compound optional a cyclic peptide for example has a greater affinity for the cyclic compound (e.g. cyclic peptide) compared to a corresponding sequence in linear form (e.g. the sequence non-cyclized).

Also as used herein, the term “immunogenic” refers to substances which elicit the production of antibodies, activate T-cells and other reactive immune cells directed against an antigenic portion of the immunogen.

The term “corresponding linear compound” with regard to a cyclic compound refers to a compound, optionally a peptide, comprising or consisting of the same sequence or chemical moieties as the cyclic compound but in linear (i.e. non-cyclized) form, for example having properties as would be present in solution of a linear peptide. For example, the corresponding linear compound can be the synthesized peptide that is not cyclized.

As used herein “specifically binds” in reference to an antibody means that the antibody recognizes an epitope sequence and binds to its target antigen with a minimum affinity. For example a multivalent antibody binds its target with a K_{D} of at least 1e-6, at least 1e-7, at least 1e-8, at least 1e-9, or at least 1e-10. Affinities greater than at least 1e-8 may be preferred. An antigen binding fragment such as Fab fragment comprising one variable domain, may bind its target with a 10 fold or 100 fold less affinity than a multivalent interaction with a non-fragmented antibody.

The term “selectively binds” as used herein with respect to an antibody that selectively binds a form of A-beta (e.g. fibril, monomer or oligomer) or a cyclic compound means that the antibody binds the form with at least 2 fold, at least 3 fold, or at least 5 fold, at least 10 fold, at least 100 fold, at least 250 fold, at least 500 fold or at least 1000 fold or more greater affinity. Accordingly an antibody that is more selective for a particular conformation (e.g. oligomer) preferentially binds the particular form of A-beta with at least 2 fold etc., greater affinity compared to another form and/or a linear peptide.

The term “linker” as used herein means a chemical moiety that can be covalently linked to the peptide comprising GGVV (SEQ ID NO: 1) epitope peptide, optionally linked to GGVV (SEQ ID NO: 1) peptide N- and C-termini to produce a cyclic compound. The linker can comprise a spacer and/or one or more functionalizable moieties. The linker via the functionalizable moieties can be linked to a carrier protein or an immunogen enhancing agent such as Keyhole Limpet Hemocyanin (KLH).

The term “spacer” as used herein means any preferably non-immunogenic or poorly immunogenic chemical moiety that can be covalently-linked directly or indirectly to a peptide N- and C-termini to produce a cyclic compound of longer length than the peptide itself, for example the spacer can be linked to the N- and C-termini of a peptide consisting of GGVV (SEQ ID NO:1) to produce a cyclic compound of longer backbone length than the GGVV (SEQ ID NO:1) sequence itself. That is, when cyclized the peptide with a spacer (for example of 3 amino acid residues) makes a larger closed circle than the peptide without a spacer. The spacer may include, but is not limited to, moieties such as G, A, or PEG repeats, e.g. when in combination with the A-beat peptide the sequence being GGGVVG (SEQ ID NO: 9) GGVVG (SEQ ID NO: 10), GGGVV (SEQ ID NO: 11), etc. The spacer may comprise or be coupled to one or more functionalizing moieties, such as one or more cysteine (C) residues, which can be interspersed within the spacer or covalently linked to one or both ends of the spacer. Where a functionalizable moiety such as a C residue is covalently linked to one or more termini of the spacer, the spacer is indirectly covalently linked to the peptide. The spacer can also comprise the functionalizable moiety in a spacer residue as in the case where a biotin molecule is introduced into an amino acid residue.

The term “functionalizable moiety” as used herein refers to a chemical entity with a “functional group” which as used herein refers to a group of atoms or a single atom that will react with another group of atoms or a single atom (so called “complementary functional group”) to form a chemical interaction between the two groups or atoms. In the case of cysteine, the functional group can be —SH which can be reacted to form a disulfide bond. Accordingly the linker can for example be CCC. The reaction with another group of atoms can be covalent or a strong non-covalent bond, for example as in the case as biotin-streptavidin bonds, which can have Kd˜1e-14. A strong non-covalent bond as used herein means an interaction with a Kd of at least 1e-9, at least 1e-10, at least 1e-11, at least 1e-12, at least 1e-13 or at least 1e-14.

Proteins and/or other agents may be functionalized (e.g. coupled) to the cyclic compound, either to aid in immunogenicity, or to act as a probe in in vitro studies. For this purpose, any functionalizable moiety capable of reacting (e.g. making a covalent or non-covalent but strong bond) may be used. In one specific embodiment, the functionalizable moiety is a cysteine residue which is reacted to form a disulfide bond with an unpaired cysteine on a protein of interest, which can be, for example, an immunogenicity enhancing agent such as Keyhole Limpet Hemocyanin (KLH), or a carrier protein such as Bovine serum albumin (BSA) used for in vitro immunoblots or immunohistochemical assays.

The term “reacts with” as used herein generally means that there is a flow of electrons or a transfer of electrostatic charge resulting in the formation of a chemical interaction.

The term “animal” or “subject” as used herein includes all members of the animal kingdom including mammals, optionally including or excluding humans.

A “conservative amino acid substitution” as used herein, is one in which one amino acid residue is replaced with another amino acid residue without abolishing the protein's desired properties. Suitable conservative amino acid substitutions can be made by substituting amino acids with similar hydrophobicity, polarity, and R-chain length for one another. Examples of conservative amino acid substitution include:

Conservative Substitutions

| Type of Amino Acid | Substitutable Amino Acids |
|---|---|
| Hydrophilic | Ala, Pro, Gly, Glu, Asp, Gln, Asn, Ser, Thr |
| Sulphydryl | Cys |
| Aliphatic | Val, Ile, Leu, Met |
| Basic | Lys, Arg, His |
| Aromatic | Phe, Tyr, Trp |

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

For antibodies, percentage sequence identities can be determined when antibody sequences maximally aligned by IMGT or other (e.g. Kabat numbering convention). After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

The term “nucleic acid sequence” as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid can be either double stranded or single stranded, and represents the sense or antisense strand. Further, the term “nucleic acid” includes the complementary nucleic acid sequences as well as codon optimized or synonymous codon equivalents. The term “isolated nucleic acid sequences” as used herein refers to a nucleic acid substantially free of cellular material or culture medium when produced by recombinant DNA techniques, or chemical precursors, or other chemicals when chemically synthesized. An isolated nucleic acid is also substantially free of sequences which naturally flank the nucleic acid (i.e. sequences located at the 5′ and 3′ ends of the nucleic acid) from which the nucleic acid is derived.

“Operatively linked” is intended to mean that the nucleic acid is linked to regulatory sequences in a manner which allows expression of the nucleic acid. Suitable regulatory sequences may be derived from a variety of sources, including bacterial, fungal, viral, mammalian, or insect genes. Selection of appropriate regulatory sequences is dependent on the host cell chosen and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

The term “vector” as used herein comprises any intermediary vehicle for a nucleic acid molecule which enables said nucleic acid molecule, for example, to be introduced into prokaryotic and/or eukaryotic cells and/or integrated into a genome, and include plasmids, phagemids, bacteriophages or viral vectors such as retroviral based vectors, Adeno Associated viral vectors and the like. The term “plasmid” as used herein generally refers to a construct of extrachromosomal genetic material, usually a circular DNA duplex, which can replicate independently of chromosomal DNA.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6(Log 10 [Na+])+0.41(%(G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5× Denhardt's solution/1.0% SDS at Tm−5° C. based on the above equation, followed by a wash of 0.2×SSC/0.1% SDS at 60° C. Moderately stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 2002, and in: Sambrook et al., Molecular Cloning: a Laboratory Manual, Cold Spring Harbor Laboratory Press, 2001.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with early stage AD can be treated to prevent progression can be treated with a compound, antibody, immunogen, nucleic acid or composition described herein to prevent progression.

The term “administered” as used herein means administration of a therapeutically effective dose of a compound or composition of the disclosure to a cell or subject.

As used herein, the phrase “effective amount” means an amount effective, at dosages and for periods of time necessary to achieve a desired result. Effective amounts when administered to a subject may vary according to factors such as the disease state, age, sex, weight of the subject. Dosage regime may be adjusted to provide the optimum therapeutic response.

The term “pharmaceutically acceptable” means that the carrier, diluent, or excipient is compatible with the other components of the formulation and not substantially deleterious to the recipient thereof.

Compositions or methods “comprising” or “including” one or more recited elements may include other elements not specifically recited. For example, a composition that “comprises” or “includes” an antibody may contain the antibody alone or in combination with other ingredients.

In understanding the scope of the present disclosure, the term “consisting” and its derivatives, as used herein, are intended to be close ended terms that specify the presence of stated features, elements, components, groups, integers, and/or steps, and also exclude the presence of other unstated features, elements, components, groups, integers and/or steps.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” Further, it is to be understood that “a,” “an,” and the include plural referents unless the content clearly dictates otherwise. The term “about” means plus or minus 0.1 to 50%, 5-50%, or 10-40%, preferably 10-20%, more preferably 10% or 15%, of the number to which reference is being made.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The singular forms of the articles “a,” “an,” and “the” include plural references unless the context clearly dictates otherwise. For example, the term “a compound” or “at least one compound” can include a plurality of compounds, including mixtures thereof.

### II. Epitopes and Binding Proteins

The inventors have identified an epitope in A-beta, comprising GGVV (SEQ ID NO:1) at amino acids 37 to 40 on A-beta. They have further identified that the epitope or a part thereof may be a conformational epitope, and that GGVV (SEQ ID NO:1) may be selectively accessible to antibody binding in oligomeric species of A-beta.

Without wishing to be bound by theory, fibrils may present interaction sites that have a propensity to catalyze oligomerization. This may be strain-specific, and may only occur when selective fibril surface not present in normal individuals is exposed and thus able to have aberrant interactions with the monomer (i.e. is presented to the monomer). Environmental challenges such as low pH, osmolytes present during inflammation, or oxidative damage may induce disruption in fibrils that can lead to exposure of more weakly stable regions. There is interest, then, to predict these weakly-stable regions, and use such predictions to rationally design antibodies that could target them. In addition regions likely to be disrupted in the fibril may also be good candidates for exposed regions in oligomeric species.

Computer based systems and methods to predict contiguous protein regions that are prone to disorder are described in U.S. Patent Application Ser. No. 62/253,044, SYSTEMS AND METHODS FOR PREDICTING MISFOLDED PROTEIN EPITOPES BY COLLECTIVE COORDINATE BIASING filed Nov. 9, 2015, and U.S. patent application Ser. No. 12/574,637, “METHODS AND SYSTEMS FOR PREDICTING MISFOLDED PROTEIN EPITOPES” filed Oct. 6, 2009, each of which is hereby incorporated by reference in its entirety. As described in the Examples, the methods were applied to A-beta and identified an epitope that as demonstrated herein is specifically or selectively more accessible in A-beta oligomers.

As described in the Examples, cyclic peptide cyclo(CGGGVVG) (SEQ ID NO: 2) may capture or of more of the conformational differences of the GGVV (SEQ ID NO: 1) epitope in oligomers relative to the monomer and/or fibril species. For example, differences in solvent accessible surface, curvature, RMSD structural alignment, and the dihedral angle distributions for amino acids and dihedral angles in the cyclic 7-mer cyclo (CGGGVVG) (SEQ ID NO: 2) were found to be substantially different than either the monomer and/or fibril, suggesting that the cyclic peptide provides for a conformational epitope that is distinct from the linear peptide. Antibodies raised using an immunogen comprising cyclo(CGGGVVG) (SEQ ID NO: 2) selectively bound cyclo(CGGGVVG) (SEQ ID NO: 2) over linear CGGGVVG (SEQ ID NO: 2) and selectively bound synthetic and/or native oligomeric A-beta species compared to monomeric A-beta and A-beta fibril plaques. Further antibodies raised to cyclo(CGGGVVG) (SEQ ID NO: 2) were able to inhibit in vitro propagation of A-beta aggregation. In addition, as demonstrated in a toxicity assay, antibodies raised against (CGGGVVG) (SEQ ID NO: 2) inhibited A-beta oligomer neural cell toxicity.

### a). GGVV (SEQ ID NO:1) “Epitope” Compounds

Accordingly, the present disclosure identifies a conformational epitope in A-beta consisting of amino acids GGVV (SEQ ID NO: 1) or a part thereof such as GVV, GGVV (SEQ ID NO: 1) corresponding to amino acids residues 37-40 on A-beta. As demonstrated in the Examples, epitopes GGVV (SEQ ID NO: 1), GGVVI (SEQ ID NO: 8) and VGGVV (SEQ ID NO: 6), (included in the epitopes collectively referred to herein as GGVV (SEQ ID NO: 1) and related epitopes) were identified as regions prone to disorder in an A-beta fibril. The residues GGVV (SEQ ID NO: 1) emerged in two predictions using the collective coordinates method, while the flanking residues of this epitope, 36V and 41I, each occurred in one prediction. The residues GGVV (SEQ ID NO: 1) also emerged using the Promis Gō method.

An aspect includes a compound comprising an A-beta peptide comprising or consisting of GGVV (SEQ ID NO: 1), a related epitope sequence including part of any of the foregoing, wherein if the peptide is GGVV(SEQ ID NO: 1), the peptide is in a conformation that is distinct in at least one feature from linear GGVV (SEQ ID NO: 1), for example the terminal valine is covalently bound to an amino acid or other moiety through its carboxyl terminus and is therefore uncharged. For example, in a cyclic conformation the C terminal valine will due to cyclization not comprise the carboxylate negative charge.

In an embodiment, the A-beta peptide is selected from an amino acid sequence comprising or consisting of GGVV (SEQ ID NO: 1), VGGVV (SEQ ID NO: 6) or GGVVI (SEQ ID NO: 8). In an embodiment, the A-beta peptide has a sequence as set forth in any one of the A-beta sequences set forth in Table 14.

In an embodiment, the compound is a cyclic compound, such as a cyclopeptide. The terms cyclopeptide and cyclic peptide are used interchangeably herein.

In some embodiments, the A-beta peptide, optionally a conformational peptide, comprising GGVV (SEQ ID NO: 1) (or a part thereof) or a related epitope sequence can include 1, or 2 additional residues in A-beta N- and/or C-terminus of GGVV (SEQ ID NO: 1) (or a part thereof) for example the A-beta peptide can include 1 residue C-terminal and be VGGVV (SEQ ID NO: 6). As shown for example in the A-beta sequence of SEQ ID NO: 16, the 3 amino acids N-terminal to GGVV (SEQ ID NO: 1) in A-beta are LMV and the 2 amino acids C-terminal to GGVV (SEQ ID NO:1) in forms A-beta 1-42 and 1-43 are IA. In embodiments where the compound comprising the A-beta peptide is cyclized, the A-beta peptide is or is a maximum of 6 A-beta residues. In an embodiment, the A-beta peptide is or is a maximum of 5 A-beta residues. In yet another embodiment A beta peptide (e.g. in the compound such as a cyclic compound) is 4 A-beta residues, optionally GGVV (SEQ ID NO: 1).

In an embodiment, the compound further includes a linker. The linker comprises a spacer and/or one or more functionalizable moieties. The linker can for example comprise 1, 2, 3, 4, 5, 6, 7 or 8 amino acids and/or equivalently functioning molecules such as polyethylene glycol (PEG) moieties, and/or a combination thereof. In an embodiment, the spacer amino acids are selected from non-immunogenic or poorly immunogenic amino acid residues such as G and A, for example the spacer can be GGG, GAG, G(PEG)G, PEG-PEG (also referred to as PEG2)-GG and the like. One or more functionalizable moieties e.g. amino acids with a functional group may be included for example for coupling the compound to an agent or detectable label or a carrier such as BSA or an immunogenicity enhancing agent such as KLH.

In an embodiment the linker comprises GC-PEG, PEG-GC, GCG or PEG2-CG.

In an embodiment, the linker comprises 1, 2, 3, 4, 5, 6, 7 or 8 amino acids.

In certain embodiments, the cyclic compound has a maximum of 12, 11, 10, 9, 8, or 7 residues, optionally amino acids and/or equivalent units such as PEG units or other similar sized chemical moieties.

In embodiments wherein the A-beta peptide comprising GGVV (SEQ ID NO: 1) or a part thereof or a related epitope sequence includes 1, 2 or 3 additional residues found in A-beta that are N- and/or C-terminal to GGVV (SEQ ID NO: 1) the linker is covalently linked to the N- and/or C-termini of the A-beta residues (e.g. where the peptide is VGGVV (SEQ ID NO: 6), the linker is covalently linked to R and G residues). Similarly, where the A-beta peptide is GGVV (SEQ ID NO: 1), the linker is covalently linked to residues G and GV and where the A-beta peptide is GGVVI (SEQ ID NO: 8), the linker is covalently linked to residues G and I.

Proteinaceous portions of compounds (or the compound wherein the linker is also proteinaceous) may be prepared by chemical synthesis using techniques well known in the chemistry of proteins such as solid phase synthesis or synthesis in homogenous solution.

As mentioned, the compound can be a cyclic compound. Reference to the “cyclic peptide” herein can refer to a fully proteinaceous compound (e.g. wherein the linker is for example 1, 2, 3, 4, 5, 6, 7 or 8 amino acids). It is understood that properties described for the cyclic peptide determined in the examples can be incorporated in other compounds (e.g. cyclic compounds) comprising non-amino acid linker molecules.

An aspect therefore provides a cyclic compound comprising peptide GGVV (SEQ ID NO: 1) (or a part thereof such as GGV) or a related epitope sequence and a linker, wherein the linker is covalently coupled directly or indirectly to the peptide comprising GGVV (SEQ ID NO: 1) (e.g. the G and the V residues when the peptide consists of GGVV (SEQ ID NO: 1)). In the cyclic compound for example, at least the one of the G and/or V residues is in an alternate conformation than the G and/V residues in a linear compound, optionally a corresponding linear peptide, optionally in a more constrained conformation.

In an embodiment, the cyclic compound comprises an A-beta peptide comprising GGVV (SEQ ID NO: 1) or a related epitope sequence and up to 6 A-beta residues (e.g. 1 or 2 amino acids N and/or C terminus to GGVV (SEQ ID NO: 1)) and a linker, wherein the linker is covalently coupled directly or indirectly to the peptide N-terminus residue and the C-terminus residue of the A-beta peptide. In the cyclic compound for example at least V is in an alternate conformation than V in a corresponding linear peptide or at least G is in an alternate conformation than G in a corresponding linear peptide and optionally wherein at least G, or at least V, is in a more constrained conformation than the conformation occupied in a linear compound, optionally the corresponding linear peptide comprising GGVV (SEQ ID NO: 1).

The linear peptide comprising the A-beta sequence can be comprised in a linear compound. The linear compound or the linear peptide comprising GGVV (SEQ ID NO: 1) is in an embodiment, a corresponding linear peptide. In another embodiment, the linear peptide is any length of A-beta peptide comprising GGVV (SEQ ID NO: 1), including for example a linear peptide comprising A-beta residues 10-42, or smaller portions thereof such as A-beta residues 20-42, 20-40, 30-42 and the like etc. optionally comprising linker sequence. The linear peptide can in some embodiments also be a full length A-beta peptide.

In an embodiment, the cyclic compound comprises the sequence of any one of SEQ ID NOS: 2-4.

The cyclic compound can be synthesized as a linear molecule with the linker covalently attached to the N-terminus or C-terminus of the peptide comprising the A-beta peptide, optionally GGVV (SEQ ID NO: 1) or related epitope, prior to cyclization. Alternatively part of the linker is covalently attached to the N-terminus and part is covalently attached to the C-terminus prior to cyclization. In either case, the linear compound is cyclized for example in a head to tail cyclization (e.g. amide bond cyclization).

In an embodiment the cyclic compound comprises an A-beta peptide comprising or consisting of GGVV (SEQ ID NO: 1) and a linker, wherein the linker is coupled to the N- and C-termini of the peptide (e.g. the G and the V residues when the peptide consists of GGVV (SEQ ID NO: 1). In an embodiment, at least one of the G and/or V residues is in an alternate conformation in the cyclic compound than occupied by at least one of the G and/or V residues in a linear peptide comprising GGVV (SEQ ID NO: 1).

In an embodiment, at least one of the G and/or V residues is in an alternate conformation in the cyclic compound than occupied by a residue, optionally by G and/or V, in the monomer and/or fibril.

In an embodiment, at least one of the G and/or V residues is in an alternate conformation in the cyclic compound than occupied by a residue in the monomer and/or fibril.

In an embodiment, the alternate conformation is a constrained conformation.

In an embodiment, at least a V, optionally alone or in combination with at least a second V, is in a more constrained conformation than the conformation occupied in a linear peptide comprising GGVV (SEQ ID NO: 1).

In an embodiment, the conformation of G and/or V in combination with one or more of G and/or V is comprised in the compound in an alternate conformation, optionally in a more constrained conformation.

For example, the alternate conformation can include one or more differing dihedral angles in residue G38 differing from the dihedral angles in the linear peptide and/or peptide in the context of the fibril.

In an embodiment, the cyclic compound comprises a minimum average side-chain/backbone dihedral angle difference between the cyclic compound and linear peptide.

In an embodiment, the cyclic compound comprises a residue selected from G and V, wherein at least one dihedral angle is at least 30 degrees, at least 40 degrees, at least 50 degrees, at least 60 degrees, at least 70 degrees, at least 80 degrees, at least 90 degrees, at least 100 degrees, at least 110 degrees, at least 120 degrees, at least 130 degrees, at least 140 degrees or at least 150 degrees different in the cyclic compound, than the corresponding dihedral angle in the context of the linear or the fibril compound.

In an embodiment, the G is G38 and the V is V40.

As shown in FIG. 3, the dihedral angle distribution of G38 and also V40 is substantially different in the cyclic peptide compared to the linear peptide, or the residue in the context of the fibril 2MXU. For example, Table 3 indicates that for simulated linear peptides, cyclic peptides, and fibrils, the difference in the dihedral angle O-C-CA-N of G38 is most likely about −172.5 degrees between cyclic and linear, and about 40.0 degrees between cyclic and fibril. In an embodiment, the cyclic compound comprises a G residue comprising an O-C-Cα-N (also referred to as O-C-CA-N) dihedral angle that is at least 30 degrees, at least 40 degrees, at least 50 degrees, at least 60 degrees, at least 70 degrees, at least 80 degrees, at least 90 degrees, at least 100 degrees, at least 110 degrees, at least 120 degrees, at least 130 degrees, at least 140 degrees, or at least 150 degrees different, than the corresponding dihedral angle in the context of the linear peptide and/or fibril. Similarly, t differences in dihedral angles between cyclic and linear peptides for G38 dihedrals O-C-CA-H1, O-C-CA-H2 are most likely about 60 and 55 degrees respectively. Accordingly in an embodiment, the cyclic compound comprises a G comprising a dihedral angle O-C-Cα-H1 and/or O-C-Cα-H2 that is at least 20 degrees different, at least 30 degrees different, or at least 40 degrees different than the corresponding dihedral angle in the context of the linear compound. The corresponding differences in most-likely dihedral angles between cyclic peptide and fibril peptides for G38 dihedrals O-C-CA-H1, O-C-CA-H2 are 91 and 160 degrees respectively. Accordingly in an embodiment, the cyclic compound comprises a G comprising dihedral angle for O-C-Cα-H1 and/or O-C-Cα-H2 that is at least 20 degrees different, at least 30 degrees different, at least 40 degrees different, at least 50 degrees different, at least 60 degrees different, at least 70 degrees different, at least 80 degrees different, at least 90 degrees different, at least 100 degrees different, at least 110 degrees different, at least 120 degrees different, at least 130 degrees different, at least 140 degrees different, or at least 150 degrees different, than the corresponding dihedral angle in the context of the fibril.

Table 3 also identifies differences in the dihedral angle distributions for other angles, including those for example in residues 38G and 40V.

According to the peak values of Ramachandran angles given in Table 5, the most-likely Ramachandran ψ values are different between the cyclic and linear peptides. The linear peptide displays 4 peak values; the cyclic peptide displays 2 peak values. There are then 8 corresponding differences Δψ between peak values for the linear and cyclic peptides: 175, 170, 175, 175, 170, 175, 160, and 170 degrees. The Δψ values are substantially different between the linear and cyclic peptides.

Table 3 also describes differences in dihedral angles for V40. The dihedral angles distribution of V40 O-C-CA-CB has two peaks for cyclic and linear; the dihedral angle difference for V40 O-C-CA-CB between the cyclic and linear peptides are most likely about 15 degrees and 30 degrees. The fibril distribution has one peak. The differences in the V40 O-C-CA-CB dihedral are, when comparing between the cyclic and fibril is most likely about 10 degrees and 170 degrees.

In an embodiment, the cyclic compound comprises a V comprising an dihedral angle O-C-CA-CB that is at least 10 degrees, at least 20 degrees different, at least 30 degrees, at least 40 degrees, at least 50 degrees, at least 60 degrees, at least 70 degrees, at least 80 degrees, at least 90 degrees, at least 100 degrees, at least 110 degrees, at least 120 degrees, at least 130 degrees, at least 140 degrees or at least 150 degrees different than the corresponding dihedral angle in the context of the linear compound and/or the fibril compound.

The angle difference can for example be positive or negative, (+) or (−).

The alternate conformation can comprise an alternate backbone orientation. For example, the backbone orientation that the cyclic epitope exposes for an antibody differs compared to linear or fibril form.

FIG. 5 plots the backbone phi and psi angles sampled in equilibrium simulations, for residue 38G in both linear and cyclic peptides consisting of sequence CGGGVVG (SEQ ID NO: 2), as well as GGVV (SEQ ID NO: 1) in the context of the equilibrated fibril structure using initial condition from PDB 2MXU. From FIG. 5 it is seen that the distribution of backbone dihedral angles (Ramachandran phi/psi angles) in the cyclic peptide is different from the distribution of Ramachandran angles sampled for the linear peptide, and more similar to the peptide GGVV (SEQ ID NO: 1) in the context the fibril structure 2MXU, for residue G38 . Table 5 lists peak values of distributions of backbone phi/psi angles, while Table 4 lists overlap between the distributions of backbone phi/psi angles. The overlap between the cyclic and linear distributions is low: the linear distribution overlaps with the cyclic by about 16%. Moreover, for the centroid conformations of the largest cyclic cluster, largest linear cluster, and largest fibril cluster (centroid structures are shown in FIG. 7 and FIG. 8), the Ramachandran angles for G38 are (ϕ,ψ)=(−75.4, −174.3), (ϕ,ψ)=(71.6,9.9), and (ϕ,ψ)=(98.0, 116.9) respectively. Thus the backbone Ramachandran angle difference (Δϕ,Δψ)=(−147, 175.8) between the cyclic and linear peptide for G38 in GGVV (SEQ ID NO: 1), and the backbone Ramachandran angle difference (Δϕ,Δψ)=(−173.4, 68.8) between the cyclic and fibril peptide for G38 in GGVV (SEQ ID NO: 1). This suggests that the representative structures have different backbone dihedral angle.

Accordingly, in an embodiment, the cyclic compound comprises an A-beta peptide with at least one residue wherein backbone phi/psi angles is at least 20, at least 30 degrees, at least 40 degrees, at least 50 degrees, at least 60 degrees, at least 70 degrees, at least 80 degrees different, at least 90 degrees, at least 100 degrees, at least 110 degrees, at least 120 degrees, at least 130 degrees, at least 140 degrees, at least 150 degrees compared to a linear compound, optionally the corresponding linear peptide or in a fibril PDB structure.

The alternate conformation can also include an increase in or decrease in curvature centered around an amino acid or of the cyclic compound comprising GGVV (SEQ ID NO:1) or a related epitope relative to a linear compound, optionally a corresponding linear peptide and/or A-beta fibril.

In an embodiment, the alternate conformation GGVV (SEQ ID NO: 1) has altered curvature profile relative to linear GGVV (SEQ ID NO: 1), or GGVV (SEQ ID NO: 1) in the context of the fibril structure 2MXU. As shown in FIG. 2G for which numbers are given in Table 1, the curvature V39 in the cyclic peptide is substantially larger than the values in either the linear peptide or fibril. The curvature of cyclic V40 is intermediate between the fibril and monomer.

The values of the curvature were determined for from N- to C-terminus G, G, V and V in cyclo (CGGGVVG) (SEQ ID NO: 2), linear CGGGVVG (SEQ ID NO: 2), and GGVV (SEQ ID NO: 1) in the context of the fibril and are described in Example 2.

Accordingly, the compound comprises an A-beta peptide wherein the curvature of the V39 in the alternate conformation is increased by at least 0.1, 0.2, 0.3 or more radians compared to the corresponding linear peptide in the context of the fibril.

In an embodiment, the terminal V, GG, GV, VV, GGV, VVG, and/or GGVV (SEQ ID NO: 1) are in an alternate conformation, for example as compared to what is occupied by these residues in a non-oligomeric conformation, such as the linear peptide and/or fibril.

FIG. 2A plots the curvature for linear CGGGVVG (SEQ ID NO:2) as obtained from different equilibrium simulation times. The legend shows several curves that start from 10 ns and continue to either 30 ns, 50 ns, 70 ns, or 90 ns. As simulation time is increased, the curvature values converge to the values reported above and in Table 1. Similar studies are shown in FIG. 2B for the cyclic peptide and FIG. 2C for the fibril. Panels D, E, and F show the convergence in the sum of the curvature values as a function of simulation time, for the linear, cyclic, and fibril conformations respectively. The degree of convergence indicates that the error bars are approximately 0.016 radian for the cyclic peptide, 0.05 radian for the linear peptide, and 0.01 radian for the fibril.

Cyclic compounds which show similar changes are also encompassed.

The cyclic compound in some embodiments that comprises a peptide comprising GGVV (SEQ ID NO: 1) can include 1, or 2 or more residues in A-beta upstream and/or downstream of GGVV (SEQ ID NO: 1). In such cases the spacer is covalently linked to the N- and C-termini of the ends of the corresponding residues of the A-beta sequence.

In some embodiments, the linker or spacer is indirectly coupled to the N- and C-terminus residues of the A-beta peptide.

In an embodiment, the cyclic compound is a compound in FIG. 7C.

Methods for making cyclized peptides are known in the art and include SS-cyclization or amide cyclization (head-to-tail, or backbone cyclization). Methods are further described in Example 3. For example, a peptide with “C” residues at its N- and C-termini, e.g. CGGGVVGC (SEQ ID NO: 12), can be reacted by SS-cyclization to produce a cyclic peptide. As described in Example 2, a cyclic compound of FIG. 7C was assessed for its relatedness to the conformational epitope identified. The cyclic compound comprising GGVV (SEQ ID NO: 1) peptide for example can be used to raise antibodies selective for one or more conformational features.

The epitope GGVV (SEQ ID NO: 1) and/or a part thereof, as described herein may be a potential target in misfolded propagating strains of A-beta involved in A-beta, and antibodies that recognize the conformational epitope may for example be useful in detecting such propagating strains.

Also provided in another aspect is an isolated peptide comprising an A-beta peptide sequence described herein, including linear peptides and cyclic peptides. Linear peptides can for example be used for selecting antibodies for lack of binding thereto. The isolated peptide can comprise a linker sequence described herein. The linker can be covalently coupled to the N or C terminus or may be partially coupled to the N terminus and partially coupled to the C terminus as in CGGGVVG (SEQ ID NO: 2) linear peptide. In the cyclic peptide, the linker is coupled to the C-terminus and N-terminus directly or indirectly.

Another aspect includes an immunogen comprising a compound, optionally a cyclic compound described herein. The immunogen may also comprise additional A-beta sequence. The amino acids may be directly upstream and/or downstream (i.e. N-terminal and/or C-terminal) of the GGVV (SEQ ID NO: 1) and related epitope sequence. Antibodies raised against such immunogens can be selected for example for binding to a cyclopeptide comprising GGVV (SEQ ID NO:1) or a related epitope.

An immunogen is suitably prepared or formulated for administration to a subject, for example, the immunogen may be sterile, or purified. In an embodiment, the immunogen is a cyclic peptide comprising GGVV (SEQ ID NO: 1) or a related epitope sequence.

In an embodiment, the immunogen comprises immunogenicity enhancing agent such as Keyhole Limpet Hemocyanin (KLH) or a MAP antigen. The immunogenicity enhancing agent can be coupled to the compound either directly, such as through an amide bound, or indirectly through a functionalizable moiety in the linker. When the linker is a single amino acid residue (for example with the A-beta peptide in the cyclic compound is 6 amino acid residues) the linker can be the functionalizable moiety (e.g. a cysteine residue).

The immunogen can be produced by conjugating the cyclic compound containing the constrained epitope peptide to an immunogenicity enhancing agent such as Keyhole Limpet Hemocyanin (KLH) or a carrier such bovine serum albumin (BSA)using for example the method described in Lateef et al 2007, herein incorporated by reference. In an embodiment, the method described in Example 3 or 4 is used.

A further aspect is an isolated nucleic acid molecule encoding the proteinaceous portion of a compound or immunogen described herein.

In embodiment, the nucleic acid molecule encodes any one of the amino acid sequences sent forth in SEQ ID NOS: 1-15.

In an embodiment, nucleic acid molecule encodes GGVV (SEQ ID NO: 1) or a related epitope and optionally a linker described herein.

A further aspect is a vector comprising said nucleic acid. Suitable vectors are described elsewhere herein.

### b) Antibodies, Cells and Nucleic Acids

As demonstrated in Examples 6 and 7, the cyclic compound CGGGVVG (SEQ ID NO: 2) was immunogenic, and produced a number of antibodies that selectively bind the cyclic compound relative to a linear compound, optionally the corresponding linear peptide. As described herein, antibodies raised using cyclo(CGGGVVG) (SEQ ID NO: 2) included antibodies that were selective for the cyclic compound, selectively bound A-beta oligomer over monomer, and lacked appreciable plaque staining in AD tissue. The epitope GGVV (SEQ ID NO: 1) and/or a part thereof, as described herein may be a potential target in misfolded propagating strains of A-beta involved in A-beta, and antibodies that recognize the conformational epitope may for example be useful in detecting such propagating strains. Further antibodies raised to the cyclic compound inhibited A-beta aggregation and also inhibited A-beta oligomer induced neural cell toxicity suggesting their use as therapeutics.

Accordingly, the compounds and particularly the cyclic compounds described above can be used to raise antibodies that specifically bind VV, GVV, and/or GGVV (SEQ ID NO: 1) in A-beta and/or which recognize specific conformations of these residues in A-beta, including one or more differential features described herein. Similarly cyclic compounds comprising for example VGGVV (SEQ ID NO: 6), GGVVI (SEQ ID NO: 8), VGGVVI (SEQ ID NO: 7) and/or other related epitope sequences described herein can be used to raise antibodies that specifically bind GGVV (SEQ ID NO: 1) etc and/or specific conformational epitopes thereof.

Accordingly as aspect includes an antibody (including a binding fragment thereof) that specifically binds to an A-beta peptide having a sequence of GGVV (SEQ ID NO: 1) or a related epitope sequence, for example an A-beta sequence as set forth in any one of SEQ ID NO: 1 to 15.

In an embodiment, the A-beta peptide is comprised in a cyclic peptide and the antibody is specific and/or selective for A-beta presented in the cyclic compound.

In an embodiment, the antibody specially and/or selectively binds the A-beta peptide of acyclic compound described herein, wherein the A-beta has an A-beta sequence as set forth in any one of SEQ ID NOs: 1 to 15, for example one of SEQ ID Nos 1, and 5-15. In an embodiment the cyclic compound comprises a sequence as set forth in any one of SEQ ID Nos: 2-4.

In an embodiment, the cyclic compound is a cyclic peptide. In an embodiment, A-beta peptide in the cyclic peptide is the A-beta peptide of any one of SEQ ID NO: 1-15 for example one of SEQ ID Nos 1 and 5-15.

In an embodiment, the antibody is produced using a cyclic compound of any one of SEQ ID Nos: 2-4 or an immunogen comprising said compound.

As described in the examples, antibodies having one or properties can be selected using assays described in the Examples.

In an embodiment, the antibody does not bind a linear peptide comprising the sequence GGVV (SEQ ID NO: 1), optionally wherein the sequence of the linear peptide is a linear version of a cyclic sequence used to raise the antibody, optionally as set forth in SEQ ID NO: 2.

In an embodiment, the antibody is selective for the A-beta peptide as presented in the cyclic compound relative to a corresponding linear compound comprising the A-beta peptide.

In an embodiment, the antibody specifically binds an epitope on A-beta, the epitope comprising or consisting of GGVV (SEQ ID NO: 1) or a related epitope thereof.

In an embodiment, the epitope recognized specifically or selectively by the antibody on A-beta is a conformational epitope.

In an embodiment the antibody is isolated.

In an embodiment, the antibody is an exogenous antibody.

In an embodiment, the antibody does not specifically bind and/or is not selective for linear AIIGLMVGGVV (SEQ ID NO: 13), linear MVGGVV (SEQ ID NO: 14) or linear GVVIA (SEQ ID NO: 15) relative to cyclic compound comprising a peptide consisting of GGVV (SEQ ID NO: 1). Selectively can be measured using an ELISA as described herein.

Accordingly a further aspect is an antibody which specifically binds an epitope present on A-beta, wherein the epitope comprises or consists of at least one amino acid residue predominantly involved in binding to the antibody, wherein the at least one amino acid is G or V embedded within the sequence GGVV (SEQ ID NO: 1), wherein the epitope when consisting of GGVV (SEQ ID NO: 1) is a conformational epitope (e.g selectively binds a peptide in an alternate optionally constrained conformation relative to a linear compound, optionally the corresponding linear peptide, for example where at least one amino acid of the epitope is more constrained). In an embodiment, the epitope comprises or consists of at least two consecutive amino acid residues predominantly involved in binding to the antibody, wherein the at least two consecutive amino acids are GG or GV or VV embedded within GGVV (SEQ ID NO: 1).

In another embodiment, the epitope consists of GGVV (SEQ ID NO: 1) or a related epitope.

In another embodiment, the epitope is a conformational epitope and consists of GGVV (SEQ ID NO: 1). In an embodiment, the antibody selectively binds GGVV (SEQ ID NO: 1) in a cyclic peptide, optionally cyclo(CGGGVVG) (SEQ ID NO: 2), relative to a corresponding linear peptide.

In an embodiment, the antibody specifically and/or selectively binds a cyclic compound comprising an epitope peptide sequence described herein comprising at least one alternate conformational feature described herein (e.g. of the epitope in a cyclic compound compared to a linear compound). For example an antibody that binds a particular epitope conformation can be referred to as a conformation specific antibody. Such antibodies can be selected using the methods described herein. The conformation specific antibody can differentially recognize a particular Abeta species or a group of related species (e.g. dimers, trimers, and other oligomeric species) and can have a higher affinity for one species or group of species compared to another (e.g. to either the monomer or fibril species).

In an embodiment, the antibody does not specifically bind monomeric A-beta. In an embodiment, the antibody does not specifically bind A-beta senile plaques, for example in situ in AD brain tissue.

In another embodiment, the antibody does not selectively bind monomeric A-beta compared to native- or synthetic-oligomeric A-beta.

For example, the antibody may specifically bind a cyclic compound comprises a residue selected from G and V, wherein at least one dihedral angle is at least 30 degrees, at least 40 degrees, at least 50 degrees, at least 60 degrees, at least 70 degrees, at least 80 degrees, at least 90 degrees, at least 100 degrees, at least 110 degrees, at least 120 degrees, at least 130 degrees, at least 140 degrees at least 150 degrees different in the cyclic compound, than the corresponding dihedral angle in the context of the linear compound.

In an embodiment, the antibody selectively binds A-beta peptide in a cyclic compound, the A-beta comprising GGVV (SEQ ID NO: 1) or a part thereof, optionally in the context of cyclo (CGGGVVG) (SEQ ID NO: 2) relative to a linear peptide comprising GGVV (SEQ ID NO: 1), optionally in the context of linear CGGGVVG (SEQ ID NO: 2), such as a corresponding sequence. For example, in an embodiment the antibody selectively binds GGVV (SEQ ID NO: 1) in a cyclic conformation and has at least 2 fold, at least 3 fold, at least 5 fold, at least 10 fold at least 20 fold, at least 30 fold, at least 40 fold, at least 50 fold, at least 100 fold, at least 500 fold, at least 1000 fold more selective greater selectivity (e.g. binding affinity) for GGVV (SEQ ID NO: 1) in the cyclic conformation compared to GGVV (SEQ ID NO: 1) in a linear peptide, for example as measured by ELISA or surface plasmon resonance, or optionally using a method described herein.

In an embodiment, the cyclic compound is cyclo(CGGGVVG) (SEQ ID NO: 2).

In an embodiment, the antibody selectively binds A-beta peptide in a cyclic compound and/or A-beta. In an embodiment, the selectivity is at least 2 fold, at least 3 fold, at least 5 fold, at least 10 fold, at least 20 fold, at least 30 fold, at least 40 fold, at least 50 fold, at least 100 fold, at least 500 fold, at least 1000 fold more selective for the A-beta peptide in the cyclic compound and/or A-beta oligomer over a species of A-beta selected from A-beta monomer and/or A-beta fibril and/or linear GGVV (SEQ ID NO: 1), optionally linear CGGGVVG (SEQ ID NO: 2).

In an embodiment, the Abeta oligomer comprises Abeta 1-42 subunits.

In an embodiment, the antibody lacks A-beta fibril plaque (also referred to as senile plaque) staining. Absence of plaque staining can be assessed by comparing to a positive control such as A-beta-specific antibodies 6E10 and 4G8 (Biolegend, San Diego, Calif.), or 2C8 (Enzo Life Sciences Inc., Farmingdale, N.Y.), or any other antibody reactive to fibrillar forms of A-beta, and an isotype control. An antibody described herein lacks or has negligible A-beta fibril plaque staining if the antibody does not show typical plaque morphology staining and the level of staining is comparable to or no more than 2 fold the level seen with an IgG negative isotype control. The scale can for example set the level of staining with isotype control at 1 and with 6E10 at 10. An antibody lacks A-beta fibril plaque staining if the level of staining on such a scale is 2 or less. In embodiment, the antibody shows minimal A-beta fibril plaque staining, for example on the foregoing scale, levels scored at less about or less than 3.

In an embodiment, the antibody is a monoclonal antibody.

To produce monoclonal antibodies, antibody producing cells (lymphocytes) can be harvested from a subject immunized with an immunogen described herein, and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with the desired epitopes and the monoclonal antibodies can be isolated.

Specific antibodies, or antibody fragments, reactive against particular antigens or molecules, may also be generated by screening expression libraries encoding immunoglobulin genes, or portions thereof, expressed in bacteria with cell surface components. For example, complete Fab fragments, VH regions and FV regions can be expressed in bacteria using phage expression libraries (see for example Ward et al., Nature 41:544-546 (1989); Huse et al., Science 246:1275-1281 (1989); and McCafferty et al., Nature 348:552-554 (1990).

In an embodiment, the antibody is a humanized antibody.

The humanization of antibodies from non-human species has been well described in the literature. See for example EP-B1 0 239400 and Carter & Merchant 1997 (Curr Opin Biotechnol 8, 449-454, 1997 incorporated by reference in their entirety herein). Humanized antibodies are also readily obtained commercially (e.g. Scotgen Limited, 2 Holly Road, Twickenham, Middlesex, Great Britain.).

Humanized forms of rodent antibodies are readily generated by CDR grafting (Riechmann et al. Nature, 332:323-327, 1988). In this approach the six CDR loops comprising the antigen binding site of the rodent monoclonal antibody are linked to corresponding human framework regions. CDR grafting often yields antibodies with reduced affinity as the amino acids of the framework regions may influence antigen recognition (Foote & Winter. J Mol Biol, 224: 487-499, 1992). To maintain the affinity of the antibody, it is often necessary to replace certain framework residues by site directed mutagenesis or other recombinant techniques and may be aided by computer modeling of the antigen binding site (Co et al. J Immunol, 152: 2968-2976, 1994).

Humanized forms of antibodies are optionally obtained by resurfacing (Pedersen et al. J Mol Biol, 235: 959-973, 1994). In this approach only the surface residues of a rodent antibody are humanized.

Human antibodies specific to a particular antigen may be identified by a phage display strategy (Jespers et al. Bio/Technology, 12: 899-903, 1994). In one approach, the heavy chain of a rodent antibody directed against a specific antigen is cloned and paired with a repertoire of human light chains for display as Fab fragments on filamentous phage. The phage is selected by binding to antigen. The selected human light chain is subsequently paired with a repertoire of human heavy chains for display on phage, and the phage is again selected by binding to antigen. The result is a human antibody Fab fragment specific to a particular antigen. In another approach, libraries of phage are produced where members display different human antibody fragments (Fab or Fv) on their outer surfaces (Dower et al., WO 91/17271 and McCafferty et al., WO 92/01047). Phage displaying antibodies with a desired specificity are selected by affinity enrichment to a specific antigen. The human Fab or Fv fragment identified from either approach may be recloned for expression as a human antibody in mammalian cells.

Human antibodies are optionally obtained from transgenic animals (U.S. Pat. Nos. 6,150,584; 6,114,598; and 5,770,429). In this approach the heavy chain joining region (JH) gene in a chimeric or germ-line mutant mouse is deleted. Human germ-line immunoglobulin gene array is subsequently transferred to such mutant mice. The resulting transgenic mouse is then capable of generating a full repertoire of human antibodies upon antigen challenge.

Humanized antibodies are typically produced as antigen binding fragments such as Fab, Fab′ F(ab′)2, Fd, Fv and single domain antibody fragments, or as single chain antibodies in which the heavy and light chains are linked by a spacer. Also, the human or humanized antibodies may exist in monomeric or polymeric form. The humanized antibody optionally comprises one non-human chain and one humanized chain (i.e. one humanized heavy or light chain).

Antibodies, including humanized or human antibodies, are selected from any class of immunoglobulins including: IgM, IgG, IgD, IgA or IgE; and any isotype, including: IgG1, IgG2, IgG3 and IgG4. A chimeric, humanized or human antibody may include sequences from one or more than one isotype or class.

Additionally, antibodies specific for the epitopes described herein are readily isolated by screening antibody phage display libraries. For example, an antibody phage library is optionally screened by using a disease specific epitope of the current invention to identify antibody fragments specific for the disease specific epitope. Antibody fragments identified are optionally used to produce a variety of recombinant antibodies that are useful with different embodiments described herein. Antibody phage display libraries are commercially available, for example, through Xoma (Berkeley, Calif.) Methods for screening antibody phage libraries are well known in the art.

A further aspect is antibody and/or binding fragment thereof comprising a light chain variable region and a heavy chain variable region, the heavy chain variable region comprising complementarity determining regions CDR-H1, CDR-H2 and CDR-H3, the light chain variable region comprising complementarity determining region CDR-L1, CDR-L2 and CDR-L3 and with the amino acid sequences of said CDRs comprising the sequences set forth below:

| | | |
|---|---|---|
| CDR-H1 | GFTFSNYW | SEQ ID NO: 17 |
| CDR-H2 | IRLKSYNYAT | SEQ ID NO: 18 |
| CDR-H3 | LRWIDY | SEQ ID NO: 19 |
| CDR-L1 | QDINSY | SEQ ID NO: 20 |
| CDR-L2 | RAN | SEQ ID NO: 21 |
| CDR-L3 | PQYDEFPYT | SEQ ID NO: 22 |

In an embodiment, the antibody is a monoclonal antibody. In an embodiment, the antibody is a chimeric antibody such as a humanized antibody comprising the CDR sequences as recited in Table 12.

Another aspect includes an antibody that competes for binding to human A-beta with an antibody comprising the CDR sequences as recited in Table 12.

Also provided in another embodiment, is an antibody comprising the CDRs in Table 12 and a light chain variable region and a heavy chain variable region, optionally in the context of a single chain antibody.

In yet another aspect, the antibody comprises a heavy chain variable region comprises: i) an amino acid sequence as set forth in SEQ ID NO: 24; ii) an amino acid sequence with at least 50%, at least 60%, at least 70%, at least 80% or at least 90% sequence identity to SEQ ID NO: 24, wherein the CDR sequences are as set forth in SEQ ID NO: 17, 18 and 19, or iii) a conservatively substituted amino acid sequence i). In another aspect the antibody comprises a light chain variable region comprising i) an amino acid sequence as set forth in SEQ ID NO: 26, ii) an amino acid sequence with at least 50%, at least 60%, at least 70%, at least 80% or at least 90% sequence identity to SEQ ID NO: 26, wherein the CDR sequences are as set forth in SEQ ID NO: 20, 21 and 22, or iii) a conservatively substituted amino acid sequence of i). In another embodiment, the heavy chain variable region amino acid sequence is encoded by a nucleotide sequence as set out in SEQ ID NO: 23 or a codon degenerate optimized version thereof. In another embodiment, the antibody comprises a light chain variable region amino acid sequence encoded by a nucleotide sequence as set out in SEQ ID NO: 25 or a codon degenerate or optimized version thereof. In an embodiment, the heavy chain variable region comprises an amino acid sequence as set forth in SEQ ID NO: 24

Another aspect is an antibody that specifically binds a same epitope as the antibody with CDR sequences as recited in Table 12.

Competition between antibodies can be determined for example using an assay in which an antibody under test is assessed for its ability to inhibit specific binding of a reference antibody to the common antigen. A test antibody competes with a reference antibody if an excess of a test antibody (e.g., at least a 2 fold, 5, fold, 10 fold or 20 fold) inhibits binding of the reference antibody by at least 50%, at least 75%, at least 80%, at least 90% or at least 95% as measured in a competitive binding assay.

A further aspect is an antibody conjugated to a therapeutic, detectable label or cytotoxic agent. In an embodiment, the detectable label is a positron-emitting radionuclide. A positron-emitting radionuclide can be used for example in PET imaging.

A further aspect relates to an antibody complex comprising an antibody described herein and/or a binding fragment thereof and oligomeric A-beta.

A further aspect is an isolated nucleic acid encoding an antibody or part thereof described herein.

Nucleic acids encoding a heavy chain or a light chain are also provided, for example encoding a heavy chain comprising CDR-H1, CDR-H2 and/or CDR-H3 regions described herein or encoding a light chain comprising CDR-L1, CDR-L2 and/or CDR-L3 regions described herein.

The present disclosure also provides variants of the nucleic acid sequences that encode for the antibody and/or binding fragment thereof disclosed herein. For example, the variants include nucleotide sequences that hybridize to the nucleic acid sequences encoding the antibody and/or binding fragment thereof disclosed herein under at least moderately stringent hybridization conditions or codon degenerate or optimized sequences In another embodiment, the variant nucleic acid sequences have at least 50%, at least 60%, at least 70%, most preferably at least 80%, even more preferably at least 90% and even most preferably at least 95% sequence identity to nucleic acid sequences encoding SEQ ID NOs: 24 and 26.

In an embodiment, the nucleic acid is an isolated nucleic acid.

Another aspect is an expression cassette or a vector comprising the nucleic acid herein disclosed. In an embodiment, the vector is an isolated vector.

The vector can be any vector, including vectors suitable for producing an antibody and/or binding fragment thereof or expressing an epitope peptide sequence described herein.

The nucleic acid molecules may be incorporated in a known manner into an appropriate expression vector which ensures expression of the protein. Possible expression vectors include but are not limited to cosmids, plasmids, or modified viruses (e.g. replication defective retroviruses, adenoviruses and adeno-associated viruses). The vector should be compatible with the host cell used. The expression vectors are “suitable for transformation of a host cell”, which means that the expression vectors contain a nucleic acid molecule encoding the peptides corresponding to epitopes or antibodies described herein.

In an embodiment, the vector is suitable for expressing for example single chain antibodies by gene therapy. The vector can be adapted for specific expression in neural tissue, for example using neural specific promoters and the like. In an embodiment, the vector comprises an IRES and allows for expression of a light chain variable region and a heavy chain variable region. Such vectors can be used to deliver antibody in vivo.

Suitable regulatory sequences may be derived from a variety of sources, including bacterial, fungal, viral, mammalian, or insect genes.

Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

In an embodiment, the regulatory sequences direct or increase expression in neural tissue and/or cells.

In an embodiment, the vector is a viral vector.

The recombinant expression vectors may also contain a marker gene which facilitates the selection of host cells transformed, infected or transfected with a vector for expressing an antibody or epitope peptide described herein.

The recombinant expression vectors may also contain genes which encode a fusion moiety (i.e. a “fusion protein”) which provides increased expression or stability of the recombinant peptide; increased solubility of the recombinant peptide; and aid in the purification of the target recombinant peptide by acting as a ligand in affinity purification, including for example tags and labels described herein. Further, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Systems for the transfer of genes for example into neurons and neural tissues both in vitro and in vivo include vectors based on viruses, most notably Herpes Simplex Virus, Adenovirus, Adeno-associated virus (AAV) and retroviruses including lentiviruses. Alternative approaches for gene delivery include the use of naked, plasmid DNA as well as liposome-DNA complexes. Another approach is the use of AAV plasmids in which the DNA is polycation-condensed and lipid entrapped and introduced into the brain by intracerebral gene delivery (Leone et al. US Application No. 2002076394).

Accordingly in another aspect, the compounds, immunogens, nucleic acids, vectors and antibodies described herein may be formulated in vesicles such as liposomes, nanoparticles, and viral protein particles, for example for delivery of antibodies, compounds, immunogens and nucleic acids described herein. In particular synthetic polymer vesicles, including polymersomes, can be used to administer antibodies.

Also provided in another aspect is a cell, optionally an isolated and/or recombinant cell, expressing an antibody described herein or comprising an expression cassette or vector herein disclosed.

The recombinant cell can be generated using any cell suitable for producing a polypeptide, for example suitable for producing an antibody and/or binding fragment thereof. For example to introduce a nucleic acid (e.g. a vector) into a cell, the cell may be transfected, transformed or infected, depending upon the vector employed.

Suitable host cells include a wide variety of prokaryotic and eukaryotic host cells. For example, the peptides and antibodies described herein may be expressed in bacterial cells such as E. coli, insect cells (using baculovirus), yeast cells or mammalian cells.

In an embodiment, the cell is a eukaryotic cell selected from a yeast, plant, worm, insect, avian, fish, reptile and mammalian cell.

In another embodiment, the mammalian cell is a myeloma cell, a spleen cell, or a hybridoma cell.

In an embodiment, the cell is a neural cell.

Yeast and fungi host cells suitable for expressing an antibody or peptide include, but are not limited to Saccharomyces cerevisiae, Schizosaccharomyces pombe, the genera Pichia or Kluyveromyces and various species of the genus Aspergillus. Examples of vectors for expression in yeast S. cerivisiae include pYepSec1, pMFa, pJRY88, and pYES2 (Invitrogen Corporation, San Diego, Calif.). Protocols for the transformation of yeast and fungi are well known to those of ordinary skill in the art.

Mammalian cells that may be suitable include, among others: COS (e.g., ATCC No. CRL 1650 or 1651), BHK (e.g. ATCC No. CRL 6281), CHO (ATCC No. CCL 61), HeLa (e.g., ATCC No. CCL 2), 293 (ATCC No. 1573) and NS-1 cells. Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include pCDM8 and pMT2PC.

A further aspect is a hybridoma producing an antibody specific for an epitope described herein.

### III. Compositions

A further aspect is a composition comprising a compound, immunogen, nucleic acid, vector or antibody described herein.

In an embodiment, the composition comprises a diluent.

Suitable diluents for nucleic acids include but are not limited to water, saline solutions and ethanol.

Suitable diluents for polypeptides, including antibodies or fragments thereof and/or cells include but are not limited to saline solutions, pH buffered solutions and glycerol solutions or other solutions suitable for freezing polypeptides and/or cells.

In an embodiment, the composition is a pharmaceutical composition comprising any of the peptides, immunogens, antibodies, nucleic acids or vectors disclosed herein, and optionally comprising a pharmaceutically acceptable carrier.

The compositions described herein can be prepared by per se known methods for the preparation of pharmaceutically acceptable compositions that can be administered to subjects, optionally as a vaccine, such that an effective quantity of the active substance is combined in a mixture with a pharmaceutically acceptable vehicle.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the patient.

Pharmaceutical compositions may comprise a pharmaceutically acceptable carrier. Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesylphosphotidyl-ethanolamine (DOPE), and liposomes. Such compositions should contain a therapeutically effective amount of the compound, together with a suitable amount of carrier so as to provide the form for direct administration to the patient.

The composition may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylarnino ethanol, histidine, procaine, etc.

In an embodiment comprising a compound or immunogen described herein, the composition comprises an adjuvant.

Adjuvants that can be used for example, include Intrinsic adjuvants (such as lipopolysaccharides) normally are the components of killed or attenuated bacteria used as vaccines. Extrinsic adjuvants are immunomodulators which are typically non-covalently linked to antigens and are formulated to enhance the host immune responses. Aluminum hydroxide, aluminum sulfate and aluminum phosphate (collectively commonly referred to as alum) are routinely used as adjuvants. A wide range of extrinsic adjuvants can provoke potent immune responses to immunogens. These include saponins such as Stimulons (QS21, Aquila, Worcester, Mass.) or particles generated therefrom such as ISCOMs and (immunostimulating complexes) and ISCOMATRIX, complexed to membrane protein antigens (immune stimulating complexes), pluronic polymers with mineral oil, killed mycobacteria and mineral oil, Freund's complete adjuvant, bacterial products such as muramyl dipeptide (MDP) and lipopolysaccharide (LPS), as well as lipid A, and liposomes.

In an embodiment, the adjuvant is aluminum hydroxide. In another embodiment, the adjuvant is aluminum phosphate. Oil in water emulsions include squalene; peanut oil; MF59 (WO 90/14387); SAF (Syntex Laboratories, Palo Alto, Calif.); and Ribi™ (Ribi Immunochem, Hamilton, Mont.). Oil in water emulsions may be used with immunostimulating agents such as muramyl peptides (for example, N-acetylmuramyl-L-threonyl-D-isoglutamine (thr-MDP), -acetyl-normuramyl-L-alanyl-D-isoglutamine (nor-MDP), N-acetylmuramyl-L-alanyl-D-isoglutamyl-L-alanine-2-(1′-2′dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) theramide (TM)), or other bacterial cell wall components.

The adjuvant may be administered with an immuogen as a single composition. Alternatively, an adjuvant may be administered before, concurrent and/or after administration of the immunogen.

Commonly, adjuvants are used as a 0.05 to 1.0 percent solution in phosphate-buffered saline. Adjuvants enhance the immunogenicity of an immunogen but are not necessarily immunogenic themselves. Adjuvants may act by retaining the immunogen locally near the site of administration to produce a depot effect facilitating a slow, sustained release of immunogen to cells of the immune system. Adjuvants can also attract cells of the immune system to an immunogen depot and stimulate such cells to elicit immune responses. As such, embodiments encompass compositions further comprising adjuvants.

Adjuvants for parenteral immunization include aluminum compounds (such as aluminum hydroxide, aluminum phosphate, and aluminum hydroxy phosphate). The antigen can be precipitated with, or adsorbed onto, the aluminum compound according to standard protocols. Other adjuvants such as RIBI (ImmunoChem, Hamilton, Mont.) can also be used in parenteral administration.

Adjuvants for mucosal immunization include bacterial toxins (e.g., the cholera toxin (CT), the E. coli heat-labile toxin (LT), the Clostridium difficile toxin A and the pertussis toxin (PT), or combinations, subunits, toxoids, or mutants thereof). For example, a purified preparation of native cholera toxin subunit B (CTB) can be of use. Fragments, homologs, derivatives, and fusion to any of these toxins are also suitable, provided that they retain adjuvant activity. Preferably, a mutant having reduced toxicity is used. Suitable mutants have been described (e.g., in WO 95/17211 (Arg-7-Lys CT mutant), WO 96/6627 (Arg-192-Gly LT mutant), and WO 95/34323 (Arg-9-Lys and Glu-129-Gly PT mutant)). Additional LT mutants that can be used in the methods and compositions include, for example Ser-63-Lys, Ala-69-Gly, Glu-110-Asp, and Glu-112-Asp mutants. Other adjuvants (such as a bacterial monophosphoryl lipid A (MPLA) of various sources (e.g., E. coli, Salmonella minnesota, Salmonella typhimurium, or Shigella flexneri, saponins, or polylactide glycolide (PLGA) microspheres) can also be used in mucosal administration.

Other adjuvants include cytokines such as interleukins for example IL-1, IL-2 and IL-12, chemokines, for example CXCL10 and CCL5, macrophage stimulating factor, and/or tumor necrosis factor. Other adjuvants that may be used include CpG oligonucleotides (Davis. Curr Top Microbiol Immunol., 247:171-183, 2000).

Oil in water emulsions include squalene; peanut oil; MF59 (WO 90/14387); SAF (Syntex Laboratories, Palo Alto, Calif.); and Ribi™ (Ribi Immunochem, Hamilton, Mont.). Oil in water emulsions may be used with immunostimulating agents such as muramyl peptides (for example, N-acetylmuramyl-L-threonyl-D-isoglutamine (thr-MDP), -acetyl-normuramyl-L-alanyl-D-isoglutamine (nor-MDP), N-acetylmuramyl-L-alanyl-D-isoglutamyl-L-alanine-2-(1′-2′dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) theramide (TM)), or other bacterial cell wall components.

Adjuvants useful for both mucosal and parenteral immunization include polyphosphazene (for example, WO 95/2415), DC-chol (3 b-(N—(N′,N′-dimethyl aminomethane)-carbamoyl) cholesterol (for example, U.S. Pat. No. 5,283,185 and WO 96/14831) and QS-21 (for example, WO 88/9336).

An adjuvant may be coupled to an immunogen for administration. For example, a lipid such as palmitic acid, may be coupled directly to one or more peptides such that the change in conformation of the peptides comprising the immunogen does not affect the nature of the immune response to the immunogen.

The adjuvant may be administered with an immuogen as a single composition. Further, an adjuvant may be administered before, concurrent or after administration of the immunogen.

In an embodiment, the composition comprises an antibody described herein. In another embodiment, the composition comprises an antibody described herein and a diluent. In an embodiment, the composition is a sterile composition.

A further aspect includes an antibody complex comprising an antibody described herein and A-beta, optionally A-beta oligomer. The complex may be in solution or comprised in a tissue, optionally in vitro.

### IV. Kits

A further aspect relates to a kit comprising i) an antibody and/or binding fragment thereof, ii) a nucleic acid, iii) peptide or immunogen, iv) composition or v) recombinant cell described herein, comprised in a vial such as a sterile vial or other housing and optionally a reference agent and/or instructions for use thereof.

In an embodiment, the kit further comprises one or more of a collection vial, standard buffer and detection reagent.

### V. Methods

Included are methods for making and using the compounds, immunogens and antibodies described herein.

In particular, provided are methods of making an antibody specific and/or selective for a conformational epitope of GGVV (SEQ ID NO: 1) or related epitope comprising administering to a subject, optionally a non-human subject, a conformationally restricted compound comprising an epitope sequence described herein, optionally cyclic compound comprising GGVV (SEQ ID NO: 1) or related epitope, and isolating antibody producing cells or antibodies that specifically or selectively bind the cyclic compound and optionally i) specifically or selectively bind synthetic and/or native oligomers and/or that have no or negligible senile plaque binding in situ tissue samples or no or negligible binding to a linear compound, optionally a corresponding linear peptide. The cyclic compound can for example comprise any of the “epitopes” described herein containing cyclic compounds described herein.

In an embodiment, the method is for making a monoclonal antibody using for example a method as described herein.

In an embodiment, the method is for making a humanized antibody using for example a method described herein.

Antibodies produced using a cyclic compound are selected as described herein and in the Examples. In an embodiment, the method comprises isolating antibodies that specifically or selectively bind cyclic peptide over linear peptide, are specific for the epitope sequence, specifically bind oligomer and/or lack or negligibly bind plaque in situ and/or corresponding linear peptide, optionally using a method described herein.

A further aspect provides a method of detecting whether a biological sample comprises A-beta the method comprising contacting the biological sample with an antibody described herein and detecting the presence of any antibody complex. In an embodiment, the method is for detecting whether a biological sample comprises A-beta wherein at least one of the residues G or V is in an alternate conformation than occupied by G and/or V in a non-oligomeric conformation. In an embodiment the method is for detecting whether the biologic sample comprises oligomeric A-beta.

In an embodiment, the method comprises:
a. contacting the biologic sample with an antibody described herein that is specific and/or selective for A-beta oligomer herein under conditions permissive to produce an antibody:A-beta oligomer complex; and
b. detecting the presence of any complex;
   wherein the presence of detectable complex is indicative that the sample may contain A-beta oligomer.

In an embodiment, the level of complex formed is compared to a test antibody such as a suitable Ig control or irrelevant antibody.

In an embodiment, the detection is quantitated and the amount of complex produced is measured. The measurement can for example be relative to a standard.

In an embodiment, the measured amount is compared to a control.

In another embodiment, the method comprises:
(a) contacting a biological sample of said subject with an antibody described herein, under conditions permissive to produce an antibody-antigen complex;
(b) measuring the amount of the antibody-antigen complex in the test sample; and
(c) comparing the amount of antibody-antigen complex in the test sample to a control;
   wherein detecting antibody-antigen complex in the biological sample as compared to the control indicates that the sample comprises A-beta.

The control can be a sample control (e.g. from a subject without AD, or from a subject with a particular form of AD, mild, moderate or advanced), or be a previous sample from the same subject for monitoring changes in A-beta oligomer levels in the subject.

In an embodiment, an antibody described herein is used.

In an embodiment, the antibody specifically and/or selectively recognizes a conformation of A-beta comprising a GGVV (SEQ ID NO: 1) or related conformational epitope, and detecting the antibody antigen complex in the biological sample is indicative that sample comprises A-beta oligomer.

In an embodiment, the sample is a biological sample. In an embodiment, the sample comprises brain tissue or an extract thereof and/or CSF. In an embodiment, the sample comprises whole blood, plasma or serum. In an embodiment, the sample is obtained from a human subject. In an embodiment, the subject is suspected of, at a risk of or has AD.

A number of methods can be used to detect an A-beta:antibody complex and thereby determine if A-beta comprising a GGVV (SEQ ID NO: 1) or related conformational epitope and/or A-beta oligomers is present in the biological sample using the antibodies described herein, including immunoassays such as flow cytometry, Western blots, ELISA, and immunoprecipitation followed by SDS-PAGE immunocytochemistry.

As described in the Examples surface plasmon resonance technology can be used to assess conformation specific binding. If the antibody is labeled or a detectably labeled secondary antibody specific for the complex antibody is used, the label can be detected. Commonly used reagents include fluorescent emitting and HRP labeled antibodies. In quantitative methods, the amount of signal produced can be measured by comparison to a standard or control. The measurement can also be relative.

A further aspect includes a method of measuring a level of or imaging A-beta in a subject or tissue, optionally where the A-beta to be measured or imaged is oligomeric A-beta. In an embodiment, the method comprises administering to a subject at risk or suspected of having or having AD, an antibody conjugated to a detectable label; and detecting the label, optionally quantitatively detecting the label. The label in an embodiment is a positron emitting radionuclide which can for example be used in PET imaging.

A further aspect includes a method of inducing an immune response in a subject, comprising administering to the subject a compound described herein, optionally a cyclic compound comprising GGVV (SEQ ID NO: 1) or a related epitope peptide sequence, an immunogen and/or composition comprising said compound or said immunogen; and optionally isolating cells and/or antibodies that specifically and/or selectively bind the A-beta peptide in the compound or immunogen administered. In an embodiment, the composition is a pharmaceutical composition comprising the compound or immunogen in admixture with a pharmaceutically acceptable, diluent or carrier.

In an embodiment, the subject is a non-human subject such as a rodent. Antibody producing cells generated are used in an embodiment to produce a hybridoma cell line.

In an embodiment, the immunogen administered comprises a compound of FIG. 7C.

It is demonstrated herein that antibodies raised against cyclo(CGGGVVG) (SEQ ID NO: 2), can specifically and/or selectively bind A-beta oligomers and lack A-beta plaque staining. Oligomeric A-beta species are believed to be the toxic propagating species in AD. Further as shown in FIG. 19, antibody raised using cyclo(CGGGVVG) (SEQ ID NO: 2) and specific for oligomers, inhibited A-beta aggregation and A-beta oligomer propagation. Accordingly, also provided are methods of inhibiting A-beta oligomer propagation, the method comprising contacting a cell or tissue expressing A-beta with or administering to a subject in need thereof an effective amount of an A-beta oligomer specific and/or selective antibody described herein to inhibit A-beta aggregation and/or oligomer propagation. In vitro the assay can be monitored as described in Example 10.

The antibodies may also be useful for treating AD and/or other A-beta amyloid related diseases. For example, variants of Lewy body dementia and in inclusion body myositis (a muscle disease) exhibit similar plaques as AD and A-beta can also form aggregates implicated in cerebral amyloid angiopathy. As mentioned, antibodies raised to cyclo(CGGGVVG) (SEQ ID NO: 2) bind oligomeric A-beta which is believed to be a toxigenic species of A-beta in AD and inhibit formation of toxigenic A-beta oligomers.

Accordingly a further aspect is a method of treating AD and/or other A-beta amyloid related diseases, the method comprising administering to a subject in need thereof i) an effective amount of an antibody described herein, optionally an A-beta oligomer specific and/or selective antibody or a pharmaceutical composition comprising said antibody; or 2) administering an isolated cyclic compound comprising GGVV (SEQ ID NO: 1) or a related epitope sequence or immunogen or pharmaceutical composition comprising said cyclic compound, to a subject in need thereof.

In an embodiment, a biological sample from the subject to be treated is assessed for the presence or levels of A-beta using an antibody described herein. In an embodiment, a subject with detectable A-beta levels (e.g. A-beta antibody complexes measured in vitro or measured by imaging) is treated with the antibody.

The antibody and immunogens can for example be comprised in a pharmaceutical composition as described herein, and formulated for example in vesicles for improving delivery.

One or more antibodies targeting described herein for example one or more antibodies targeting GGVV (SEQ ID NO: 1) and/or related antibodies presented in a cyclic compound can be administered in combination. In addition the antibodies disclosed herein can be administered with one or more other treatments such as a beta-secretase inhibitor or a cholinesterase inhibitor.

In an embodiment, the antibody is a conformation specific/selective antibody, optionally that specifically or selectively binds A-beta oligomer.

Also provided are uses of the compositions, antibodies, isolated peptides, immunogens and nucleic acids for treating AD.

The compositions, compounds, antibodies, isolated peptides, immunogens and nucleic acids, vectors etc. described herein can be administered for example, by parenteral, intravenous, subcutaneous, intramuscular, intracranial, intraventricular, intrathecal, intraorbital, ophthalmic, intraspinal, intracisternal, intraperitoneal, intranasal, aerosol or oral administration.

In certain embodiments, the pharmaceutical composition is administered systemically.

In other embodiments, the pharmaceutical composition is administered directly to the brain or other portion of the CNS. For example such methods include the use of an implantable catheter and a pump, which would serve to discharge a pre-determined dose through the catheter to the infusion site. A person skilled in the art would further recognize that the catheter may be implanted by surgical techniques that permit visualization of the catheter so as to position the catheter adjacent to the desired site of administration or infusion in the brain. Such techniques are described in Elsberry et al. U.S. Pat. No. 5,814,014 “Techniques of Treating Neurodegenerative Disorders by Brain Infusion”, which is herein incorporated by reference. Also contemplated are methods such as those described in US patent application 20060129126 (Kaplitt and During “Infusion device and method for infusing material into the brain of a patient”. Devices for delivering drugs to the brain and other parts of the CNS are commercially available (e.g. SynchroMed® EL Infusion System; Medtronic, Minneapolis, Minn.)

In another embodiment, the pharmaceutical composition is administered to the brain using methods such as modifying the compounds to be administered to allow receptor-mediated transport across the blood brain barrier.

Other embodiments contemplate the co-administration of the compositions, compounds, antibodies, isolated peptides, immunogens and nucleic acids described herein with biologically active molecules known to facilitate the transport across the blood brain barrier.

Also contemplated in certain embodiments, are methods for administering the compositions, compounds, antibodies, isolated peptides, immunogens and nucleic acids described herein across the blood brain barrier such as those directed at transiently increasing the permeability of the blood brain barrier as described in U.S. Pat. No. 7,012,061 “Method for increasing the permeability of the blood brain barrier”, herein incorporated by reference.

A person skilled in the art will recognize the variety of suitable methods for administering the compositions, compounds, antibodies, isolated peptides, immunogens and nucleic acids described herein directly to the brain or across the blood brain barrier and be able to modify these methods in order to safely administer the products described herein.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Collective Coordinates Predictions

A method for predicting misfolded epitopes is provided by a method referred to as “Collective Coordinates biasing” which is described in U.S. Patent Application Ser. No. 62/253,044, SYSTEMS AND METHODS FOR PREDICTING MISFOLDED PROTEIN EPITOPES BY COLLECTIVE COORDINATE BIASING filed Nov. 9, 2015, and is incorporated herein by reference. As described therein, the method uses molecular-dynamics-based simulations which impose a global coordinate bias on a protein (or peptide-aggregate) to force the protein (or peptide-aggregate) to misfold and then predict the most likely unfolded regions of the partially unstructured protein (or peptide aggregate). Biasing simulations were performed and the solvent accessible surface area (SASA) corresponding to each residue index (compared to that of the initial structure of the protein under consideration). SASA represents a surface area that is accessible to H₂O. A positive change in SASA (compared to that of the initial structure of the protein under consideration) may be considered to be indicative of unfolding in the region of the associated residue index. The method was applied to a single-chain, and three A-beta strains, each with its own morphology: a three-fold symmetric structure of Aβ-40 peptides (or monomers) (PDB entry 2M4J), a two-fold symmetric structure of Aβ-40 monomers (PDB entry 2LMN), and a single-chain, parallel in-register (e.g. a repeated beta sheet where the residues from one chain interact with the same residues from the neighboring chains) structure of Aβ-42 monomers (PDB entry 2MXU).

Simulations were performed for each initial structure using the collective coordinates method as described in U.S. Patent Application Ser. No. 62/253,044, and the CHARMM force-field parameters described in: K. Vanommeslaeghe, E. Hatcher, C. Acharya, S. Kundu, S. Zhong, J. Shim, E. Darian, O. Guvench, P. Lopes, I. Vorobyov, and A. D. Mackerell. Charmm general force field: A force field for drug-like molecules compatible with the charmm all-atom additive biological force fields. Journal of Computational Chemistry, 31(4):671-690, 2010; and P. Bjelkmar, P. Larsson, M. A. Cuendet, B. Hess, and E. Lindahl. Implementation of the CHARMM force field in GROMACS: analysis of protein stability effects from correlation maps, virtual interaction sites, and water models. J. Chem. Theo. Comp., 6:459-466, 2010, both of which are hereby incorporated herein by reference, with TIP3P water.

Epitopes predicted using this method are described in Example 2.

### Gō Model Method for Predicting A-Beta Oligomer Specific Epitopes

A second epitope prediction model is based on the free energy landscape of partial protein unfolding from the native state. The native state is taken to be an experimentally-derived fibril structure. When the protein is partially unfolded from the native state by a given amount of primary sequence, epitope candidates are contiguous sequence segments that cost the least free energy to disorder. The free energy of a given protein conformation arises from several contributions, including conformational entropy and solvation of polar functional groups that favor the unfolded state, as well as the loss of electrostatic and van der Waals intra-protein interactions that enthalpically stabilize the native state.

### A. Gō-Like Model of Protein Partially Unfolding Landscape

An approximate model to account for the free energetic changes that take place during unfolding assigns a fixed energy to all contacts in the native state, where a contact is defined as a pair of heavy (non-hydrogen) atoms within a fixed cut-off distance r_{cutoff}. Gō-like models have been successfully implemented in previous studies of protein folding. The Gō-like model isolates the effects arising from the topology of native protein interactions, and in practice the unfolding free energy landscape can be readily calculated from a single native state structure.

The total free energy cost of unfolding a segment depends on the number of interactions to be disrupted, together with the conformational entropy term of the unfolded region.

In the following equations, lower case variables refer to atoms, while upper case variables refer to residues. Let T be the set of all residues in the protein, U be the set of residues unfolded in the protein, and F be the subset of residues folded in the protein (thus T=U ∪ F). The unfolding mechanism at high degrees of nativeness consists of multiple contiguous strands of disordered residues. Here the approximation of a single contiguous unfolded strand was adopted, and the free energy cost to disorderthis contiguous strand was calculated.

The total free energy change ΔF_{Gō}(U) for unfolding the set of residues U is

ΔF_{Gō}(U)=ΔE_{Gō}(U)−TΔS_{Gō}(U) (1)

The unfolding enthalpy function ΔE_{Gō}(U) is given by the number of interactions disrupted by unfolding of the set of U residues:

$$Δ {E}_{G \overline{o}} ( ) = a {\sum }_{Atoms i ∈ , j ∈}^{i > j} ⊖ ( {r}_{cutoff} - \vert {r}_{i} - {r}_{j} \vert ) \qquad ( 2 )$$

In Equation 2, the sum on i, j is over all unique pairs of heavy atoms that have either one or both atoms in the unfolded region, rᵢ and rⱼ are the coordinates of atoms i and j, r_{cutoff} (taken to be 4.8 Å) is the interaction distance cut-off. Θ(x) is the Heaviside function defined by Θ(x)=1 if x is positive and 0 otherwise. The energy per contact a may be chosen to recapitulate the overall experimental stability ΔF_{Exp}(U)|U=T on completely unfolding the protein at room temperature:

$$a = \frac{Δ {F}_{Exp} ( ) {{\vert}_{=} + T Δ {S}_{G \overline{o}} ( ) \vert}_{=}}{{\sum }_{i , j ∈ }^{i > j} ⊖ ( {r}_{cutoff} - \vert {r}_{i} - {r}_{j} \vert )} \qquad ( 3 )$$

The results do not depend on this value; it merely sets the overall global energy scale in the problem. In the present model, this free energy was taken to be a constant number equal to 4.6 kcal/mol. This value is not a primary concern as it is the relative free energy cost for the different regions of the same protein that is sought to be disordered in the method of epitope prediction.

The calculation of the unfolding entropy term ΔS_{Gō}(U) is discussed in B below.

### B. Entropy Calculation

The number of microstates accessible to the protein in the unfolded state is much greater than the number accessible in the native state, so there is a favorable gain of conformational entropy on unfolding. The total entropy of the unfolding segment U by summing over all the residues K in the unfolded region is calculated

$$Δ {S}_{G \overline{o}} ( ) = {\sum }_{K ∈}^{ } ( Δ {S}_{bb , K} - ( 1 - \frac{{A}_{, K}}{{A}_{, K}} ) Δ {S}_{bu \to ex , K} + Δ {S}_{ex \to sol , K} ) \qquad ( 4 )$$

where ΔS_{bb,K}, ΔS_{bu→ex,K}, ΔS_{ex→sol,K} are the three conformational entropic components of residue K as listed in reference [3]: ΔS_{bb,K} is the backbone entropy change from native state to unfolded state, ΔS_{bu→ex,K} is the entropy change for side-chain from buried inside protein to the surface of the protein, and, and ΔS_{ex→sol,K} is the entropy obtained for the side-chain from the surface to the solution.

A correction is applied to the unfolded state conformational entropies, since in the single sequence approximation the end points of the partially unfolded strand are fixed in their positions in the native structure. This means that there is a loop entropy penalty to be paid for constraining the ends in the partially unfolded structure, which is not present in the fully unfolded state

ΔSᵣₑₜᵤᵣₙ=−k_{B} ln(f_{w}(R|N)Δτ). (5)

Here f_{w}(R|N)Δτ is found by calculating the probability an ideal random walk returns to a box of volume Δτ centered at position R after N steps, without penetrating back into the protein during the walk. For strand lengths shorter than about n≈20 residues, the size of the melted strand is much smaller than the protein diameter and the steric excluded volume of the protein is well treated as an impenetrable plane. The number of polymeric states of the melted strand must be multiplied by the fraction of random walks that travel from an origin on the surface of the protein to a location where the melted polymer re-enters the protein without touching or crossing the impenetrable plane. The above fraction of states can be written in the following form:

$${f}_{w} ( R \vert N ) = \frac{a}{{N}^{5 / 2}} exp ( - \frac{3 {R}^{2}}{2 {NI}^{2}} - \frac{{N}^{2} {V}_{c}}{2 {R}^{3}} ) \qquad ( 6 )$$

where R is the end to end distance between the exit and entrance locations, N is the number of residues of the melted region, and a, l, V_{c} are parameters determined by fitting to unfolded polypeptide simulations. The parameter l is the effective arc length between two C_{α} atoms, and V_{c} is the average excluded volumes for each residue. By fitting the Equation 6 into the simulation results, the values of the parameters a=0.0217, l=4.867, V_{c}=3.291 are obtained. This entropy penalty is general and independent of the sequence.

Disulfide bonds require additional consideration in the loop entropy term since they further restrict the motion of the unfolded segment. When present, the disulfide is treated as an additional node through which the loop must pass, in effect dividing the full loop into two smaller loops both subject to the boundary conditions described above.

C. Epitope Prediction from Free Energy Landscape

Once the free energy landscape of partially unfolding the protein is obtained, a variable energy threshold Eₜₕ is applied, and the segments that contains no fewer than 3 amino acids and with free energy cost below the threshold are predicted as epitope candidates. The prediction is stable with respect to varying the threshold value Eₜₕ.

Epitopes predicted using this method are described in Example 2.

### Example 2

### I. Conformation Specific Epitopes

This disclosure pertains to antibodies that may be selective for oligomeric A-beta peptide and particularly to toxic oligomers of Aβ peptide, a species of misfolded protein whose prion-like propagation and interference with synaptic vesicles are believed to be responsible for the synaptic dysfunction and cognitive decline that occurs in Alzheimer's disease (AD). Aβ is a peptide of length 36-43 amino acids that results from the cleavage of amyloid precursor protein (APP) by gamma secretase. In AD patients, it is present in monomers, fibrils, and in soluble oligomers. Aβ is the main component of the amyloid plaques found in the brains of AD patients.

In monomer form, Aβ exists as an unstructured polypeptide chain. In fibril form, Aβ can aggregate into distinct morphologies, often referred to as strains. Several of these structures have been determined by solid-state NMR, some fibril structures have been obtained from in vitro studies, and others obtained by seeding fibrils using amyloid plaques taken from AD patients.

The oligomer is suggested to be a toxic and propagative species of the peptide, recruiting and converting monomeric Aβ to oligomers, and eventually fibrils.

A prerequisite for the generation of oligomer-specific antibodies is the identification of targets on Aβ peptide that are not present on either the monomer or fibril. These oligomer-specific epitopes would not differ in primary sequence from the corresponding segment in monomer or fibril, however they would be conformationally distinct in the context of the oligomer. That is, they would present a distinct conformation in the oligomer that would not be present in the monomer or fibril.

The structure of the oligomer has not been determined to date, moreover, NMR evidence indicates that the oligomer exists not in a single well-defined structure, but in a conformationally-plastic, malleable structural ensemble with limited regularity. Moreover, the concentration of oligomer species is far below either that of the monomer or fibril (estimates vary but on the order of 1000-fold below or more), making this target elusive.

Antibodies directed either against contiguous strands of primary sequence (e.g., linear sequence), or against fibril structures, may suffer from several problems limiting their efficacy. Antibodies raised to linear peptide regions tend not to be selective for oligomer, and thus bind to monomer as well. Because the concentration of monomer is substantially higher than that of oligomer, such antibody therapeutics may suffer from “target distraction”, primarily binding to monomer and promoting clearance of functional Aβ, rather than selectively targeting and clearing oligomeric species. Antibodies raised to amyloid inclusions bind primarily to fibril, and have resulted in amyloid related imaging abnormalities (ARIA), including signal changes thought to represent vasogenic edema and/or microhemorrhages.

To develop antibodies selective for oligomeric forms of Aβ, a region that may be disrupted in the fibril was identified. Without wishing to be bound to theory, it was hypothesized that disruptions in the context of the fibril may be exposed as well on the surface of the oligomer. On oligomers however, these sequence regions may be exposed in conformations distinct from either that of the monomer and/or that of the fibril. For example, being on the surface, they may be exposed in turn regions that have higher curvature, higher exposed surface area, different dihedral angle distribution and/or overall different conformational geometry as determined by structural alignment than the corresponding quantities exhibit in either the fibril or the monomer (e.g. linear peptide).

Cyclic compounds comprising GGVV (SEQ ID NO: 1) are described herein and shown in FIG. 7 Panel C. The cyclic compounds have been designed to satisfy one or more of the above criteria.

A potential benefit of identifying regions prone to disruption in the fibril is that it may identify regions involved in secondary nucleation processes where fibrils may act as a catalytic substrate to nucleate oligomers from monomers [3]. Regions of fibril with exposed side chains may be more likely to engage in aberrant interactions with nearby monomer, facilitating the accretion of monomers; such accreted monomers would then experience an environment of effectively increased concentration at or near the surface of the fibril, and thus be more likely to form multimeric aggregates including oligomers. Aged or damaged fibril with exposed regions of Aβ may enhance the production of toxic oligomer, and that antibodies directed against these disordered regions on the fibril could be effective in blocking such propagative mechanisms.

### II. Collective Coordinates and Promis Gō Predictions

The epitope GGVV (SEQ ID NO: 1) emerges as a predicted epitope from strain 2MXU from the collective coordinates and the Promis Gō approaches described in Example 1. The corresponding figure showing the predicted epitope is in FIG. 1. In Panel A, the graph on the left represents the epitope predictions arising from the partially-disordered fibril, whereas the graph on the right represents epitope predictions arising from the partially-disordered fibril when the two end-capping monomers are positionally-restrained. The GGVV (SEQ ID NO: 1) epitope emerges as a prediction for PDB structure 2MXU. For fibril structure 2MXU, 2 sequences bracketing GGVV (SEQ ID NO: 1) from the left and right, 37-41 GGVVI (SEQ ID NO: 8) and 36-40 VGGVV (SEQ ID NO: 6), are predicted using Collective Coordinates (end-caps not restrained and restrained respectively).

The residues 37-40 GGVV (SEQ ID NO: 1) emerge from 2 predictions, and so are treated as a consensus sequence between these two predictions. The Promis Gō method predicts an epitope consisting of amino acids 37-42 of A-beta comprising sequence GGVVIA (SEQ ID NO: 15) specifically for chains A and L of PDB 2XMU (end cap chains). For chains B,C,D,E,F,G,H,I,J and K, epitope GGVV (SEQ ID NO:1) is predicted using the end-caps not restrained 2MXU structure.

### III. Curvature of the Cyclic Peptide

The curvature profiles of the cyclic and linear peptide CGGGVVG (SEQ ID NO: 2), along with the curvature profile of the fibril 2XMU, are shown in FIG. 2G. Glycine residues G37 and G38 have different curvature than the linear peptide, but similar curvature to the fibril. The valine residue V39 has significantly higher curvature in the cyclic peptide compared to the curvature of V39 in either the linear peptide or the fibril. The valine residue 40V in the cyclic peptide has curvature distinct from either the linear peptide or fibril, with curvature intermediate between the linear peptide and fibril.

FIG. 2A plots the curvature for linear CGGGVVG (SEQ ID NO: 2) as obtained from different equilibrium simulation times. The legend shows several curves that start from 10 ns and continue to either 30 ns, 50 ns, 70 ns, or 90 ns. As simulation time is increased, the curvature values converge to the values reported above and in Table 1. Similar studies are shown in FIG. 2B for the cyclic peptide and FIG. 2C for the fibril. Panels D, E, and F show the convergence in the sum of the curvature values as a function of simulation time, for the linear, cyclic, and fibril conformations respectively. The degree of convergence indicates that the error bars are approximately 0.016 radian for the cyclic peptide, 0.05 radian for the linear peptide, and 0.01 radian for the fibril. It was observed that the curvature values converged after 70 ns for both linear and cyclic ensembles. The average curvature as a function of residue index for CGGGVVG (SEQ ID No: 2) is shown in Panel G where the linear peptide is in solid dark grey, the cyclic peptide in solid light grey and the fibril in dotted line. Numerical values of the curvature for residues 37G, 38G, 39V and 40V are given in Table 1. The curvature for both the linear and cyclic peptides is generally larger for GGVV (SEQ ID NO: 1) than the curvature of those residues in the fibril, though the values of the curvature for the linear and cyclic sequences are within the range of values of curvature in the fibril.

Curvature values for all residues in the peptide are obtained after averaging over the respective equilibrium ensembles. A point (x, y) in the linear, cyclic, or fibril-2MXU plots of Panels A, B, or C corresponds to the curvature of residues native residues 37-40, GGVV (SEQ ID NO: 1); residues outside this range in Panels A and B, i.e. 36 in Panel A, and 35, 36, and 41 in Panel B, correspond to non-native residues present in the linear and cyclic constructs respectively. Convergence is demonstrated by averaging over ensembles from 10 ns to increasing times 30 ns, 50 ns, 70 ns, and 90 ns.

For the plots in FIGS. 1-10 discussed herein, the data are obtained from equilibrium simulations in explicit solvent (TIP3P) using the Charmm27 force field. The simulation time and number of configurations for each ensemble are as follows. Cyclic peptide ensemble: simulation time 100 ns, containing 20000 frames; linear peptide ensemble: simulation time 100 ns, containing 20000 frames; 2M4J ensemble: 20 ns, containing 12000 frames.

Because the curvature of the cyclic epitope has a different profile than either the linear peptide or fibril, it is expected that the corresponding stretch of amino acids on an oligomer containing these residues would have a backbone orientation that is distinct from that in the fibril or monomer. However the degree of curvature would not be unphysical—values of curvature characterizing the cyclic peptide are obtained in several locations of the fibril.

Based on FIG. 2, the curvature values of residues 37G, 38 G, 39V and 40V are shown in Table 1 for the linear, cyclic and fibril (2MXU) peptides.

TABLE 1
Curvature value by residue

| | Linear | cyclic | 2MXU |
|---|---|---|---|
| 37G | 1.33 | 1.21 | 1.15 |
| 38G | 1.37 | 0.88 | 0.88 |
| 39V | 1.34 | 1.53 | 0.94 |
| 40V | 1.36 | 1.26 | 1.05 |

### IV. Dihedral Angle Distributions

Further computational support for the identification of an oligomer-selective epitope, is provided by both the side chain dihedral angle distributions, and the Ramachandran, ϕ and ψ distributions for the backbone dihedral angles in the cyclic peptide a proxy for an exposed epitope in the oligomer—are for some angles substantially different from the corresponding distributions in either the fibril or monomer.

The side-chain and backbone dihedral distributions were examined for residues 38G and 40V. Most dihedrals showed overlap between the cyclic form and other forms, although the dihedral angles shown for 38G show discrimination between the cyclic form and other forms. Percent overlap of distribution e.g. “linear” in distribution “cyclic” is obtained by dividing the angles into elements of 5°, then decreasing a cutoff in probability amplitude from infinity, until 90% of the cyclic distribution is above the cutoff, and 10% remains below. This defines one or more regions in the allowable angles. Percent of the linear distribution within this region was then found. The recipe is non-reciprocal and generally yields different numbers between pairs of distributions (e.g. 50.9% and 76.7% for linear/cyclic of 40V: O-C-Cα-Cβ).

The distributions of the O-C-C_{α}-HA1 and the O-C-C_{α}-HA2 dihedral angles for 38G are different for the cyclic peptide than for either the linear or the fibril. The distribution of the O-C-C_{α}-N dihedral angle for the cyclic peptide is similar to the linear peptide but different from the fibril. The distributions of the O-C-C_{α}-C_{β} dihedral angle for all three forms are similar (FIG. 3). In the following descriptions and FIGs, CA, Ca, or C_{α} are alternatively used to describe the C-alpha atom, and similarly for CB, Cb, and C_{β}, and so on.

From the dihedral distributions shown for 38G and 40V in FIG. 3. The probability that the linear peptide occupies a dihedral within the range of almost all (90%) of the cyclic peptide dihedral angles is as follows for the dihedral angles of 38G:
C-CA-CB-HA1: 4.4%
N-CA-CB-HA2: 35.8%
O-C-CA-N: 17.4%
   The probability that the linear peptide occupies a dihedral within the range of almost all (90%) of the cyclic peptide dihedral angles is as follows for the dihedral angle of 40V:
O-C-CA-CB: 50.9%
   Note that the accumulation of relatively small differences in individual dihedral angles can result in a large and significant difference in global conformation of the peptide, as described further in Example VIII below.

The probability that the peptide in the context of the fibril occupies a dihedral within the range of almost all (90%) of the cyclic peptide dihedral angles is as follows for the dihedral angles of 38G:
C-CA-CB-HA1: 2.6%
N-CA-CB-HA2: 1.1%
O-C-CA-N: 52%

The probability that the peptide in the context of the fibril occupies a dihedral within the range of almost all (90%) of the cyclic peptide dihedral angles is as follows for the dihedral angles of 40V:
O-C-CA-CB: 98.6%

Based on FIG. 3, Table 2 shows the percent overlap of dihedral angle distributions for backbone and side-chain angles of residues 38G and 40V in linear, cyclic and fibril (2MXU) forms relative to each other. E.g. Column 1 shows the percentage overlap between O-C-C_{α}-H_{α1} angle of 38G in the linear peptide and the same angle in the cyclic form.

TABLE 2
Percent overlap of dihedral angle distribution

| | linear in | 2MXU in | cyclic in | 2MXU in | linear in | cyclic in |
|---|---|---|---|---|---|---|
| | cyclic | cyclic | linear | linear | 2MXU | 2MXU |
| 38G: O-C- | 4.4% | 2.6% | 8.5% | 45.3% | 13.5% | 5.1% |
| CA-HA1 | | | | | | |
| 38G: O-C- | 35.8% | 1.1% | 38.2% | 44.6% | 12.3% | 0.1% |
| CA-HA2 | | | | | | |
| 38G: O-C- | 17.4% | 52% | 74.8% | 37.3% | 12.5% | 53% |
| CA-N | | | | | | |
| 40V: O-C- | 50.9% | 98.6% | 76.7% | 85.5% | 16.4% | 58.3% |
| CA-CB | | | | | | |

According to the above analysis of side chain and backbone dihedral angle distributions, residue 38G shows the largest discrepancy from the linear peptide and fibril ensembles. By these metrics, 38G may be a key residue on the epitope conferring conformational selectivity. Residue 40V shows smaller discrepancies, but may assist in conferring conformational selectivity.

Based on the data shown in FIG. 3, Table 3 lists the peak values of the dihedral angle distributions, for those dihedral angles whose distributions that show significant differences between the cyclic peptide and other species. Column 1 in Table 3 is the specific dihedral considered, column 2 is the peak value of the dihedral distribution for that angle in the context of the linear peptide CGGGVVG (SEQ ID NO: 2), column 3 is the peak value of the dihedral distribution for that angle in the context of the cyclic peptide CGGGVVG (SEQ ID NO: 2), column 4 is the difference of the peak values of the dihedral distributions for the linear and cyclic peptides, and column 5 is the peak value of the dihedral distribution for the peptide GGVV (SEQ ID NO: 1) in the context of the fibril structure 2MXU.

TABLE 3
Peak Values of the Dihedral Angle Distributions

| Dihedral Angle | linear | cyclic | 2MXU | Cyclic-linear |
|---|---|---|---|---|
| 38G: O-C-CA-HA1 | 62.5 | 122.5 | −147.5 | 60 |
| 38G: O-C-CA-HA2 | −57.5 | −112.5 | 87.5 | −55 |
| 38G: O-C-CA-N | 180 | 7.5 | −32.5 | −172.5 |
| 40V: O-C-CA-CB | 72.5, −72.5 | 57.5, −102.5 | 67.5 | −15, −30 |

### V. Entropy of the Side Chains

The side chain entropy of a residue may be approximately calculated from

$$S / {k}_{B} = - {\sum }_{i} \int d {φ}_{i} p ( {φ}_{i} ) ln p ( {φ}_{i} ) .$$

Where the sum is over all dihedral angles in a particular residue's side chain, and p(ϕᵢ) is the dihedral angle distribution, as analyzed above.

### Dissection of Entropy of Residue Side-Chain Moieties

The entropy of each dihedral angle was investigated in 37G, 38G, 39V and 40V. The entropy of the dihedral angles for each of the residues is plotted in FIG. 4 Panels A-D. The entropy for several dihedrals of 39V and 40V is reduced relative to the linear form, indicating a restricted pose for those angles in a conformation that tends to be distinct from the linear form and likely the monomer. Panel E plots the total side chain entropy (not including Ramachandran backbone angles) for residues G37, G38, V39, and V40 relative to the entropy of the fibril, e.g. ΔS for the cyclic peptide is S(cyclic)-S(fibril). This shows that entropy is increased relative to the fibril for the both the cyclic and linear peptides, but that the cyclic peptide has less entropy than the linear peptide. Panel F plots the total side chain plus backbone entropy for residues G37, G38, V39, and V40 relative to the entropy of the fibril. This shows again that entropy is increased relative to the fibril, but that the cyclic peptide has less entropy than the linear peptide, and so is more strongly constrained. Panel G again plots the total conformational entropy for residues G37, G38, V39, and V40 relative to the entropy of the linear monomer. This shows that confirmations present in the cyclic peptide are relatively rare in the linear peptide, and so would be unlikely to be sampled by chance. The probability to be in such a restricted set of conformations is approximately exp(−ΔS)≈0.001. The probability to be in the fibril conformation is enhanced by enthalpic compensation for the concomitant entropic loss.

### VI. Ramachandran Angles

The backbone orientation that the epitope exposes to an antibody differs depending on whether the peptide is in the linear, cyclic, or fibril form. This discrepancy can be quantified by plotting the Ramachandran angles phi and psi (or ϕ and ψ), along the backbone, for residue 38G in both the linear and cyclic peptides. FIG. 5 plots the phi and psi angles sampled in equilibrium simulations, for residue 38G in both linear and cyclic peptides consisting of sequence CGGGVG (SEQ ID No: 2), as well as GGVV (SEQ ID NO: 1) in the context of the fibril structure 2MXU. From FIG. 5 it can be seen that the distribution of backbone dihedral angles in the cyclic peptide is different from the distribution of dihedral angles sampled for either the linear peptide, and is more similar to the backbone angles for residues GGVV (SEQ ID NO: 1) in the context of the fibril structure 2MXU.

The probabilities of the Ramachandran angles of the residue 38G in the linear form overlapping with 90% of the Ramachandran angles in the cyclic form is 16%. The probabilities of the Ramachandran angles of the residue 38G in the fibril form overlapping with 90% of the Ramachandran angles in the cyclic form is 43%. There is a greater possibility that the Ramachandran angles distribution of a cyclic peptide to be similar to that of a fibril form. See Table 4.

TABLE 4
Overlap probabilities for Ramachandran angles of 38G

| | linear in | 2MXU in | cyclic in | 2MXU in | linear in | cyclic in |
|---|---|---|---|---|---|---|
| | cyclic | cyclic | linear | linear | 2MXU | 2MXU |
| 38G | 16% | 43% | 39% | 41% | 13% | 42% |

As a specific example, for residue 38G, Table 5 gives the peak (most-likely) values of the Ramachandran ϕ,ψ angles plotted in FIG. 5. The cyclic peptide distribution has peak values (most-likely values) at (ϕ,ψ)=((70°,180°), (−80°, 175°), (−85, −170°) and (75°, −180°) (there are four peaks). For the linear peptide, the most likely values are (ϕ,ψ)=(85°,5°), and (−85°,−10°) (there are two peaks) and for the fibril structure 2MXU, these most likely values are (ϕ,ψ)=(−60°,145°), (80°, 170°) and (70°,−180°). The difference in many of these peak dihedral angle values implies that antibodies selected for the cyclic epitope conformation will likely have lower affinity for the linear and fibril epitopes.

The peak values (most likely values) of the Ramachandran backbone ϕ,ψ distributions for 38G are given in Table 5. The first column in Table 5 gives the residue considered, which manifests two angles, phi and psi, indicated in parenthesis. The 2^{nd} column indicates the peak values of the Ramachandran phi/psi angles for 38G in the context of the linear peptide CGGGVVG (SEQ ID No:2), while the 3^{rd} column indicates the peak values of the Ramachandran phi/psi angles for 38G in the context of the cyclic peptide CGGGVVG (SEQ ID No:2), and the last column indicates the peak values of the Ramachandran phi/psi angles for 38G in the context of the fibril structure 2MXU.

TABLE 5
Peak values of distributions of backbone phi/psi angles

| | Peak values of distributions of G38 | | |
|---|---|---|---|
| | backbone phi/psi angles | | |
| | linear | cyclic | fibril |
| G38 | (85, 5) | (70, 180) (−80, 175) | (−60, 145) (80, 170) |
| | (−85, −10) | (−85, −170) (75, −180) | (70, −180) |

### VII. Solubility and Antigenicity of the Epitope

FIG. 6 plots the solvent accessible surface area (SASA). This shows that the SASA of residues GGVV (SEQ ID NO:1) in the cyclic peptide is increased over the fibril, indicating more solvent exposure would be accessible to antibody binding. The increase in exposure is most significant for residues V39 and V40. V39 is completely buried in the fibril, and is most exposed in the cyclic peptide.

Residue V39, and to a lesser extent V40, have the most likelihood of differential exposure and availability for antibody binding, as compared to those residues in the conformation of GGVV (SEQ ID NO:1) in the fibril structure.

VIII. The Ensemble of Cyclic Peptide Conformations Clusters Differently than the Ensemble of Either Linear or Fibril Conformations

Definitive evidence that the sequence GGVV (SEQ ID No:1) displays a different conformation in the context of the cyclic peptide than in the linear peptide can be seen by using standard structural alignment metrics between conformations, and then implementing clustering analysis. Equilibrium ensembles of conformations are obtained for the linear and cyclic peptides CGGGVVG (SEQ ID No: 2), as well as the full-length fibril in the structure corresponding to PDB ID 2MXU. Snapshots of conformations from these ensembles for residues GGVV (SEQ ID NO: 1) are collected and then structurally aligned to the centroids of the largest cluster of the cyclic peptide ensemble, the largest cluster of the linear peptide ensemble, and the largest cluster of GGVV (SEQ ID NO: 1) in the fibril ensemble; the three values of the root mean squared deviation (RMSD) are then recorded and plotted. The clustering is performed here by the maxcluster algorithm (http://www.sbg.bio.ic.ac.uk/maxcluster). The 3 corresponding RMSD values for the linear, cyclic, and fibril ensembles are plotted as a 3-dimensional scatter plot in FIG. 9.

Table 6 shows the percentage overlap of the RMSD scatter plot of the linear, cyclic and fibril (2MXU) peptide conformations. Column 1 shows the percentage overlap between the linear form and the cyclic form is quite small, only 3%.

TABLE 6
Percentage overlap of RMSD clustering

| linear in | 2MXU in | cyclic in | 2MXU in | linear in | Cyclic in | linear in | linear in | linear in |
|---|---|---|---|---|---|---|---|---|
| cyclic | cyclic | linear | linear | 2MXU | 2MXU | 2LMP | 2M4J | 2LMN |
| 3% | 0 | 10% | 0.7% | 0.04% | 0 | 2% | 10% | 8.5% |

It is evident from FIG. 9 that the 3 ensembles cluster differently from each other. In particular the cyclic peptide structural ensemble is distinct from either the linear or fibril ensembles, implying that antibodies specific to the cyclic peptide epitope may have low affinity to the conformations presented in the linear or fibril ensembles. An antibody raised to the cyclic peptide could be conformationally selective and preferentially bind oligomeric forms over either the linear or fibril conformations of Abeta. Such an antibody would also be unlikely to preferentially bind the various strains of Abeta40, because of the charged termini present for these strains. The distinction between the ensembles occurs in spite of the overlap between several side chain and backbone dihedral angle distributions; the numerous often small differentiating features described above lead to globally different conformational distributions.

The overlap between the ensembles was calculated as follows. The fraction (percent) of the linear ensemble that overlaps with the cyclic ensemble is obtained by first dividing the volume of this 3-dimensional RMSD space up into cubic elements of length 0.1 Angstrom. Then a “cutoff density” of points in the cyclic distribution is found such that the cubes with cyclic distribution density equal to or higher than the cutoff density contain 90% of the cyclic distribution. This defines a volume (which may be discontiguous) that gives the characteristic volume containing the cyclic distribution and removes any artifacts due to outliers. Then the fraction of points from the linear distribution that are within this region is found. With this method, it is possible to find the overlapping percentages for fibril in linear, cyclic in linear, etc. Generally, very low overlapping is observed.

The numeric overlapping percentage obtained by the above method is given in Table 6. In particular, the cyclic peptide and the fibril peptide 2MXU have 0% overlap. By the above recipe, the overlap of the linear distribution with the cyclic distribution is 3%, and the overlap of the cyclic distribution with the linear peptide distribution is 10%.

FIG. 9 Panels D-G illustrate the convergence of the ensemble overlap values. FIG. 9D shows that the linear and fibril ensembles have an overlap that has converged to less than 1%. FIG. 9E shows that the linear ensemble overlaps with the cyclic ensemble by a converged value of about 3%. FIG. 9F shows that the linear ensemble overlaps with the fibril ensemble by a converged value of less than 0.04%. FIG. 9G shows that the cyclic ensemble overlaps with the linear ensemble by a converged value of about 10%.

Two views of a representative conformation of GGVV (SEQ ID NO. 1) from the cyclic peptide ensemble, constituting the centroid of the largest cluster from the cyclic peptide ensemble of structures, are shown in FIG. 7 Panel A in black. As well, the most representative conformation in the linear peptide ensemble, constituting the centroid of the largest cluster, is shown in white in FIG. 7, superimposed on the cyclic peptide shown in black by optimally aligning them using RMSD, to make explicit their different orientations. FIG. 7 Panel B shows the corresponding centroid conformations for the cyclic peptide and linear peptide for sequence CGGGVVG (SEQ ID No. 2), again optimally superimposed by aligning with respect to RMSD. The black colored conformation is the centroid of the largest cluster of the cyclic peptide, and so best represents the typical conformation of the cyclic peptide. The white colored conformation is the centroid of the largest cluster of the linear peptide, which is aligned to the cyclic conformation. The superimposed aligned structures show that different dihedral angles and overall epitope conformations tend to be preferred for the linear and cyclic peptides.

Table 7 lists values of the Ramachandran backbone and side chain dihedral angles occupied by G37, G38, V39, and V40 in the centroid structures of the cyclic peptide ensemble, the linear peptide ensemble, and the fibril ensemble; cyclic and linear centroid conformations are plotted in FIG. 7. The centroid structures exhibit several dihedral angles that are substantially different between the cyclic conformation and either linear or fibril conformations. Column 1 of Table 7 gives the residue of interest, column 2 lists the dihedral angle of interest, column 3 gives the value of the dihedral angle in the centroid structure of the cyclic ensemble, column 4 gives the value of the dihedral in the linear ensemble centroid, column 5 gives the value of the dihedral in the fibril ensemble centroid, column 6 gives the difference in dihedral angle between the cyclic centroid and linear centroid, column 7 gives the difference in dihedral angle between the cyclic centroid and fibril centroid. Many of the dihedral angles in table 7 are constrained to not be a large, e.g. dihedrals terminating in 1HG1, 2HG1, 3HG1, 1HG2, 2HG2, and 3HG2 cannot be larger than 120°, however it is apparent that many of the cyclic dihedral angles are significantly different then the corresponding dihedral angles in the linear or fibril centroids.

TABLE 7
Dihedral angles in the centroid structures of the linear,
cyclic, and fibril ensembles, along with their differences.

| Residue | Dihedral | cyclic | linear | fibril(2MXU) | cyclic-linear | cyclic-fibril |
|---|---|---|---|---|---|---|
| G37 | C-N-CA-C(phi) | 109.06 | 67.42 | 130.02 | 41.64 | −20.96 |
| | N-CA-C-N(psi) | −113.89 | 24.79 | 0.39 | −138.67 | −114.28 |
| | HA1-CA-N-HN | 52.08 | 14.20 | 71.49 | 37.88 | −19.41 |
| | HA2-CA-N-HN | 173.42 | 131.97 | −169.73 | 41.45 | −16.85 |
| | C-CA-N-HN | −60.90 | −104.01 | −56.48 | 43.11 | −4.42 |
| | O-C-CA-HA1 | −49.10 | 93.49 | 49.22 | −142.59 | −98.32 |
| | O-C-CA-HA2 | −168.92 | −23.86 | −71.17 | −145.06 | −97.75 |
| | O-C-CA-N | 66.84 | −149.48 | 176.39 | −143.68 | −109.55 |
| G38 | C-N-CA-C(phi) | −75.42 | 71.58 | 98.04 | −147.00 | −173.46 |
| | N-CA-C-N(psi) | −174.30 | 9.91 | 116.88 | 175.78 | 68.81 |
| | HA1-CA-N-HN | −20.86 | 7.89 | 32.36 | −28.75 | −53.22 |
| | HA2-CA-N-HN | −138.75 | 134.36 | 153.69 | 86.88 | 67.55 |
| | C-CA-N-HN | 91.73 | −107.85 | −85.21 | −160.42 | 176.95 |
| | O-C-CA-N | 6.47 | −171.54 | −75.53 | 178.01 | 82.00 |
| | O-C-CA-HA1 | 118.97 | 70.69 | 164.18 | 48.28 | −45.21 |
| | O-C-CA-HA2 | −127.46 | −56.68 | 42.99 | −70.78 | −170.45 |
| V39 | C-N-CA-C(phi) | −63.81 | −68.62 | −88.92 | 4.81 | 25.11 |
| | N-CA-C-N(psi) | −52.30 | −44.80 | 134.12 | −7.50 | 173.58 |
| | HA-CA-N-HN | −116.20 | −141.02 | −162.82 | 24.82 | 46.62 |
| | C-CA-N-HN | 120.23 | 104.11 | 77.88 | 16.12 | 42.35 |
| | CB-CA-N-HN | 3.08 | −19.64 | −54.49 | 22.72 | 57.57 |
| | O-C-CA-N | 129.72 | 143.36 | −49.90 | −13.64 | 179.62 |
| | O-C-CA-HA | 4.96 | 25.26 | −168.83 | −20.31 | 173.79 |
| | O-C-CA-CB | −110.62 | −88.75 | 80.38 | −21.87 | 168.99 |
| | N-CA-CB-HB | 57.27 | 76.60 | 62.11 | −19.34 | −4.85 |
| | N-CA-CB-CG2 | −62.02 | −42.04 | −62.16 | −19.98 | 0.14 |
| | N-CA-CB-CG1 | −178.13 | −164.35 | −173.85 | −13.78 | −4.29 |
| | HA-CA-CB-HB | −178.04 | −162.19 | 174.46 | −15.84 | 7.50 |
| | HA-CA-CB-CG2 | 62.67 | 79.16 | 50.19 | −16.49 | 12.48 |
| | HA-CA-CB-CG1 | −53.44 | −43.15 | −61.50 | −10.29 | 8.06 |
| | C-CA-CB-HB | −57.61 | −52.44 | −70.71 | −5.16 | 13.11 |
| | C-CA-CB-CG2 | −176.90 | −171.09 | 165.02 | −5.81 | 18.09 |
| | C-CA-CB-CG1 | 66.99 | 66.60 | 53.33 | 0.39 | 13.66 |
| | HB-CB-CG2- | 176.19 | −170.47 | −162.57 | −13.34 | −21.24 |
| | 1HG2 | | | | | |
| | HB-CB-CG2- | 57.97 | 74.59 | 86.50 | −16.62 | −28.52 |
| | 3HG2 | | | | | |
| | HB-CB-CG2- | −63.47 | −38.11 | −45.22 | −25.36 | −18.25 |
| | 2HG2 | | | | | |
| | CA-CB-CG2- | −63.95 | −53.07 | −42.83 | −10.88 | −21.12 |
| | 1HG2 | | | | | |
| | CA-CB-CG2- | 177.84 | −168.01 | −153.77 | −14.16 | −28.39 |
| | 3HG2 | | | | | |
| | CA-CB-CG2- | 56.39 | 79.29 | 74.51 | −22.90 | −18.12 |
| | 2HG2 | | | | | |
| | CG1-CB-CG2- | 55.90 | 70.75 | 73.59 | −14.85 | −17.69 |
| | 1HG2 | | | | | |
| | CG1-CB-CG2- | −62.31 | −44.18 | −37.35 | −18.13 | −24.96 |
| | 3HG2 | | | | | |
| | CG1-CB-CG2- | 176.24 | −156.89 | −169.07 | −26.87 | −14.69 |
| | 2HG2 | | | | | |
| | HB-CB-CG1- | 177.16 | 163.64 | 158.98 | 13.52 | 18.18 |
| | 1HG1 | | | | | |
| | HB-CB-CG1- | −61.94 | −76.02 | −73.72 | 14.08 | 11.79 |
| | 2HG1 | | | | | |
| | HB-CB-CG1- | 60.25 | 52.24 | 38.49 | 8.00 | 21.76 |
| | 3HG1 | | | | | |
| | CA-CB-CG1- | −62.47 | −64.03 | −82.41 | 1.56 | 19.94 |
| | 1HG1 | | | | | |
| | CA-CB-CG1- | 54.44 | 47.36 | 38.07 | 7.08 | 16.37 |
| | 2HG1 | | | | | |
| | CA-CB-CG1- | 175.34 | 167.70 | 165.37 | 7.64 | 9.97 |
| | 3HG1 | | | | | |
| | CG2-CB-CG1- | 177.67 | 169.53 | 163.55 | 8.14 | 14.12 |
| | 1HG1 | | | | | |
| | CG2-CB-CG1- | −65.42 | −79.07 | −75.96 | 13.66 | 10.54 |
| | 2HG1 | | | | | |
| | CG2-CB-CG1- | 55.48 | 41.27 | 51.34 | 14.22 | 4.14 |
| | 3HG1 | | | | | |
| V40 | C-N-CA-C(phi) | −112.14 | −82.13 | −136.24 | −30.01 | 24.10 |
| | N-CA-C-N(psi) | 113.58 | −41.00 | 117.93 | 154.58 | −4.35 |
| | CB-CA-N-HN | −57.38 | −16.74 | −77.95 | −40.64 | 20.57 |
| | C-CA-N-HN | 64.57 | 102.44 | 47.57 | −37.87 | 17.00 |
| | HA-CA-N-HN | −169.89 | −133.28 | 156.66 | −36.61 | 33.44 |
| | N-CA-CB-HB | 69.06 | 76.98 | 57.77 | −7.92 | 11.29 |
| | N-CA-CB-CG1 | −165.89 | −162.61 | 176.14 | −3.29 | 17.96 |
| | N-CA-CB-CG2 | −48.00 | −42.56 | −66.59 | −5.44 | 18.59 |
| | C-CA-CB-HB | −52.96 | −43.72 | −63.28 | −9.24 | 10.32 |
| | C-CA-CB-CG1 | 72.09 | 76.69 | 55.10 | −4.60 | 16.99 |
| | C-CA-CB-CG2 | −170.01 | −163.26 | 172.37 | −6.75 | 17.62 |
| | HA-CA-CB-HB | −173.18 | −164.47 | −177.97 | −8.71 | 4.79 |
| | HA-CA-CB-CG1 | −48.13 | −44.06 | −59.60 | −4.07 | 11.47 |
| | HA-CA-CB-CG2 | 69.77 | 75.99 | 57.68 | −6.22 | 12.09 |
| | O-C-CA-CB | 54.24 | −108.00 | 68.53 | 162.23 | −14.29 |
| | O-C-CA-N | −71.93 | 132.45 | −57.00 | 155.62 | −14.93 |
| | O-C-CA-HA | 165.36 | 9.40 | −171.29 | 155.97 | −23.35 |
| | HB-CB-CG1- | −153.06 | −159.72 | 179.67 | 6.65 | 27.27 |
| | 2HG1 | | | | | |
| | HB-CB-CG1- | −33.99 | −41.87 | −57.48 | 7.87 | 23.48 |
| | 3HG1 | | | | | |
| | HB-CB-CG1- | 79.81 | 81.48 | 60.91 | −1.67 | 18.90 |
| | 1HG1 | | | | | |
| | CG2-CB-CG1- | −38.95 | −47.76 | −66.86 | 8.82 | 27.91 |
| | 2HG1 | | | | | |
| | CG2-CB-CG1- | 80.12 | 70.08 | 56.00 | 10.04 | 24.12 |
| | 3HG1 | | | | | |
| | CG2-CB-CG1- | −166.07 | −166.57 | 174.38 | 0.50 | 19.54 |
| | 1HG1 | | | | | |
| | CA-CB-CG1- | 79.09 | 73.72 | 52.30 | 5.37 | 26.79 |
| | 2HG1 | | | | | |
| | CA-CB-CG1- | −161.84 | −168.43 | 175.16 | 6.59 | 23.00 |
| | 3HG1 | | | | | |
| | CA-CB-CG1- | −48.03 | −45.08 | −66.45 | −2.95 | 18.42 |
| | 1HG1 | | | | | |
| | HB-CB-CG2- | −177.08 | 171.03 | −155.36 | 11.89 | −21.73 |
| | 3HG2 | | | | | |
| | HB-CB-CG2- | 63.11 | 54.85 | 83.11 | 8.27 | −20.00 |
| | 2HG2 | | | | | |
| | HB-CB-CG2- | −51.70 | −62.09 | −35.11 | 10.39 | −16.59 |
| | 1HG2 | | | | | |
| | CG1-CB-CG2- | −54.58 | −59.12 | −27.99 | 4.54 | −26.59 |
| | 3HG2 | | | | | |
| | CG1-CB-CG2- | −169.39 | −176.06 | −146.21 | 6.67 | −23.18 |
| | 2HG2 | | | | | |
| | CG1-CB-CG2- | 65.23 | 57.06 | 93.54 | 8.17 | −28.32 |
| | 1HG2 | | | | | |
| | CA-CB-CG2- | −173.34 | −178.12 | −146.23 | 4.79 | −27.11 |
| | 3HG2 | | | | | |
| | CA-CB-CG2- | 71.85 | 64.94 | 95.56 | 6.91 | −23.70 |
| | 2HG2 | | | | | |
| | CA-CB-CG2- | −53.53 | −61.94 | −24.69 | 8.41 | −28.84 |
| | 1HG2 | | | | | |

FIG. 8 again shows the centroid structures for the cyclic, linear, and 2M4J fibril ensembles. The surface area profile, which would be presented to an antibody, is different between the centroid conformations. Specifically, the 2M4J terminates at residue V40, so has a charged carboxyl terminus. Thus antibodies raised to this region in A-beta 40 will be unlikely to bind cyclic GGVV (SEQ ID NO: 1), and conversely, antibodies raised to cyclic GGVV (SEQ ID NO: 1) will be unlikely to bind this region in A-beta 40.

FIG. 10 shows that the cyclic ensemble does not overlap with any of the other strains of A-beta fibril. Specifically, the overlap between the cyclic peptide ensembles distribution and fibril distributions is zero. FIG. 10A shows the result for PDB 2M4J, FIG. 10B for PDB 2LMN, and FIG. 10C for PDB 2LMP.

### Example 3

### Cyclic Compound Construction Comprising a Conformationally Constrained Epitope

Peptides comprising GGVV (SEQ ID NO: 1) such as Cyclo(CGGGVVG) (SEQ ID NO: 2) can be cyclized head to tail.

A linear peptide comprising GGVV (SEQ ID NO: 1) and a linker, preferably comprising 2, 3, or 4 amino acids and/or PEG units, can be synthesized using known methods such as Fmoc based solid phase peptide synthesis alone or in combination with other methods. PEG molecules can be coupled to amine groups at the N terminus for example using coupling chemistries described in Hamley 2014 [6] and Roberts et al 2012 [7], each incorporated herein by reference. The linear peptide compound may be cyclized by covalently bonding 1) the amino terminus and the carboxy terminus of the peptide+linker to form a peptide bond (e.g. cyclizing the backbone), 2) the amino or carboxy terminus with a side chain in the peptide+linker or 3) two side chains in the peptide+linker.

The bonds in the cyclic compound may be all regular peptide bonds (homodetic cyclic peptide) or include other types of bonds such as ester, ether, amide or disulfide linkages (heterodetic cyclic peptide).

Peptides may be cyclized by oxidation of thiol- or mercaptan-containing residues at the N-terminus or C-terminus, or internal to the peptide, including for example cysteine and homocysteine. For example two cysteine residues flanking the peptide may be oxidized to form a disulphide bond. Oxidative reagents that may employed include, for example, oxygen (air), dimethyl sulphoxide, oxidized glutathione, cystine, copper (II) chloride, potassium ferricyanide, thallium(III) trifluro acetate, or other oxidative reagents such as may be known to those of skill in the art and used with such methods as are known to those of skill in the art.

Methods and compositions related to cyclic peptide synthesis are described in US Patent Publication 2009/0215172. US Patent publication 2010/0240865, US Patent Publication 2010/0137559, and U.S. Pat. No. 7,569,541 describe various methods for cyclization. Other examples are described in PCT Publication WO01/92466, and Andreu et al., 1994. Methods in Molecular Biology 35:91-169.

More specifically, a cyclic peptide comprising the GGVV (SEQ ID NO: 1) epitope can be constructed by adding a linker comprising a spacer with cysteine residues flanking and/or inserted in the spacer. The peptide can be structured into a cyclic conformation by creating a disulfide linkage between the non-native cysteines residues added to the N- and C-termini of the peptide. It can also be synthesized into a cyclic compound by forming a peptide bond between the N- and C-termini amino acids (e.g. head to tail cyclization).

Peptide synthesis is performed by CPC Scientific Inc. (Sunnyvale Calif., USA) following standard manufacturing procedures.

For example Cyclo(CGGGVVGC) (SEQ ID NO: 12) cyclic peptide comprising the conformational epitope GGVV (SEQ ID NO: 1) is constructed in a constrained cyclic conformation using a disulfide linkage between cysteine residues added to the N- and C-termini of a peptide comprising GGVV (SEQ ID NO: 1). Two non-native cysteine residues were added to GGGVV (SEQ ID NO: 11) one at the C-terminus and one at the N-terminus. The two cysteines are oxidized under controlled conditions to form a disulfide bridge or reacted head to tail to produce a peptide bond.

As described above, the structure of the cyclic peptide was designed to mimic the conformation and orientation of the amino acid backbone and side chains of GGVV (SEQ ID NO: 1) in A-beta oligomer.

### Cyclo(CGGGVVG) (SEQ ID NO: 2)

Cyclo(CGGGVVG) (SEQ ID NO: 2) was synthesized using the following method (CPC Scientific Inc, Sunnyvale Calif.). The protected linear peptide was synthesized by standard conventional Fmoc-based solid-phase peptide synthesis on 2-chlorotrityl chloride resin, followed by cleavage from the resin with 30% HFIP/DCM. Protected linear peptide was cyclized to the corresponding protected cyclic peptide by using EDC. HCl/HOBt/DIEA in DMF at low concentration. The protected cyclic peptide was deprotected by TFA to give crude cyclic peptide and the crude peptide was purified by RP HPLC to give pure cyclic peptide after lyophilize.

Cyclo(CGGGVVG) (SEQ ID NO: 2) can be prepared by amide condensation of the linear peptide CGGGVVG (SEQ ID NO: 2).

Cyclo(C-PEG2-GGVVG) (SEQ ID NO: 3) can be prepared by amide condensation of the linear compound C-PEG2-GGVVG (SEQ ID NO: 3).

Cyclo(CGGGVV-PEG2) (SEQ ID NO: 4) can be prepared by amide condensation of the linear compound CGGGVV-PEG2 (SEQ ID NO: 4).

Linear(CGGGVVG) (SEQ ID NO: 2) was prepared (CPC Scientific Inc, Sunnyvale Calif.) The protected linear peptide was synthesized by standard conventional Fmoc-based solid-phase peptide synthesis on Fmoc-Gly-Wang resin, then the protected peptide was cleaved by TFA to give crudepeptide and the crude peptide was purified by RP HPLC to give pure peptide after lyophilize, and which was used to conjugate BSA.

### Immunogen Construction

The cyclic compound Cyclo(CGGGVVG) (SEQ ID NO: 2) was synthesized as described above and then conjugated to BSA and/or KLH (CPC Scientific Inc, Sunnyvale Calif.). BSA or KLH was re-activated by SMCC in PBS buffer, then a solution of the pure peptide in PBS buffer was added to the conjugation mixture, the conjugation mixture was stirred at room temperature for 2 h. Then the conjugation mixture was lyophilized after dialysis to give the conjugation product.

### Example 4

### Antibody Generation and Selection

A conformational constrained compound optionally a cyclic compound such as a cyclic peptide comprising GGVV (SEQ ID NO: 1) such as cyclo(CGGGVVG) (SEQ ID NO: 2) peptide is linked to Keyhole Limpet Hemocyanin (KLH). The cyclopeptide is sent for mouse monoclonal antibody production (ImmunoPrecise Antibodies LTD (Victoria BC, Canada), following protocols approved by the Canadian Council on Animal Care. Mouse sera are screened using either the conformational peptide used for producing the antibodies or a related peptide e.g. cyclo(CGGGW) (SEQ ID NO: 2), linked to BSA.

Hybridomas were made using an immunogen comprising cyclo(CGGGVVG) (SEQ ID NO: 2) as further described in Example 6. Hybridoma supernatants were screened by ELISA and SPR for preferential binding to cyclo(CGGGVVG) (SEQ ID NO: 2) peptide vs linear peptide as described herein. Positive IgG-secreting clones are subjected to large-scale production and further purification using Protein G.

### Example 5

### Assessing Binding or Lack Thereof to Plaques/Fibrils

Immunohistochemistry can be performed on fresh frozen human brain sections, or frozen human brain sections, post fixed in 10% formalin. Endogenous peroxidase activity can be quenched using 0.5% hydrogen peroxide in methanol for 20 min. Antigen retrieval can be achieved using sodium citrate pH 6.0 and steam heating for 25 min followed by cooling at room temperature (RT) for 30 min. After stabilization in TBS for 5-7 min, sections are treated by 70% formic acid for 15 min at RT, and then washed 3×15 min in TBS. In a humidified chamber, non-specific staining is blocked by incubation with serum-free protein blocking reagent (Dako Canada Inc., Mississauga, ON, Canada) for 1 h.

For immunostaining, antibodies described herein, positive control 6E10 (1 μg/ml) and isotype controls such as IgG1, IgG2a, IgG2b and IgG3 (1 μg/ml, Abcam) are used as primary antibodies. Sections are incubated overnight at 4° C., and washed 3×5 min in TBS-T. Anti-mouse IgG Horseradish Peroxidase conjugated (1:1000, ECL) is applied to sections and incubated 45 min, then washed 3×5 min in TBS-T. DAB chromogen reagent (Vector Laboratories, Burlington ON, Canada) is applied and sections rinsed with distilled water when the desired level of target to background staining is achieved. Sections are counterstained with Mayer's haematoxylin, dehydrated and cover slips were applied. Slides are examined under a light microscope (Zeiss Axiovert 200M, Carl Zeiss Canada, Toronto ON, Canada) and representative images captured at 50, 200 and 400× magnification using a Leica DC300 digital camera and software (Leica Microsystems Canada Inc., Richmond Hill, ON).

### Example 6

### Methods and Materials

### Immunogen

Cyclic and linear peptides were generated at CPC Scientific, Sunnyvale, Calif., USA. Peptides were conjugated to KLH (for immunizing) and BSA (for screening) using a trifluoroacetate counter ion protocol. Peptides were desalted and checked by MS and HPLC and deemed 95% pure. Peptides were shipped to IPA for use in production of monoclonal antibodies in mouse.

### Antibodies

A number of hybridomas and monoclonal antibodies were generated to cyclo(CGGGVVG) (SEQ ID NO: 2) linked to Keyhole Limpet Hemocyanin (KLH).

Fifty day old female BALB/c mice (Charles River Laboratories, Quebec) were immunized. A series of subcutaneous aqueous injections containing antigen but no adjuvant were given over a period of 19 days. Mice were immunized with 100 μg per mouse per injection of a 0.5 mg/mL solution in sterile saline of cyclic peptide-KLH. Mice were housed in a ventilated rack system from Lab Products. All 4 mice were euthanized on Day 19 and lymphocytes were harvested for hybridoma cell line generation.

### Fusion/Hybridoma Development

Lymphocytes were isolated and fused with murine SP2/0 myeloma cells in the presence of poly-ethylene glycol (PEG 1500). Fused cells were cultured using HAT selection. This method uses a semi-solid methylcellulose-based HAT selective medium to combine the hybridoma selection and cloning into one step. Single cell-derived hybridomas grow to form monoclonal colonies on the semi-solid media. 10 days after the fusion event, resulting hybridoma clones were transferred to 96-well tissue culture plates and grown in HT containing medium until mid-log growth was reached (5 days).

### Hybridoma Analysis (Screening)

Tissue culture supernatants from the hybridomas were tested by indirect ELISA on screening antigen (cyclic peptide-BSA) (Primary Screening) and probed for both IgG and IgM antibodies using a Goat anti-IgG/IgM(H&L)-HRP secondary and developed with TMB substrate. Clones>0.2 OD in this assay were taken to the next round of testing. Positive cultures were retested on screening antigen to confirm secretion and on an irrelevant antigen (Human Transferrin) to eliminate non-specific mAbs and rule out false positives. All clones of interest were isotyped by antibody trapping ELISA to determine if they are IgG or IgM isotype. All clones of interest were also tested by indirect ELISA on other cyclic peptide-BSA conjugates as well as linear peptide-BSA conjugates to evaluate cross-reactivity.

Mouse hybridoma antibodies were screened by Indirect ELISA using cyclo(CGGGVVG) (SEQ ID NO: 2) conjugated to BSA.

### ELISA Antibody Screening

Briefly, the ELISA plates were coated with 0.1 ug/well cyclo(CGGGVVG)-conjugated-BSA (SEQ ID NO: 2) at 100 uL/well in carbonate coating buffer (pH 9.6) O/N at 4 C and blocked with 3% skim milk powder in PBS for 1 hour at room temperature. Primary Antibody: Hybridoma supernatant at 100 uL/well incubated for 1 hour at 37 C with shaking. Secondary Antibody 1:10,000 Goat anti-mouse IgG/IgM(H+L)-HRP at 100 uL/well in PBS-Tween for 1 hour at 37 C with shaking. All washing steps were performed for 30 mins with PBS-Tween. The substrate 3,3′,5,5′-tetramethylbenzidine (TMB) was added at 50 uL/well, developed in the dark and stopped with equal volume 1M HCl.

Positive clones were selected for further testing. Positive clones of mouse GGVV (SEQ ID NO: 1) hybridomas were tested for reactivity to cyclo(CGGGVVG) (SEQ ID NO: 2) conjugated BSA and human transferrin (HT) by indirect ELISA. Plates were coated with 1) 0.1 ug/well cyclo(CGGGVVG)-conjugated-BSA (SEQ ID NO: 2) at 100 uL/well in carbonate coating buffer (pH 9.6) O/N at 4 C; or 2) 0.25 ug/well HT Antigen at 50 uL/well in dH2O O/N at 37 C. Primary Antibody: Hybridoma supernatant at 100 uL/well incubated for 1 hour at 37 C with shaking. Secondary Antibody 1:10,000 Goat anti-mouse IgG/IgM(H+L)-HRP at 100 uL/well in PBS-Tween for 1 hour at 37 C with shaking. All washing steps were performed for 30 mins with PBS-Tween. The substrate 3,3′,5,5′-tetramethylbenzidine (TMB) was added at 50 uL/well, developed in the dark and stopped with equal volume 1M HCl.

### ELISA Cyclo vs Linear CGGGVVG (SEQ ID NO:2) Compound Selectivity

ELISA plates were coated with 1) 0.1 ug/well cyclo(CGGGVVG)-conjugated-BSA (SEQ ID NO: 2) at 100 uL/well in carbonate coating buffer (pH 9.6) O/N at 4 C; 2)) 0.1 ug/well linear CGGGVVG-conjugated-BSA (SEQ ID NO: 2) at 100 uL/well in carbonate coating buffer (pH 9.6) O/N at 4 C; or 3) 0.1 ug/well Negative-Peptide at 100 uL/well in carbonate coating buffer (pH 9.6) O/N at 4 C. Primary Antibody: Hybridoma supernatant at 100 uL/well incubated for 1 hour at 37 C with shaking. Secondary Antibody 1:10,000 Goat anti-mouse IgG/IgM(H+L)-HRP at 100 uL/well in PBS-Tween for 1 hour at 37 C with shaking. All washing steps were performed for 30 mins with PBS-Tween. The substrate TMB was added at 50 uL/well, developed in the dark and stopped with equal volume 1M HCl.

### Isotyping

The hybridoma antibodies were isotyped using antibody trap experiments. Trap plates were coated with 1:10,000 Goat anti-mouse IgG/IgM(H&L) antibody at 100 uL/well carbonate coating buffer pH9.6 overnight at 4 C. No blocking step was used. Primary antibody (hybridoma supernatants) was added (100 ug/mL). Secondary Antibody 1:5,000 Goat anti-mouse IgGy-HRP or 1:10,000 Goat anti-mouse IgMp-HRP at 100 uL/well in PBS-Tween for 1 hour at 37 C with shaking. All washing steps were performed for 30 mins with PBS-Tween. The substrate TMB was added at 50 uL/well, developed in the dark and stopped with equal volume 1M HCl.

### SPR Binding Assays—Primary and Secondary Screens

### SPR Analysis of Antibody Binding to Abeta Monomers and Oligomers

A-beta Monomer and Oligomer Preparation Recombinant A-beta40 and 42 peptides (California Peptide, Salt Lake City Utah, USA) were dissolved in ice-cold hexafluoroisopropanol (HFIP). The HFIP was removed by evaporation overnight and dried in a SpeedVac centrifuge. To prepare monomers, the peptide film was reconstituted in DMSO to 5 mM, diluted further to 100 μM in dH2O and used immediately. Oligomers were prepared by diluting the 5 mM DMSO peptide solution in phenol red-free F12 medium (Life Technologies Inc., Burlington ON, Canada) to a final concentration of 100 μM and incubated for 24 hours to 7 days at 4° C.

SPR Analysis All SPR measurements were performed using a Molecular Affinity Screening System (MASS-1) (Sierra Sensors GmbH, Hamburg, Germany), an analytical biosensor that employs high intensity laser light and high speed optical scanning to monitor binding interactions in real time. The primary screening of tissue culture supernatants was performed using an SPR direct binding assay, whereby BSA-conjugated peptides, A-Beta42 Monomer and A-beta42 Oligomer are covalently immobilized on individual flow cells of a High Amine Capacity (HAC) sensorchip (Sierra Sensors GmbH, Hamburg, Germany) and antibodies flowed over the surface. Protein G purified mAbs were analyzed in a secondary screen using an SPR indirect (capture) binding assay, whereby the antibodies were captured on a protein A-derivatized sensorchip (XanTec Bioanalytics GmbH, Duesseldorf, Germany) and A-Beta40 Monomer, A-beta42 Oligomer, soluble brain extracts and cerebrospinal fluid flowed over the surface. The specificity of the antibodies was verified in an SPR direct binding assay by covalently immobilizing A-Beta42 Monomer and A-beta42 Oligomer on individual flow cells of a HAC sensorchip and flowing purified mAbs.

### SPR Analysis of Soluble Brain Extracts and CSF Samples

Soluble brain extract and CSF Preparation Human brain tissues and CSFs were obtained from patients assessed at the UBC Alzheimer's and Related Disorders Clinic. Clinical diagnosis of probable AD is based on NINCDS-ADRDA criteria [5]. CSFs are collected in polypropylene tubes, processed, aliquoted into 100 μL polypropylene vials, and stored at −80° C. within 1 hour after lumbar puncture.

Homogenization: Human brain tissue samples were weighed and subsequently submersed in a volume of fresh, ice cold TBS (supplemented with EDTA-free protease inhibitor cocktail from Roche Diagnostics, Laval QC, Canada) such that the final concentration of brain tissue is 20% (w/v). Tissue is homogenized in this buffer using a mechanical probe homogenizer (3×30 sec pulses with 30 sec pauses in between, all performed on ice). TBS homogenized samples are then subjected to ultracentrifugation (70,000×g for 90 min). Supernatants are collected, aliquoted and stored at −80° C. The protein concentration of TBS homogenates is determined using a BCA protein assay (Pierce Biotechnology Inc, Rockford Ill., USA).

SPR Analysis Brain extracts from 4 AD patients and 4 age-matched controls, and CSF samples from 9 AD patients and 9 age-matched controls were pooled and analyzed. Purified mAbs were captured on separate flow cells of a protein A-derivatized sensor chip and diluted samples injected over the surfaces for 180 seconds, followed by 120 seconds of dissociation in buffer and surface regeneration. Binding responses were double-referenced by subtraction of mouse control IgG reference surface binding and assay buffer, and the different groups of samples compared

### Assessing Binding or Lack Thereof to A-Beta Monomers

In the primary screen of tissue culture supernatants, A-beta42 monomers and A-beta42 oligomers were used in a direct binding assay. In the secondary screen, A-beta40 monomers and A-beta42 oligomers soluble brain extracts and CSF samples were used in an indirect (capture) binding assay.

### Primary Screen

Tissue culture supernatants were screened for the presence of antibody binding against their cognate cyclic peptide. Each sample was diluted and injected in duplicate over the immobilized peptide and BSA reference surfaces for 120 seconds, followed by injection of running buffer only for a 300-second dissociation phase. After every analytical cycle, the sensor chip surfaces were regenerated. Sensorgrams were double-referenced by subtracting out binding from the BSA reference surfaces and blank running buffer injections, and binding response report points collected in the dissociation phase.

### Oligomer Binding Assay

Next synthetic A-beta 42 oligomers were generated and immobilized as above, antibody binding responses analyzed. Antibody binding responses to A-beta 42 oligomers were compared to binding responses to cyclic.

### Verifying Binding to A-Beta Oligomers.

To further verify and validate A-beta42 Oligomer binding, antibodies were covalently immobilized, followed by the injection over the surface of commercially-prepared stable A-beta42 Oligomers (SynAging SAS, Vandoeuvre-lès-Nancy, France).

### Results

ELISA testing found that the majority of hybridoma clones bound the cyclopeptide. Next clones were tested by ELISA for their binding selectivity for cyclo- and linear-CGGGVVG (SEQ ID NO: 2) compounds. A number of clones preferentially bound cyclo(CGGGVVG)-conjugated-BSA (SEQ ID NO: 2) compared to linear CGGGVVG-conjugated-BSA (SEQ ID NO: 2).

Isotyping revealed that the majority of clones were IgG including IgG1, IgG2a and IgG3 clones. Several IgM and IgA clones were also identified, but not pursued further.

A direct binding analysis using surface plasmon resonance technology was performed to screen for antibodies in tissue culture supernatants that bind to the cyclic peptide of SEQ ID NO: 2.

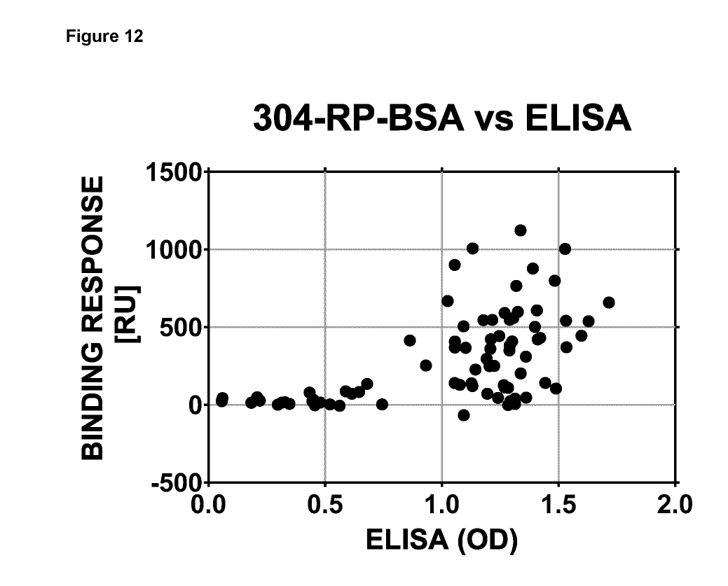

FIG. 12 plots the correlation between the SPR direct binding assay and the ELISA results and shows that there is a correlation between the direct binding and ELISA results.

Clones were retested for their ability to bind cyclic peptide, linear peptide, Abeta 1-42 monomer and Abeta 1-42 oligomers prepared as described above. Binding assays were performed using SPR as described above (Direct binding assays). A number of clones were selected based on the binding assays performed as shown in Table 8.

The selected clones were IgG mAb. Negative numbers are indicative of no binding.

TABLE 8
304

| | Cyclic- | Linear- | A β 42 | A β 42 |
|---|---|---|---|---|
| | Peptide (RU) | Peptide (RU) | Monomer (RU) | Oligomer (RU) |
| 1B5 | 140.1 | −1.6 | −70.2 | 30.8 |
| 1F12 | 430.6 | −19.2 | −37.3 | 31.9 |
| 2G8 | 502 | 97.3 | −18.6 | 57.6 |
| 3B8 | 23.4 | −25.4 | −19.6 | 49.9 |
| 4A8 | 370.8 | −19.6 | −31.9 | 33.2 |
| 4B4 | 422.4 | −14.7 | 26.8 | 55.4 |
| 5F5 | 202 | −18.5 | −51.1 | 51.6 |
| 6E12 | 120.9 | −11.8 | −11.2 | 44.9 |
| 7C5 | 139 | −16.5 | −41.3 | 32.2 |
| 7D7 | 249.6 | −14.8 | −4.7 | 45.9 |
| 7G5 | 13.8 | −18.6 | −7.2 | 61.3 |
| 7H11 | 443.5 | 174.5 | −39.6 | 42 |
| 8E10 | 368.7 | 116.1 | −9.4 | 36.8 |
| 12A11 | 590.8 | 831.4 | 2.8 | 46.3 |
| 12D7 | 367 | −12.3 | −27.7 | 44.5 |
| 12F10 | 1006.5 | 155.4 | 21.2 | 41.8 |

### ELISA Prescreen

The ELISA prescreen of hybridoma supernatants identified clones which showed increased binding to the cyclic peptides compared to the linear peptide. A proportion of the clones were reactive to KLH-epitope linker peptide. These were excluded from further investigation. The majority of the clones were determined to be of the IgG isotype using the isotyping procedure described herein.

### Direct Binding Measured by Surface Plasmon Resonance—Primary Screen

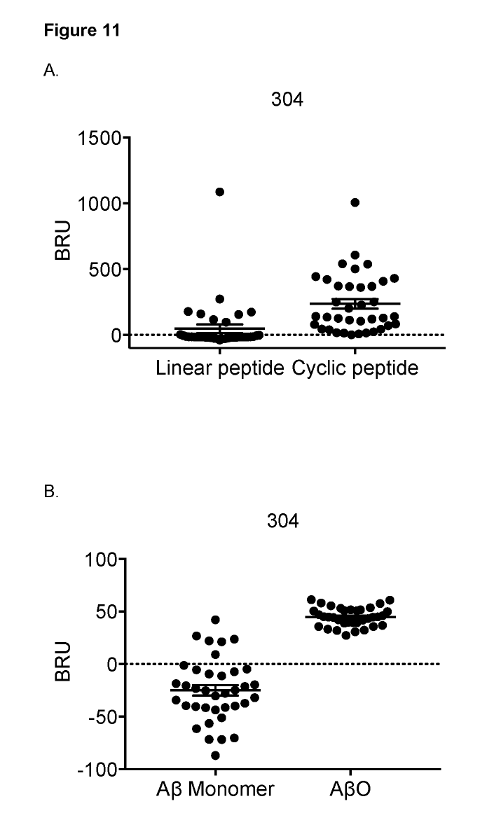

Using surface plasmon resonance the tissue culture supernatants containing antibody clones were tested for direct binding to cyclic peptide, linear peptide (shown in FIG. 11A), A-beta oligomer and A-beta monomer (shown in FIG. 11B). Only IgG clones with no epitope/linker cross reactivity are shown. For most clones binding to linear peptide is at or below zero, in contrast binding to cyclic peptide is positive for the vast majority of clones (FIG. 11A). A similar pattern is seen with A-beta monomer and A-beta oligomer (AβO) binding, all but 6 clones are less reactive to monomer than to the reference surface while all clones bind robustly to AβOs (FIG. 11B).

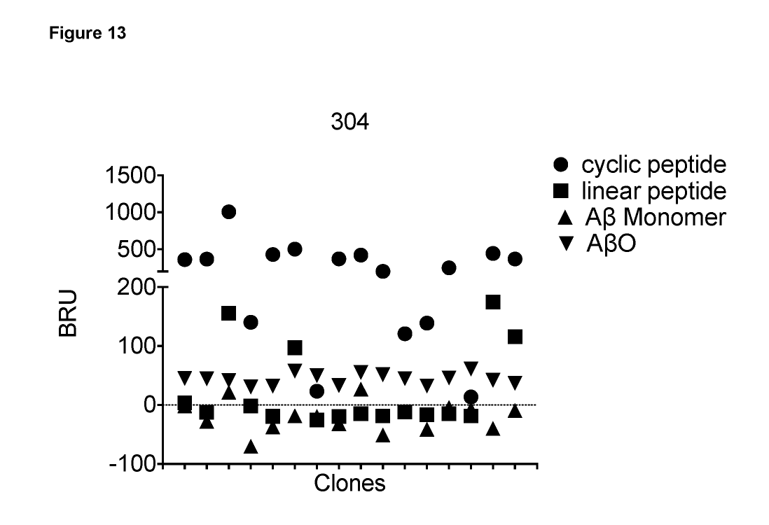

For select clones comparative binding profile is shown in FIG. 13. Each clone is assessed for direct binding using surface plasmon resonance against specific epitope in the context of cyclic peptide, linear peptide, A-beta (Aβ) monomer, and A-beta oligomer (AβO).

### Example 7

### Secondary Screen

### Immunohistochemistry

Immunohistochemistry was performed on frozen human brain sections, with no fixation or antigen retrieval. In a humidified chamber, non-specific staining was blocked by incubation with serum-free protein blocking reagent (Dako Canada Inc., Mississauga, ON, Canada) for 1 h. The following primary antibodies were used for immunostaining: mouse monoclonal isotype controls IgG1, IgG2a, and IgG2b, and anti-amyloidβ 6E10, all purchased from Biolegend, and selected purified clones reactive to the cyclopeptide. All antibodies were used at 1 μg/mL. Sections were incubated at room temperature for 1 h, and washed 3×5 min in TBS-T. Anti-Mouse IgG Horseradish Peroxidase conjugated (1:1000, ECL) was applied to sections and incubated 45 min, then washed 3×5 min in TBS-T. DAB chromogen reagent (Vector Laboratories, Burlington ON, Canada) was applied and sections rinsed with distilled water when the desired level of target to background staining was achieved. Sections were counterstained with Mayer's haematoxylin, dehydrated and cover slips were applied. Slides were examined under a light microscope (Zeiss Axiovert 200M, Carl Zeiss Canada, Toronto ON, Canada) and representative images captured at 20 and 40× magnification using a Leica DC300 digital camera and software (Leica Microsystems Canada Inc., Richmond Hill, ON). Images were optimized in Adobe Photoshop using Levels Auto Correction.

### CSF and Brain Extracts

Human brain tissues were obtained from the University of Maryland Brain and Tissue Bank upon approval from the UBC Clinical Research Ethics Board (C04-0595). CSFs were obtained from patients assessed at the UBC Hospital Clinic for Alzheimer's and Related Disorders. The study was approved by the UBC Clinical Research Ethics Board, and written consent from the participant or legal next of kin was obtained prior to collection of CSF samples. Clinical diagnosis of probable AD was based on NINCDS-ADRDA criteria. CSFs were collected in polypropylene tubes, processed, aliquoted into 100 μL polypropylene vials, and stored at −80° C. within 1 hour after lumbar puncture.

Homogenization: Human brain tissue samples were weighed and subsequently submersed in a volume of fresh, ice cold TBS and EDTA-free protease inhibitor cocktail from Roche Diagnostics (Laval QC, Canada) such that the final concentration of brain tissue was 20% (w/v). Tissue was homogenized in this buffer using a mechanical probe homogenizer (3×30 sec pulses with 30 sec pauses in between, all performed on ice). TBS homogenized samples were then subjected to ultracentrifugation (70,000×g for 90 min). Supernatants were collected, aliquoted and stored at −80° C. The protein concentration of TBS homogenates was determined using a BCA protein assay (Pierce Biotechnology Inc, Rockford Ill., USA).

CSF: CSF was pooled from 9 donors with AD and 9 donors without AD. Samples were analyzed by SPR using purified IgG at a concentration of 30 micrograms/ml for all antibodies. Mouse IgG was used as an antibody control, and all experiments were repeated at least 2 times.

Positive binding in CSF and brain extracts was confirmed using antibody 6E10.

SPR Analysis: 4 brain extracts from AD patients and 4 brain extracts from age-matched controls were pooled and analyzed. Brain samples, homogenized in TBS, included frontal cortex Brodmann area 9. All experiments were performed using a Molecular Affinity Screening System (MASS-1) (Sierra Sensors GmbH, Hamburg, Germany), an analytical biosensor that employs high intensity laser light and high speed optical scanning to monitor binding interactions in real time as described in Example 6. Purified antibodies generated for cyclopeptides described herein were captured on separate flow cells of a protein A-derivatized sensor chip and diluted samples injected over the surfaces for 180 seconds, followed by 120 seconds of dissociation in buffer and surface regeneration. Binding responses were double-referenced by subtraction of mouse control IgG reference surface binding and assay buffer, and the different groups of samples compared.

### Results

### CSF Brain Extracts and Immunohistochemistry

Several clones were tested for their ability to bind A-beta in CSF, soluble brain extracts and tissue samples of cadaveric AD brains are shown in Table 9. Strength of positivity in Table 9 is shown by the number of plus signs.

Table 9 and Table 10 provide data for selected clone's binding selectivity for oligomers over monomer measured as described herein by SPR.

IHC results are also summarized in Table 9 where “+/−” denotes staining similar to or distinct from isotype control but without clear plaque morphology.

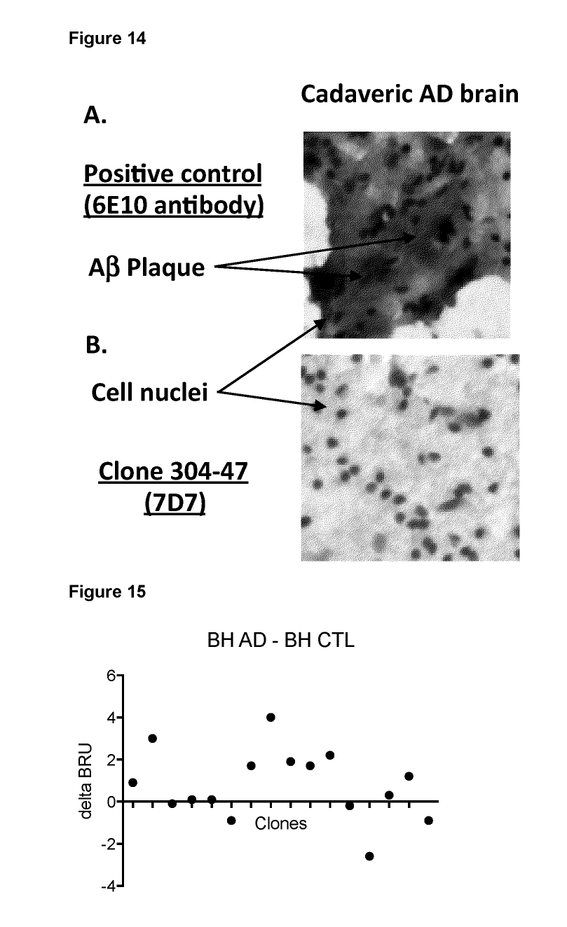

FIG. 14 shows an example of the lack of plaque staining on fresh frozen sections with clone 304-47 (7D7) (B) compared to the positive plaque staining seen with 6E10 antibody (A).

FIG. 15 shows antibodies raised to the cyclopeptide comprising GGVV (SEQ ID NO: 1) included antibodies that bound A-beta in brain extracts of AD patients to a greater extent than those of control patients.

As shown in Tables 9, 10 and FIG. 14, using a cyclopeptide comprising GGVV (SEQ ID NO: 1) as an immunogen, produced antibody clones that bound to A-beta in brain extracts and/or CSF, but did not appreciably bind to monomers on SPR, and did not appreciably bind to plaque fibrils by IHC.

TABLE 9
Table 9: Summary of binding characteristics

| | | Oligomers/ | CSF | Brain Extract | IHC - Plaque |
|---|---|---|---|---|---|
| | Clone # | Monomers | AD/Non-AD | AD/Non-AD | Staining |
| cyclo(CGGGVVG) | 38 (1B5) | ++ | − | + | − |
| (SEQ ID NO: 2) | 41 (3B8) | ++ | ++ | + | +/− |
| | 47 (7D7) | + | + | + | − |

* Scoring is relative to other clones in the same sample category.

TABLE 10
A-beta Oligomer binding RU values
subtracted for monomer binding

| Clone tested | 304-41 |
|---|---|
| RU | 6.4 |

### Example 8

### Synthetic Oligomer Binding

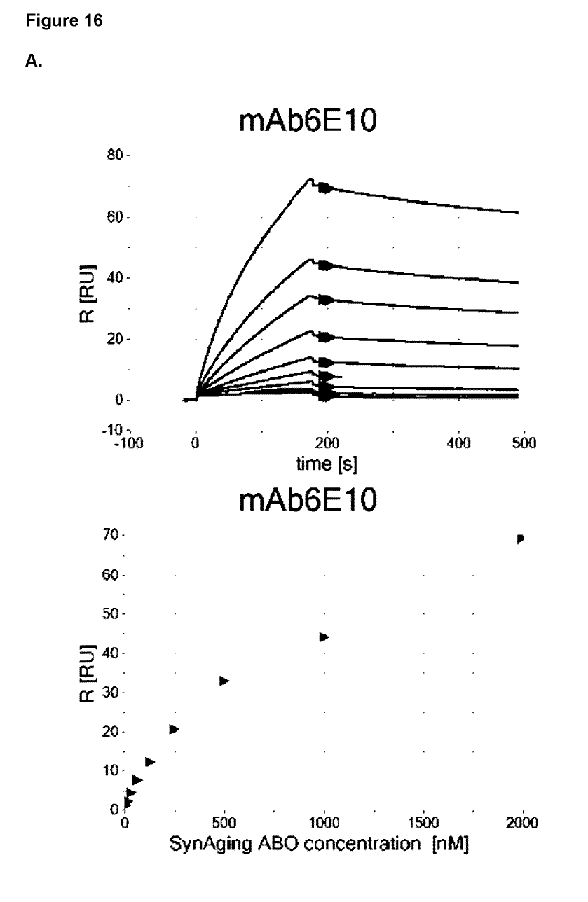

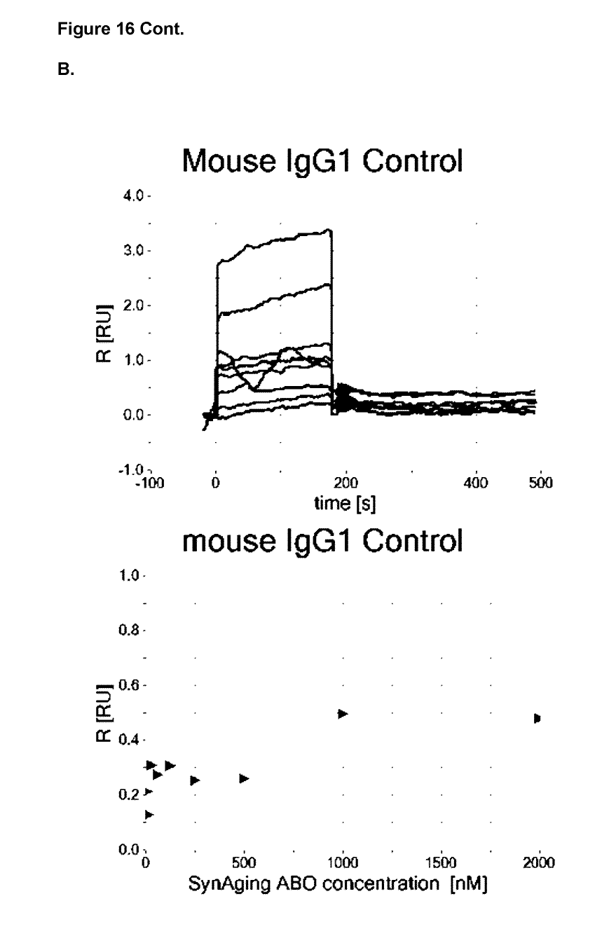

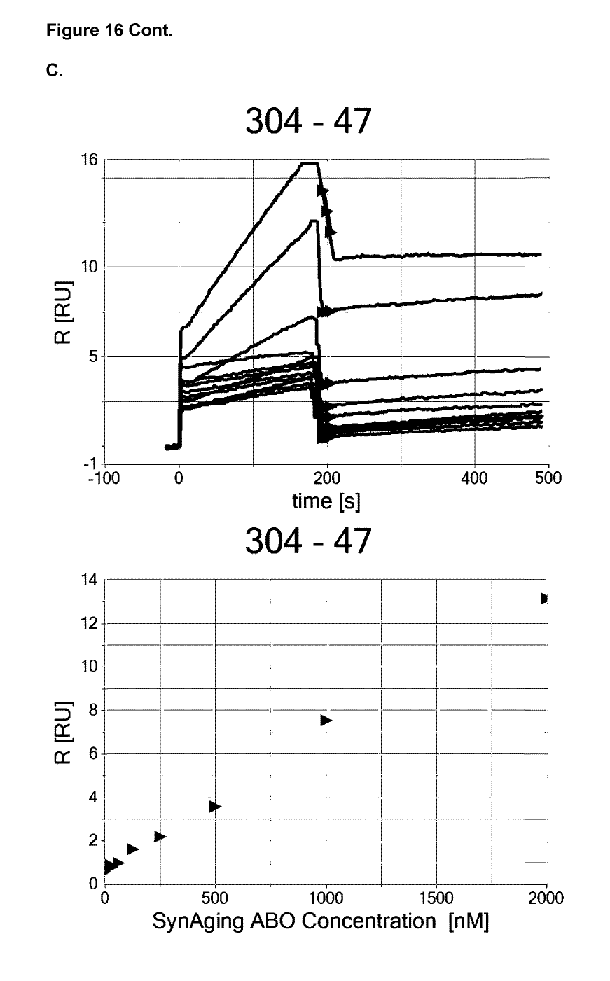

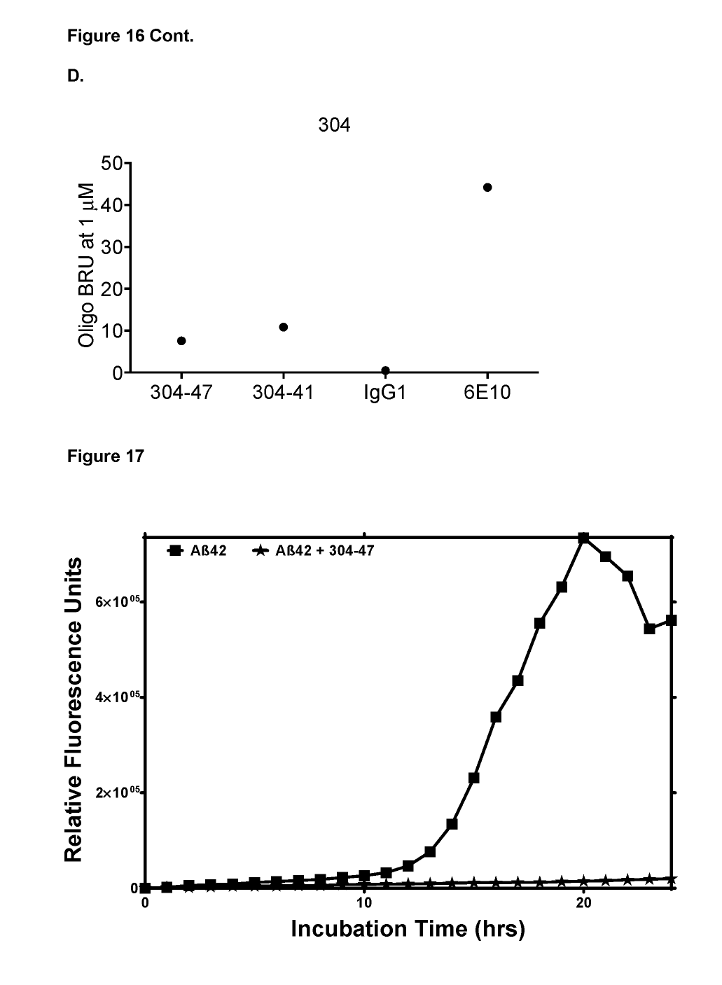

Serial 2-fold dilutions (7.8 nM to 2000 nM) of commercially-prepared synthetic amyloid beta oligomers (SynAging SAS, Vandoeuvre-lès-Nancy, were tested for binding to covalently immobilized antibodies. Results for control antibody mAb6E10 is shown in FIG. 16A and mouse control IgG is shown in FIG. 16B. FIG. 16C shows results using an antibody raised against cyclo(CGGGVVG) (SEQ ID NO: 2).

### Example 9

### Immunohistochemistry on Formalin Fixed Tissues

Human brain tissue was assessed using antibodies raised to cyclo CGGGVVG (SEQ ID NO: 2. The patient had been previously characterized and diagnosed with Alzheimer's disease with a tripartite approach: (i) Bielschowsky silver method to demonstrate senile plaques and neurofibrillary tangles, (ii) Congo red to demonstrate amyloid and (iii) tau immunohistochemistry to demonstrate tangles and to confirm the senile plaques are “neuritic”. This tissue was used to test plaque reactivity of selected monoclonal antibody clones. The brain tissues were fixed in 10% buffered formalin for several days and paraffin processed in the Sakura VIP tissue processors. Tissue sections were probed with 1 μg/ml of antibody with and without microwave antigen retrieval (AR). The pan-amyloid beta reactive antibody 6E10 was included along with selected antibody clones as a positive control. Antibodies were diluted in Antibody Diluent (Ventana), color was developed with OptiView DAB (Ventana). The staining was performed on the Ventana Benchmark XT IHC stainer. Images were obtained with an Olympus BX45 microscope. Images were analyzed blind by a professional pathologist with expertise in neuropathology.

As shown in Table 11 below, using fixed tissue, the tested antibodies were negative for specific staining of senile plaque amyloid with or without antigen retrieval. 6E10 was used as the positive control.

TABLE 11

| | | Convincing evidence of specific | |
|---|---|---|---|
| | | staining of senile plaque amyloid | |
| Epitope | Antibodies to test | Without AR | Plus AR |
| 304 | 41 | Neg | Neg |
| | 45 | Neg | Neg |
| | 47 | Neg | Neg |
| | 52 (12D7) | Neg | Neg |
| Positive Control | 6E10 | Strongly | Strongly |
| | | positive | positive |

### Example 10

### Inhibition of Oligomer Propagation

The biological functionality of antibodies was tested in vitro by examining their effects on Amyloid Beta (Aβ) aggregation using the Thioflavin T (ThT) binding assay. Aβ aggregation is induced by and propagated through nuclei of preformed small Aβ oligomers, and the complete process from monomeric Aβ to soluble oligomers to insoluble fibrils is accompanied by concomitantly increasing beta sheet formation. This can be monitored by ThT, a benzothiazole salt, whose excitation and emission maxima shifts from 385 to 450 nm and from 445 to 482 nm respectively when bound to beta sheet-rich structures and resulting in increased fluorescence Briefly, Aβ 1-42 (Bachem Americas Inc., Torrance, Calif.) was solubilized, sonicated, diluted in Tris-EDTA buffer (pH7.4) and added to wells of a black 96-well microtitre plate (Greiner Bio-One, Monroe, N.C.) to which equal volumes of cyclopeptide raised antibody or irrelevant mouse IgG antibody isotype controls were added, resulting in a 1:5 molar ratio of Aβ1-42 peptide to antibody. ThT was added and plates incubated at room temperature for 24 hours, with ThT fluorescence measurements (excitation at 440 nm, emission at 486 nm) recorded every hour using a Wallac Victor3v 1420 Multilabel Counter (PerkinElmer, Waltham, Mass.). Fluorescent readings from background buffer were subtracted from all wells, and readings from antibody only wells were further subtracted from the corresponding wells.

As shown in FIG. 17, Aβ42 aggregation, as monitored by ThT fluorescence, demonstrated a sigmoidal shape characterized by an initial lag phase with minimal fluorescence, an exponential phase with a rapid increase in fluorescence and finally a plateau phase during which the Aβ molecular species are at equilibrium and during which there is no increase in fluorescence. Co-incubation of Aβ42 with an irrelevant mouse antibody did not have any significant effect on the aggregation process. In contrast, co-incubation of Aβ42 with the test antibodies completely inhibited all phases of the aggregation process. Results obtained with antibody clone 47 (7D7; IgG1 isotype) are shown in FIG. 17. As the ThT aggregation assay mimics the in vivo biophysical/biochemical stages of Aβ propagation and aggregation from monomers, oligomers, protofibrils and fibrils that is pivotal in AD pathogenesis, the antibodies raised to cyclo CGGGVVG (SEQ ID NO: 2) demonstrate the potential to completely abrogate this process. Isotype control performed using mouse IgG control showed no inhibition.

### Example 11

Achieving the optimal profile for Alzheimer's immunotherapy: Rational generation of antibodies specific for toxic A-beta oligomers

Objective: Generate antibodies specific for toxic amyloid-β oligomers (AβO)

Background: Current evidence suggests that propagating prion-like strains of AβO, as opposed to monomers and fibrils, are preferentially toxic to neurons and trigger tau pathology in Alzheimer's disease (AD). In addition, dose-limiting adverse effects have been associated with Aβ fibril recognition in clinical trials. These observations suggest that specific neutralization of toxic AβOs may be desirable for safety and efficacy.

Design/Methods: Computational simulations were employed as described herein, using molecular dynamics with standardized force-fields to perturb atomic-level structures of Aβ fibrils deposited in the Protein Data Base. It was hypothesized that weakly-stable regions are likely to be exposed in nascent protofibrils or oligomers. Clustering analysis, curvature, exposure to solvent, solubility, dihedral angle distribution, and Ramachandran angle distributions were all used to characterize the conformational properties of predicted epitopes, which quantify differences in the antigenic profile when presented in the context of the oligomer vs the monomer or fibril. The candidate peptide epitopes were synthesized in a cyclic format that may mimic regional AβO conformation, conjugated to a carrier protein, and used to generate monoclonal antibodies in mice. Purified antibodies were screened by SPR and immunohistochemistry.

### Results:

Sixty-six IgG clones against 5 predicted epitopes were selected for purification based on their ability to recognize the cognate structured peptide and synthetic AβO, with little or no binding to unstructured peptide, linker peptide, or Aβ monomers. Additional screening identified antibodies that preferentially bound to native soluble AβO in CSF and brain extracts of AD patients compared to controls. Immunohistochemical analysis of AD brain allowed for selection of antibody clones that do not react with plaque.

Conclusion: Computationally identified AβO epitopes allowed for the generation of antibodies with the desired target profile of selective binding to native AD AβOs with no significant cross-reactivity to monomers or fibrils.

### Example 12

### Toxicity Inhibition Assay

The inhibition of toxicity of A-beta42 oligomers by antibodies raised to the cyclopeptide can be tested in a rat primary cortical neuron assay.

Antibody and control IgG are each adjusted to a concentration such as 2 mg/mL. Various molar ratios of A-beta oligomer and antibody are tested along with a vehicle control, A-beta oligomer alone and a positive control such as the neuroprotective peptide humanin HNG.

An exemplary set up is shown in Table 12.

Following preincubation for 10 minutes at room temperature, the volume is adjusted to 840 microlitres with culture medium. The solution is incubated for 5 min at 37 C. The solution is then added directly to the primary cortical neurons and cells are incubated for 24 h. Cell viability can be determined using the MTT assay.

TABLE 12

| AβO/AB molar | AβO | AβO | AB | AB | Medium | Final volume |
|---|---|---|---|---|---|---|
| ratio | (μL) | (μM) | (μM) | (μL) | (μL) | (μL) |
| 5/1 | 1.68 | 4.2 | 0.84 | 12.73 | 185.6 | 200 |
| 1/1 | 1.68 | 4.2 | 4.20 | 63.64 | 134.7 | 200 |
| 1/2 | 1.68 | 4.2 | 8.4 | 127.27 | 71.1 | 200 |

AβO working solution: 2.2 mg/mL - 500 μM
CTRL vehicle: 1.68 μL of oligomer buffer + 127.3 μL PBS + 711 μL culture medium
CTRL AβO: 1.68 μL of AβO + 127.3 μL PBS + 711 μL culture medium
CTRL HNG: 1.68 μL of AβO + 8.4 μL HNG (100 nM final) + 127.3 μL PBS + 702.6 μL culture medium

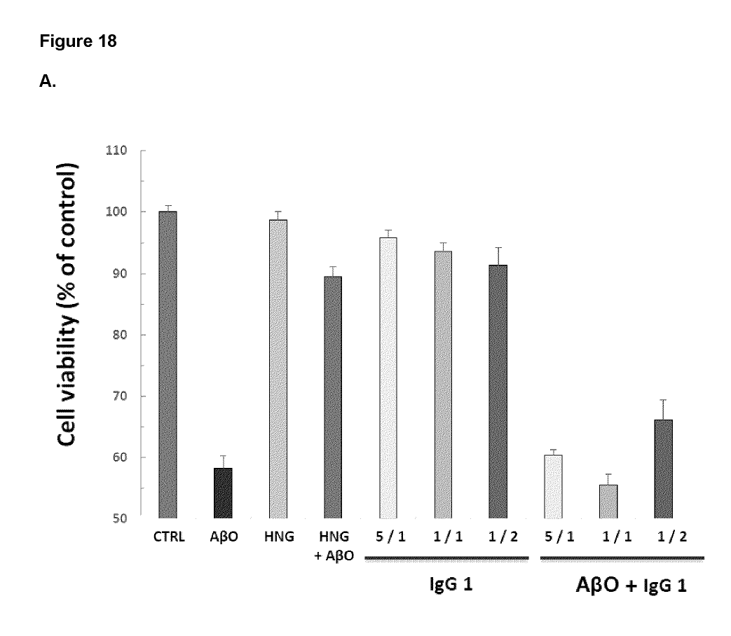

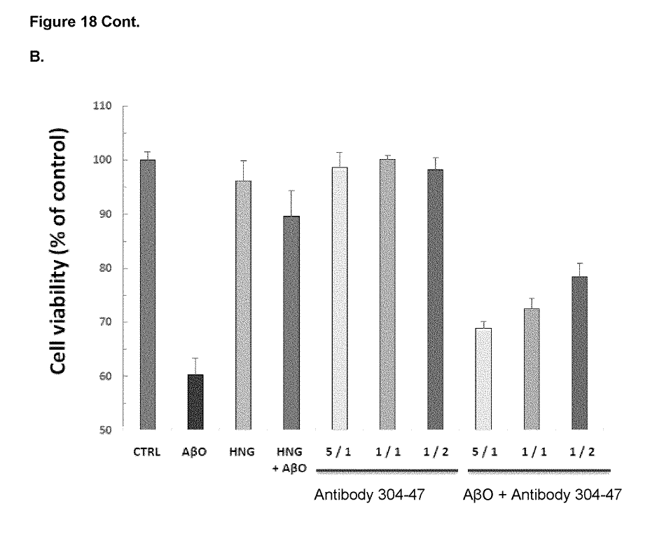

This test was conducted using 304 antibody clone 47 which demonstrated inhibition of A-beta oligomer toxicity. (FIG. 18).

### Example 13

### In Vivo Toxicity Inhibition Assay

The inhibition of toxicity of A-beta42 oligomers by antibodies raised to the cyclopeptide can be tested in vivo in mouse behavioral assays.

The antibody and an isotype control are each pre-mixed with A-beta42 oligomers at 2 or more different molar ratios prior to intracerebroventricular (ICV) injection into mice. Control groups include mice injected with vehicle alone, oligomers alone, antibody alone, and a positive control such as the neuroprotective peptide humanin. Alternatively, the antibodies can be administered systemically prior to, during, and/or after ICV injection of the oligomers. Starting approximately 4-7 days post ICV injection of oligomers, cognition is assessed in behavioral assays of learning and memory such as the mouse spatial recognition test (SRT), Y-Maze assay, Morris water maze model and novel object recognition model (NOR).

The mouse spatial recognition test (SRT) assesses topographical memory, a measure of hippocampal function (SynAging). The model uses a two-chamber apparatus, in which the chambers differ in shape, pattern and color (i.e. topographical difference). The chambers are connected by a clear Plexiglass corridor. Individual mice are first placed in the apparatus for a 5 min exploration phase where access to only one of the chambers is allowed. Mice are then returned to their home cage for 30 min and are placed back in the apparatus for a 5 min “choice” phase during which they have access to both chambers. Mice with normal cognitive function remember the previously explored chamber and spend more time in the novel chamber. A discrimination index (DI) is calculated as follows: DI=(TN−TF)/(TN+TF), in which TN is the amount of time spent in the novel chamber and TF is the amount of time spent in the familiar chamber. Toxic A-beta oligomers cause a decrease in DI which can be partially rescued by the humanin positive control. Performance of this assay at different time points post ICV injection can be used to evaluate the potential of antibodies raised to the cyclopeptide to inhibit A-beta oligomer toxicity in vivo.

The Y-maze assay (SynAging) is a test of spatial working memory which is mainly mediated by the prefrontal cortex (working memory) and the hippocampus (spatial component). Mice are placed in a Y-shaped maze where they can explore 2 arms. Mice with intact short-term memory will alternate between the 2 arms in successive trials. Mice injected ICV with toxic A-beta oligomers are cognitively impaired and show random behavior with alternation close to a random value of 50% (versus ˜70% in normal animals). This impairment is partially or completely reversed by the cholinesterase inhibitor donepezil (Aricept) or humanin, respectively. This assay provides another in vivo assessment of the protective activity of test antibodies against A-beta oligomer toxicity.

The Morris water maze is another widely accepted cognition model, investigating spatial learning and long-term topographical memory, largely dependent on hippocampal function (SynAging). Mice are trained to find a platform hidden under an opaque water surface in multiple trials. Their learning performance in recalling the platform location is based on visual clues and video recorded. Their learning speed, which is the steadily reduced time from their release into the water until finding the platform, is measured over multiple days. Cognitively normal mice require less and less time to find the platform on successive days (learning). For analyzing long-term memory, the test is repeated multiple days after training: the platform is taken away and the number of crossings over the former platform location, or the time of the first crossing, are used as measures to evaluate long-term memory. Mice injected ICV with toxic A-beta oligomers show deficits in both learning and long-term memory and provide a model for evaluating the protective activity of test antibodies.

The Novel Object Recognition (NOR) model utilizes the normal behavior of rodents to investigate novel objects for a significantly longer time than known objects, largely dependent on perirhinal cortex function (SynAging). Mice or rats are allowed to explore two identical objects in the acquisition trial. Following a short inter-trial interval, one of the objects is replaced by a novel object. The animals are returned to the arena and the time spent actively exploring each object is recorded. Normal rodents recall the familiar object and will spend significantly more time exploring the novel object. In contrast, A-beta oligomer-treated rodents exhibit clear cognitive impairment and will spend a similar amount of time investigating both the ‘familiar’ and ‘novel’ object. This can be transiently reversed with known clinical cognitive enhancers (e.g. donepezil). The NOR assay can be performed multiple times in longitudinal studies to assess the potential cognitive benefit of test antibodies.

In addition to behavioral assays, brain tissue can be collected and analyzed for levels of synaptic markers (PSD95, SNAP25, synaptophysin) and inflammation markers (IL-1-beta). Mice are sacrificed at ˜14 days post-ICV injection of oligomers and perfused with saline. Hippocampi are collected, snap frozen and stored at −80° C. until analyzed. Protein concentrations of homogenized samples are determined by BCA. Concentration of synaptic markers are determined using ELISA kits (Cloud-Clone Corp, USA). Typically, synaptic markers are reduced by 25-30% in mice injected with A-beta oligomers and restored to 90-100% by the humanin positive control. Concentrations of the IL-1-beta inflammatory markers are increased approximately 3-fold in mice injected with A-beta oligomers and this increase is largely prevented by humanin. These assays provide another measure of the protective activity of test antibodies at the molecular level.

### Example 14

### In Vivo Propagation Inhibition Assay

In vivo propagation of A-Beta toxic oligomers and associated pathology can be studied in various rodent models of Alzheimer's disease (AD). For example, mice transgenic for human APP (e.g. APP23 mice) or human APP and PSEN1 (APPPS1 mice) express elevated levels of A-beta and exhibit gradual amyloid deposition with age accompanied by inflammation and neuronal damage. Intracerebral inoculation of oligomer-containing brain extracts can significantly accelerate this process13, 14). These models provide a system to study inhibition of A-beta oligomer propagation by test antibodies administered intracerebrally or systemically.

### Example 15

### CDR Sequencing

304-7D7.1 which was determined to have an IgG1 heavy chain and a kappa light chain was selected for CDR and variable regions of the heavy and light chains.

RT-PCR was carried out using 5′ RACE and gene specific reverse primers which amplify the appropriate mouse immunoglobulin heavy chain (IgG1/IgG3/IgG2A) and light chain (kappa) variable region sequences.

The specific bands were excised and cloned into pCR-Blunt II-TOPO vector for sequencing, and the constructs were transformed into E. coli

At least 8 colonies of each chain were picked & PCR screened for the presence of amplified regions prior to sequencing. Selected PCR positive clones were sequenced.

The CDR sequences are in Table 13. The consensus DNA sequence and protein sequences of the variable portion of the heavy and light chain are provided in Table 14.

TABLE 13

| | | | SEQ |
|---|---|---|---|
| | | | ID |
| Chain | CDR | Sequence | NO. |
| Heavy | CDR-H1 | GFTFSNYW | 17 |
| | CDR-H2 | IRLKSYNYAT | 18 |
| | CDR-H3 | LRWIDY | 19 |
| Light | CDR-L1 | QDINSY | 20 |
| | CDR-L2 | RAN | 21 |
| | CDR-L3 | PQYDEFPYT | 22 |

TABLE 14
Consensus DNA sequence and translated protein sequences of the
variable region. The complementarity determining regions (CDRs)
are underlined according to IMTG/LIGM-DB.

| Isotype | Consensus DNA Sequence | Protein sequence |
|---|---|---|
| IgG1 | ATGTATTTGGGACTGAACTGTGTATTCATAGTTTTTCTCTTAAAA | MYLGLNCVFIVFLLKG |
| SEQ ID | GGTGTCCAGAGTGAAGTGAAGCTTGAGGAGTCTGGAGGAGGCTTG | VQSEVKLEESGGGLVQ |
| NO: | GTGCAACCTGGAGGATCCATGAAACTCTCCTGTGTTGCCTCTGGA | PGGSMKLSCVASGFTF |
| 23, 24 | TTCACTTTCAGTAACTACTGGATGAACTGGGTCCGCCAGTCTCCA | SNYWMNWVRQSPEKGL |
| | GAGAAGGGGCTTGAGTGGGTTGCTGAAATTAGATTGAAATCTTAT | EWVAEIRLKSYNYATH |
| | AATTATGCAACACATTATGCGGAGTCTGTGAAAGGGAGGTTCACC | YAESVKGRFTISRDDS |
| | ATCTCAAGAGATGATTCCAAAAGTAGTGTCTACCTGCAAATGAAC | KSSVYLQMNNLRAEDT |
| | AACTTAAGAGCTGAAGACACTGGCATTTATTACTGTTTACGGTGG | GIYYCLRWIDYWGQGT |
| | ATCGACTACTGGGGCCAAGGCACCACTCTCACAGTCTCCTCAGCC | TLTVSSAKTT |
| | AAAACGACA | |
| Kappa | ATGGACATGAGGACCCCTGCTCAGTTTCTTGGAATCTTGTTGCTC | MDMRTPAQFLGILLLW |
| SEQ ID | TGGTTTCCAGGTATCAAATGTGACATCAAGATGACCCAGTCTCCA | FPGIKCDIKMTQSPSS |
| NO: | TCTTCCATGTATGCATCTCTAGGAGAGAGAGTCACTATCACTTGC | MYASLGERVTITCKAS |
| 25, 26 | AAGGCGAGTCAGGACATTAATAGCTATTTAAGCTGGTTCCAGCAG | QDINSYLSWFQQKPGK |
| | AAACCAGGGAAATCTCCTAAGACCCTGATCTATCGTGCAAACAGA | SPKTLIYRANRLVDGV |
| | TTGGTAGATGGGGTCCCATCAAGGTTCAGTGGCAGTGGATCTGGG | PSRFSGSGSGQDYSLT |
| | CAAGATTATTCTCTCACCATCAGCAGCCTGGAGTATGAAGATATG | ISSLEYEDMGIYYCPQ |
| | GGAATTTATTATTGTCCACAGTATGATGAGTTTCCGTACACGTTC | YDEFPYTFGGGTKLEI |
| | GGAGGGGGGACCAAGCTGGAAATAAAACGGGCTGATGCT | KRADA |

TABLE 15
A-beta epitope Sequences and A-beta
sequences with linker

| | |
|---|---|
| GGVV | (SEQ ID NO: 1) |
| CGGGVVG, cyclo (CGGGVVG) | (SEQ ID NO: 2) |
| CGGVVG, C-PEG2-GGVVG | (SEQ ID NO: 3) |
| CGGGVV, CGGGVV-PEG2 | (SEQ ID NO: 4) |
| VGGV | (SEQ ID NO: 5) |
| VGGVV | (SEQ ID NO: 6) |
| VGGVVI | (SEQ ID NO: 7) |
| GGVVI | (SEQ ID NO: 8) |
| GGGVVG | (SEQ ID NO: 9) |
| GGVVG | (SEQ ID NO: 10) |
| GGGVV | (SEQ ID NO: 11) |
| CGGGVVGC | (SEQ ID NO: 12) |
| AIIGLMVGGVV | (SEQ ID NO: 13) |
| MVGGVV | (SEQ ID NO: 14) |
| GGVVIA | (SEQ ID NO: 15) |

TABLE 16
DAEFRHDSGYEVHHQKLVFFAEDVGSNKGAIIGLMVGGVVIA
(SEQ ID NO: 16)

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the preferred embodiments and examples, but should be given the broadest interpretation consistent with the description as a whole.

### CITATIONS FOR REFERENCES REFERRED TO IN THE SPECIFICATION

[1] Gabriela A. N. Cresol, Stefan J. Hermans, Michael W. Parker, and Luke A. Miles. Molecular basis for mid-region amyloid-b capture by leading Alzheimer's disease immunotherapies SCIENTIFIC REPORTS|5: 9649, 2015|DOI: 10.1038/srep09649
[2] Vincent J. Hilser and Ernesto Freire. Structure-based calculation of the equilibrium folding pathway of proteins, correlation with hydrogen exchange protection factors. J. Mol. Biol., 262:756-772, 1996. The COREX approach.
[3] Samuel I. A. Cohen, Sara Linse, Leila M. Luheshi, Erik Hellstrand, Duncan A. White, Luke Rajah, Daniel E. Otzen, Michele Vendruscolo, Christopher M. Dobson, and Tuomas P. J. Knowles. Proliferation of amyloid-β42 aggregates occurs through a secondary nucleation mechanism. Proc. Natl.l Acad. Sci. USA, 110(24):9758-9763, 2013.
[4] Pietro Sormanni, Francesco A. Aprile, and Michele Vendruscolo. The camsol method of rational design of protein mutants with enhanced solubility. Journal of Molecular Biology, 427(2):478-490, 2015.
[5] Deborah Blacker, MD, ScD; Marilyn S. Albert, PhD; Susan S. Bassett, PhD; Rodney C. P. Go, PhD; Lindy E. Harrell, MD, PhD; Marshai F. Folstein, MD Reliability and Validity of NINCDS-ADRDA Criteria for Alzheimer's Disease The National Institute of Mental Health Genetics Initiative. Arch Neurol. 1994; 51(12):1198-1204. doi:10.1001/archneur.1994.00540240042014.
[6] Hamley, I. W. PEG-Peptide Conjugates 2014; 15, 1543-1559; dx.doi.org/10.1021/bm500246w
[7 ] Roberts, M J et al Chemistry for peptide and protein PEGylation 64: 116-127.
[8] J. X. Lu, W. Qiang, W. M. Yau, C. D. Schwieters, S. C. Meredith, R. Tycko, MOLECULAR STRUCTURE OF BETA-AMYLOID FIBRILS IN ALZHEIMER'S DISEASE BRAIN TISSUE. CELL Vol. 154 p. 1257 (2013)
[9] Y. Xiao, B. MA, D. McElheny, S. Parthasarathy, F. Long, M. Hoshi, R. Nussinov, Y. Ishii, A BETA (1-42) FIBRIL STRUCTURE ILLUMINATES SELF-RECOGNITION AND REPLICATION OF AMYLOID IN ALZHEIMER'S DISEASE. NAT. STRUCT. MOL. BIOL. Vol. 22 p. 499 (2015).
[10] A. Petkova, W. Yau, R. Tycko EXPERIMENTAL CONSTRAINTS ON QUATERNARY STRUCTURE IN ALZHEIMER'S BETA-AMYLOID FIBRILS BIOCHEMISTRY V. 45 498 2006.
[11] Paganetti P A, Lis M, Klafi H W, Staufenbiel M. Amyloid precursor protein truncated at any of the γ-secretase sites is not cleaved to β-amyloid, J. Neurosci. Res. 46 (1996) 283-293.
[12] Weihofen A, Lemberg M K, Friedmann E, Rueeger H, Schmitz A, Pagnetti P, Rovelli G, Martoglio B. Targeting presenillin-type aspartic protease signal peptide peptidase with γ-secretase inhibitors. J Biol Chem. 2003; 278(19),16528-33.
[13] Kahlert H, Weber B, Teppke M, Wahl R, Cromwell O, Fiebig H. Characterization of major allergens of Parietaria officinalis. Int Arch Allergy Immunol 1996 February; 109(2):141-9.

1. A cyclic compound comprising: an A-beta peptide where the A-beta peptide comprises GVV and up to 6 A-beta contiguous residues, and a linker, wherein the linker is covalently coupled to the A-beta peptide N-terminus residue and the A-beta peptide C-terminus residue.

2. The cyclic compound of claim 1, wherein the peptide is selected from GGVV (SEQ ID NO:1), GGVVI (SEQ ID NO:8), VGGVVI (SEQ ID NO:7), VGGVV (SEQ ID NO:6), and VGGV (SEQ ID NO:5).

3.-5. (canceled)

6. The cyclic compound of claim 1, wherein the cyclic compound further comprises a detectable label and/or wherein the linker comprises or consists of amino acids GCG or a PEG molecule.

7.-11. (canceled)

12. An immunogen comprising the cyclic compound of claim 1, wherein the compound is coupled to a carrier protein or an immunogenicity enhancing agent, optionally wherein the carrier protein is bovine serum albumin (BSA) or the immunogenicity-enhancing agent is keyhole Keyhole Limpet Haemocyanin (KLH).

13.-14. (canceled)

15. A composition comprising the compound of claim 1 or an immunogen comprising said compound coupled to a carrier protein or an immunogenicity enhancing agent.

16. The composition of claim 15, further comprising an adjuvant, optionally wherein the adjuvant is aluminum phosphate or aluminum hydroxide.

17. (canceled)

18. An isolated antibody that specifically binds to an A-beta peptide having a sequence of GGVV (SEQ ID NO:1) or a related epitope sequence presented in a cyclic compound, optionally as set forth in any one of SEQ ID NOS: 1-15.

19.-21. (canceled)

22. The antibody of claim 18, wherein the antibody selectively binds to a cyclic compound comprising GGVV (SEQ ID NO:1) over a corresponding linear peptide, or A-beta oligomer over A-beta monomer and/or A-beta fibril.

23. The antibody of claim 18, wherein the antibody is at least 2 fold more selective for the cyclic compound over the corresponding linear peptide, or more selective for A-beta oligomer over A-beta monomer and/or A-beta fibril.

24.-27. (canceled)

28. The antibody of claim 18, wherein the antibody is a monoclonal antibody or a polyclonal antibody, or wherein the antibody is a humanized antibody.

29. (canceled)

30. The antibody of claim 18, therein the antibody is an antibody binding fragment selected from Fab, Fab′, F(ab′)2, scFv, dsFv, ds-scFv, dimers, nanobodies, minibodies, diabodies, and multimers thereof.

31. The antibody of claim 18, comprising a light chain variable region and a heavy chain variable region, optionally fused, the heavy chain variable region comprising complementarity determining regions CDR-H1, CDR-H2 and CDR-H3, the light chain variable region comprising complementarity determining region CDR-L1, CDR-L2 and CDR-L3 and with the amino acid sequences of said CDRs comprising the sequences:

| | | |
|---|---|---|
| CDR-H1 | GFTFSNYW | (SEQ ID NO: 17) |
| CDR-H2 | IRLKSYNYAT | (SEQ ID NO: 18) |
| CDR-H3 | LRWIDY | (SEQ ID NO: 19) |
| CDR-L1 | QDINSY | (SEQ ID NO: 20) |
| CDR-L2 | RAN | (SEQ ID NO: 21) |
| CDR-L3 | PQYDEFPYT. | (SEQ ID NO: 22) |

32. The antibody of claim 18, wherein the antibody comprises a heavy chain variable region comprising: i) an amino acid sequence as set forth in SEQ ID NO: 24; ii) an amino acid sequence with at least 50%, at least 60%, at least 70%, at least 80% sequence identity to SEQ ID NO: 24, wherein the CDR sequences are as set forth in SEQ ID NO: 17, 18 and 19, or iii) a conservatively substituted amino acid sequence i), and/or wherein the antibody comprises a light chain variable region comprising i) an amino acid sequence as set forth in SEQ ID NO: 26, ii) an amino acid sequence with at least 50%, at least 60%, at least 70%, at least 80% 70% sequence identity to SEQ ID NO: 26, wherein the CDR sequences are as set forth in SEQ ID NO: 20, 21 and 22, or iii) a conservatively substituted amino acid sequence of i), optionaly wherein the heavy chain variable region amino acid sequence is encoded by a nucleotide sequence as set forth in SEQ ID NO: 23 or a codon degenerate or optimized version thereof; and/or the antibody comprises a light chain variable region amino acid sequence encoded by a nucleotide sequence as set out in SEQ ID NO: 25 or a codon degenerate or optimized version thereof.

33.-35. (canceled)

36. The antibody of claim 18, wherein the antibody competes for binding to human A-beta with an antibody comprising the CDR sequences as recited in Table 13.

37. An immunoconjugate comprising the antibody of claim 18 and a detectable label or cytotoxic agent, optionally wherein the detectable label comprises a positron emitting radionuclide, optionally for use in subject imaging such as PET imaging.

38. (canceled)

39. A composition comprising the compound of claim 1, immunogen comprising said compound, an antibody that selectively binds said compound or immunogen, or an immunoconjugate comprising said antibody, optionally with a diluent.

40. A nucleic acid molecule encoding a proteinaceous portion of the compound of claim 1, or an antibody that binds said compound, optionally comprised in a vector.

41. (canceled)

42. A cell expressing the antibody of claim 18.

43. A kit comprising the compound of claim 1, an immunogen, comprising said compound, an antibody, or immunoconjugate that specifically or selectively binds to said compound or immunogen, a nucleic acid molecule encoding a proteinaceous portion of the compound, a vector comprising said nucleic acid molecule, or a cell expressing said antibody.

44. A method of making the antibody of claim 18, comprising administering a compound of claim 1, or an immunogen comprising said compound, or a composition comprising the compound or immunogen to a subject and isolating antibody and/or cells expressing antibody specific and/or selective for the compound or immunogen administered, and/or A-beta oligomers, optionally lacking or having negligible binding to a linear peptide comprising the A-beta peptide and/or lacking or having negligible plaque binding.

45. A method of determining if a biological sample contains A-beta, the method comprising:
a. contacting the biological sample with the antibody of claim 18 or an immunoconjugate comprising said antibody under conditions permissive for forming an antibody:A-beta oligomer complex; and
b. detecting the presence of any complex.

46.-51. (canceled)

52. A method of measuring a level of A-beta in a subject, the method comprising:
a. administering to a subject at risk or suspected of having or having AD, an immunoconjugate of claim 37 wherein the antibody is conjugated to a detectable label; and
b. detecting the label, optionally quantitatively detecting the label, optionally wherein the label is a positron emitting radionuclide.

53. (canceled)

54. A method of inducing an immune response in a subject, comprising administering to the subject a compound or combination of compounds of claim 1, optionally a cyclic compound comprising GGVV (SEQ ID NO:1) or a related epitope peptide sequence, an immunogen and/or composition comprising said compound or said immunogen; and optionally isolating cells and/or antibodies that specifically or selectively bind the A-beta peptide in the compound or immunogen administered.

55. A method of inhibiting A-beta oligomer propagation, the method comprising contacting a cell or tissue expressing A-beta with or administering to a subject in need thereof an effective amount of an A-beta oligomer specific and/or selective antibody of claim 19, or an immunoconjugate comprising said antibody to inhibit A-beta aggregation and/or oligomer propagation.

56. A method of treating AD and/or other A-beta amyloid related diseases, the method comprising administering to a subject in need thereof 1) an effective amount of an antibody of claim 18, optionally an A-beta oligomer specific and/or selective antibody, or a pharmaceutical composition comprising said antibody; 2) administering an isolated cyclic compound comprising GGVV (SEQ ID NO:1) or a related epitope sequence or immunogen or pharmaceutical composition comprising said cyclic compound, or 3) a nucleic acid or vector comprising a nucleic acid encoding the antibody of 1) or the immunogen of 2), to a subject in need thereof.

57.-60. (canceled)

61. An isolated peptide comprising an A beta peptide consisting of the sequence of any one of the sequences set forth in SEQ ID NOS: 1-15, optionally wherein the peptide is a cyclic peptide comprising a linker wherein the linker is covalently coupled to the A-beta peptide N-terminus residue and/or the A-beta C-terminus residue.

62.-65. (canceled)


SEQUENCE LISTING (26 sequences)

SEQ ID NO: 1 - protein, 4 aa, Homo sapiens
GGVV

SEQ ID NO: 2 - protein, 7 aa, Artificial Sequence
  Synthetic Construct
CGGGVVG

SEQ ID NO: 3 - protein, 6 aa, Artificial Sequence
  Synthetic Construct
CGGVVG

SEQ ID NO: 4 - protein, 6 aa, Artificial Sequence
  Synthetic Construct
CGGGVV

SEQ ID NO: 5 - protein, 4 aa, Homo sapiens
VGGV

SEQ ID NO: 6 - protein, 5 aa, Homo sapiens
VGGVV

SEQ ID NO: 7 - protein, 6 aa, Homo sapiens
VGGVVI

SEQ ID NO: 8 - protein, 5 aa, Homo sapiens
GGVVI

SEQ ID NO: 9 - protein, 6 aa, Artificial Sequence
  Synthetic Construct
GGGVVG

SEQ ID NO: 10 - protein, 5 aa, Artificial Sequence
  Synthetic Construct
GGVVG

SEQ ID NO: 11 - protein, 5 aa, Homo sapiens
GGGVV

SEQ ID NO: 12 - protein, 8 aa, Artificial Sequence
  Synthetic Construct
CGGGVVGC

SEQ ID NO: 13 - protein, 11 aa, Homo sapiens
AIIGLMVGGVV

SEQ ID NO: 14 - protein, 6 aa, Homo sapiens
MVGGVV

SEQ ID NO: 15 - protein, 6 aa, Homo sapiens
GGVVIA

SEQ ID NO: 16 - protein, 42 aa, Homo sapiens [sequence omitted]

SEQ ID NO: 17 - protein, 8 aa, Mus musculus
GFTFSNYW

SEQ ID NO: 18 - protein, 10 aa, Mus musculus
IRLKSYNYAT

SEQ ID NO: 19 - protein, 6 aa, Mus musculus
LRWIDY

SEQ ID NO: 20 - protein, 6 aa, Mus musculus
QDINSY

SEQ ID NO: 21 - protein, 3 aa, Mus musculus
RAN

SEQ ID NO: 22 - protein, 9 aa, Mus musculus
PQYDEFPYT

SEQ ID NO: 23 - DNA, 414 bp, Mus musculus
atgtatttgggactgaactgtgtattcatagtttttctcttaaaaggtgtccagagtgaagtgaagcttgaggagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatcttataattatgcaacacattatgcggagtctgtgaaagggaggttcaccatctcaagagatgattccaaaagtagtgtctacctgcaaatgaacaacttaagagctgaagacactggcatttattactgtttacggtggatcgactactggggccaaggcaccactctcacagtctcctcagccaaaacgaca

SEQ ID NO: 24 - protein, 138 aa, Mus musculus
MYLGLNCVFIVFLLKGVQSEVKLEESGGGLVQPGGSMKLSCVASGFTFSNYWMNWVRQSPEKGLEWVAEIRLKSYNYATHYAESVKGRFTISRDDSKSSVYLQMNNLRAEDTGIYYCLRWIDYWGQGTTLTVSSAKTT

SEQ ID NO: 25 - DNA, 399 bp, Mus musculus
atggacatgaggacccctgctcagtttcttggaatcttgttgctctggtttccaggtatcaaatgtgacatcaagatgacccagtctccatcttccatgtatgcatctctaggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtccacagtatgatgagtttccgtacacgttcggaggggggaccaagctggaaataaaacgggctgatgct

SEQ ID NO: 26 - protein, 133 aa, Mus musculus
MDMRTPAQFLGILLLWFPGIKCDIKMTQSPSSMYASLGERVTITCKASQDINSYLSWFQQKPGKSPKTLIYRANRLVDGVPSRFSGSGSGQDYSLTISSLEYEDMGIYYCPQYDEFPYTFGGGTKLEIKRADA